(19) United States
(12) Patent Application Publication
(10) Pub. No.: US 2009/0007281 A1
(43) Pub. Date: Jan. 1, 2009

(54) Animal Model for Assessing Copd-Related Diseases

(75) Inventors: Roger Alan Renne, Sumner, WA (US); Kyeonghee Monica Lee, Richland, WA (US); Katrina Marie Waters, Richland, WA (US); Quanxin Meng, Richland, WA (US); David L. Springer, Pasco, WA (US); Sam Jens Harbo, Kennewick, WA (US); Katherine M. Gideon, Kennewick, WA (US); Joel G. Pounds, Richland, WA (US); Herbert S. Bresler, Columbus, OH (US); Don S. Daly, Richland, WA (US)

Correspondence Address: MACMILLAN, SOBANSKI & TODD, LLC, ONE MARITIME PLAZA-FIFTH FLOOR, 720 WATER STREET, TOLEDO, OH, 43604, US

(73) Assignee: BATTELLE MEMORIAL INSTITUTE, Columbus, OH (US)

(21) Appl. No.: 12/160,672

(22) PCT Filed: Jan. 16, 2007
(86) PCT No.: PCT/US07/01105

§ 371 Date: Jul. 11, 2008

Related U.S. Application Data
(60) Provisional application No. 60/759,250, filed on Jan. 13, 2006.
(60) Provisional application No. 60/779,236, filed on Mar. 3, 2006.
(60) Provisional application No. 60/779,237, filed on Mar. 3, 2006.
(60) Provisional application No. 60/778,658, filed on Mar. 3, 2006.

(51) Int. Cl. A01K 67/027; A01K 67/02; C12Q 1/68; A61K 38/19; C12N 5/06

(52) U.S. Cl. 800/3; 800/9; 435/6; 514/12; 435/325

(57) ABSTRACT

Methods of diagnosis, markers, and screening techniques and animal models for assessing the severity and/or progression or regression of chronic obstructive pulmonary disease (COPD) and COPD-related diseases are disclosed.

### CROSS-REFERENCE TO RELATED APPLICATIONS

This application claims the benefit of U.S. Provisional Application Nos. 60/759,250 filed Jan. 13, 2006; 60/779,236 filed Mar. 3, 2006; 60/779,237 filed Mar. 3, 2006; and, 60/778,658 filed Mar. 3, 2006, the disclosures of which are expressly incorporated herein by reference.

### TECHNICAL FIELD AND INDUSTRIAL APPLICABILITY OF THE INVENTION

Methods of diagnosis, markers and screening techniques and animal models useful therewith are described herein. More particularly, the methods are useful for assessing the severity and/or progression or regression of chronic obstructive pulmonary disease (COPD) and COPD-related diseases. Also, the methods are useful for determining the efficacy of drugs that may be capable of treating said diseases.

### BACKGROUND OF THE INVENTION

Cigarette smoke is the major cause of chronic obstructive pulmonary diseases (COPD) in humans. However, the mechanistic studies of smoke-induced COPD in experimental animals generally fail to provide clear evidence of definitive pathological changes in affected tissue. In addition, these mechanistic studies generally require unduly long-term intensive smoke exposure.

Chronic inflammation is one of the crucial components of COPD-related diseases. Long-term exposure to cigarette smoke is the leading cause of COPD. However, development of COPD in rodents secondary to cigarette smoke exposure takes a long period of exposures and results in mild lung lesions, limiting the usefulness of the animal model for mechanistic research and therapeutic development. In order to heighten the severity of pulmonary lesions and inflammatory responses to mimic COPD progression in human smokers, there is needed a compromised model for use as an animal model for COPD investigation. Lipopolysaccharide (LPS) is a component of the Gram-negative bacterial cell wall and a potent endotoxin capable of activating innate immunity (Martin, 2000).

Because multiple factors may be involved in the development of COPD-related diseases, and because it typically takes at least several months or more for conventional animal models to develop any measurable COPD-related symptoms or complications, the conventional animal models make it difficult and time-consuming to analyze the pathogenesis of COPD-related diseases and thus are often not a suitable as a standard model for COPD-related diseases.

Therefore, despite the fact that great attention has been paid to the development of COPD-related diseases, the causes and potential therapies for COPD-related diseases remains a great concern. Accordingly, it remains an important goal to establish a suitable animal model for COPD-related diseases and to develop new approaches for treating these diseases. There is also a need to investigate the biological effects of acute as well as chronic exposure to cigarette smoke and LPS need to be characterized, especially with regard to the development of COPD-related diseases.

### SUMMARY OF THE INVENTION

In one broad aspect, there is provided herein an animal model that can be used to assess one or more metabolic pathways that contribute to the pathogenesis of COPD and related diseases. In certain embodiments, the animal model is exposed to one or more toxin or chemical sufficient to produce a COPD-related disease response.

There is provided herein markers for detecting the initiation or development of a COPD-related disease (hereinafter “markers” or “biomarkers”), which are listed in various Tables herein. There is also provided herein nucleic acids and proteins that are encoded by or correspond to the markers (hereinafter “marker nucleic acids” and “marker proteins,” respectively).

In another aspect, there is provided herein various methods, reagents and kits for diagnosing, staging, prognosing, monitoring and treating COPD-related diseases.

In one embodiment, there is provided a diagnostic method of assessing whether a patient has a COPD-related disease or has higher than normal risk for developing a COPD-related disease, comprising the steps of comparing the level of expression of a marker in a patient sample and the normal level of expression of the marker in a control, e.g., a sample from a patient without a COPD-related disease. A significantly higher level of expression of the marker in the patient sample as compared to the normal level is an indication that the patient is afflicted with a COPD-related disease or has higher than normal risk for developing a COPD-related disease.

The markers are selected such that the positive predictive value of the methods is at least about 10%, and in certain non-limiting embodiments, about 25%, about 50% or about 90%. Also preferred for use in the methods are markers that are differentially expressed, as compared to normal cells, by at least two-fold in at least about 20%, and in certain non-limiting embodiments, about 50% or about 75%.

In one diagnostic method of assessing whether a patient is afflicted with a COPD-related disease (e.g., new detection (“screening”), detection of recurrence, reflex testing), the method comprises comparing: a) the level of expression of a marker in a patient sample, and b) the normal level of expression of the marker in a control non-COPD-related disease sample. A significantly higher level of expression of the marker in the patient sample as compared to the normal level is an indication that the patient is afflicted with a COPD-related disease.

There is also provided diagnostic methods for assessing the efficacy of a therapy for inhibiting a COPD-related disease in a patient. Such methods comprise comparing: a) expression of a marker in a first sample obtained from the patient prior to providing at least a portion of the therapy to the patient, and b) expression of the marker in a second sample obtained from the patient following provision of the portion of the therapy. A significantly lower level of expression of the marker in the second sample relative to that in the first sample is an indication that the therapy is efficacious for inhibiting a COPD-related disease in the patient.

It will be appreciated that in these methods the “therapy” may be any therapy for treating a COPD-related disease including, but not limited to, pharmaceutical compositions, gene therapy and biologic therapy such as the administering of antibodies and chemokines. Thus, the methods described herein may be used to evaluate a patient before, during and after therapy, for example, to evaluate the reduction in disease state.

In certain aspects, the diagnostic methods are directed to therapy using a chemical or biologic agent. These methods comprise comparing: a) expression of a marker in a first sample obtained from the patient and maintained in the presence of the chemical or biologic agent, and b) expression of the marker in a second sample obtained from the patient and maintained in the absence of the agent. A significantly lower level of expression of the marker in the second sample relative to that in the first sample is an indication that the agent is efficacious for inhibiting a COPD-related disease in the patient. In one embodiment, the first and second samples can be portions of a single sample obtained from the patient or portions of pooled samples obtained from the patient.

There is also provided a monitoring method for assessing the progression of a COPD-related disease in a patient, the method comprising: a) detecting in a patient sample at a first time point, the expression of a marker; b) repeating step a) at a subsequent time point in time; and c) comparing the level of expression detected in steps a) and b), and therefrom monitoring the progression of a COPD-related disease in the patient. A significantly higher level of expression of the marker in the sample at the subsequent time point from that of the sample at the first time point is an indication that the COPD-related disease has progressed, whereas a significantly lower level of expression is an indication that the COPD-related disease has regressed.

There is further provided a diagnostic method for determining whether a COPD-related disease has worsened or is likely to worsen in the future, the method comprising comparing: a) the level of expression of a marker in a patient sample, and b) the normal level of expression of the marker in a control sample. A significantly higher level of expression in the patient sample as compared to the normal level is an indication that the COPD-related disease has worsened or is likely to worsen in the future.

There is also provided a test method for selecting a composition for inhibiting a COPD-related disease in a patient. This method comprises the steps of: a) obtaining a sample comprising cells from the patient; b) separately maintaining aliquots of the sample in the presence of a plurality of test compositions; c) comparing expression of a marker in each of the aliquots; and d) selecting one of the test compositions which significantly reduces the level of expression of the marker in the aliquot containing that test composition, relative to the levels of expression of the marker in the presence of the other test compositions.

There is additionally provided a test method of assessing the harmful potential of a compound in causing a COPD-related disease. This method comprises the steps of: a) maintaining separate aliquots of cells in the presence and absence of the compound; and b) comparing expression of a marker in each of the aliquots. A significantly higher level of expression of the marker in the aliquot maintained in the presence of the compound, relative to that of the aliquot maintained in the absence of the compound, is an indication that the compound possesses such harmful potential.

In addition, there is further provided a method of inhibiting a COPD-related disease in a patient. This method comprises the steps of: a) obtaining a sample comprising cells from the patient; b) separately maintaining aliquots of the sample in the presence of a plurality of compositions; c) comparing expression of a marker in each of the aliquots; and d) administering to the patient at least one of the compositions which significantly lowers the level of expression of the marker in the aliquot containing that composition, relative to the levels of expression of the marker in the presence of the other compositions.

The level of expression of a marker in a sample can be assessed, for example, by detecting the presence in the sample of: the corresponding marker protein or a fragment of the protein (e.g. by using a reagent, such as an antibody, an antibody derivative, an antibody fragment or single-chain antibody, which binds specifically with the protein or protein fragment) the corresponding marker nucleic acid (e.g. a nucleotide transcript, or a complement thereof), or a fragment of the nucleic acid (e.g. by contacting transcribed polynucleotides obtained from the sample with a substrate having affixed thereto one or more nucleic acids having the entire or a segment of the nucleic acid sequence or a complement thereof) a metabolite which is produced directly (i.e., catalyzed) or indirectly by the corresponding marker protein.

Any of the aforementioned methods may be performed using a plurality (e.g. 2, 3, 5, or 10 or more) of COPD-related disease markers, including COPD-related disease markers. In such methods, the level of expression in the sample of each of a plurality of markers, at least one of which is a marker, is compared with the normal level of expression of each of the plurality of markers in samples of the same type obtained from control humans not afflicted with a COPD-related disease. A significantly altered (i.e., increased or decreased as specified in the above-described methods using a single marker) level of expression in the sample of one or more markers, or some combination thereof, relative to that marker's corresponding normal or control level, is an indication that the patient is afflicted with a COPD-related disease. For all of the aforementioned methods, the marker(s) are selected such that the positive predictive value of the method is at least about 10%.

In another aspect, there is provided various diagnostic and test kits. In one embodiment, a kit is useful for assessing whether a patient is afflicted with a COPD-related disease. The kit comprises a reagent for assessing expression of a marker. In another embodiment, a kit is useful for assessing the suitability of a chemical or biologic agent for inhibiting a COPD-related disease in a patient. Such a kit comprises a reagent for assessing expression of a marker, and may also comprise one or more of such agents. In a further embodiment, the kits are useful for assessing the presence of COPD-related disease cells or treating COPD-related diseases. Such kits comprise an antibody, an antibody derivative or an antibody fragment, which binds specifically with a marker protein or a fragment of the protein. Such kits may also comprise a plurality of antibodies, antibody derivatives or antibody fragments wherein the plurality of such antibody agents binds specifically with a marker protein or a fragment of the protein.

In an additional embodiment, the kits are useful for assessing the presence of COPD-related disease cells, wherein the kit comprises a nucleic acid probe that binds specifically with a marker nucleic acid or a fragment of the nucleic acid. The kit may also comprise a plurality of probes, wherein each of the probes binds specifically with a marker nucleic acid, or a fragment of the nucleic acid.

In a further aspect, there is provided methods for treating a patient afflicted with a COPD-related disease or at risk of developing a COPD-related disease. Such methods may comprise reducing the expression and/or interfering with the biological function of a marker. In one embodiment, the method comprises providing to the patient an antisense oligonucleotide or polynucleotide complementary to a marker nucleic acid, or a segment thereof. For example, an antisense polynucleotide may be provided to the patient through the delivery of a vector that expresses an anti-sense polynucleotide of a marker nucleic acid or a fragment thereof. In another embodiment, the method comprises providing to the patient an antibody, an antibody derivative or antibody fragment, which binds specifically with a marker protein, or a fragment of the protein.

In a broad aspect, there is provided a method for producing a non-human animal model for assessment of at least one COPD-related disease. The method includes exposing the animal to repeated doses of at least one chemical found in smoke and at least one toxin. In certain aspect, the method further includes collecting one or more selected samples from the animal; and comparing the collected sample to one or more indicia of potential COPD initiation or development.

In broad aspect, there is provides a method of producing the animal model that includes: maintaining the animal in a specific chemical-free environment and sensitizing the animal with at least one chemical found in smoke and at least one toxin. In certain embodiments, at least a part of the animal's respiratory system is sensitized by multiple sequential exposures.

In another broad aspect, there is provided a method of screening for an agent for effectiveness against at least one COPD-related disease. The method generally includes: administering at least one agent to the animal, determining whether the agent reduces or aggravates one or more symptoms of the COPD-related disease; correlating a reduction in one or more symptoms with effectiveness of the agent against the COPD-related disease; or correlating a lack of reduction in one or more symptoms with ineffectiveness of the agent.

Also provided is a method of providing a non-human animal model for at least one COPD-related disease complication where the method comprising exposing to the non-human animal at least one chemical found in smoke and lipopolysaccharide (LPS) in an amount sufficient to induce the at least one complication in the animal. In certain embodiments, the complication manifests in the animal at least about a month earlier than that in an available animal model not exposed to the at least one chemical and LPS. In other embodiments, the COPD-related disease complication manifests in the animal at least about 3 weeks after exposure.

In certain embodiments, the animal's lungs are infiltrated with at least one of the LPS toxin and at least one of the smoke chemicals such that an inflammatory response occurs. The symptoms include expressing, in a measurable amount, at least one or more measurable markers, or a functional equivalent thereto, that has been increased or decreased in the animal model.

The method can include: comparing pathology changes in the animal, and identifying a level of infiltration of the chemical and toxin in respiratory system tissue of the animal with one or more of increased inflammatory response, increased macrophage activity, and altered level of neutrophil infiltration. The method can also include alternating exposures of the chemical and the toxin in a manner sufficient to initiate a COPD-related disease response in the animal model.

Also, the method can further include: a) repeating doses of the smoke chemical for at least one period during a number of successive days, followed by repeating doses of the LPS toxin for at least one period during successive days; and, optionally, b) repeating the previous step at least two additional times. The step a) can include controlling a delivered amount of the LPS toxin by producing inhalable quantities of the LPS toxin and, optionally, combining the inhalable LPS toxin with heated diluted air.

In certain methods, in response to data generated, the method can further include at least a further step of adjusting the amount and/or time exposure of the animal. Also, these steps can be performed simultaneously.

The chemicals can be found in one or more of: tobacco, a non-tobacco smoking product, an actively oxidizing material producing an inhalable particle, an inhalable chemical, a vaporized material, a droplet of material, and an inhalable particle. In certain embodiments, the chemical comprises one or more chemicals found in cigarette tobacco smoke.

In certain embodiments, the animal's lungs are infiltrated with at least one of the toxin and at least one of the smoke chemicals, whereby an inflammatory response occurs. The animal model can be a nonhuman vertebrate, including a mammal such as a mouse, rat, rabbit, or primate. The exposure causes at least a differential inflammatory response in the animal without causing acute morbidity.

The animal model is useful for assessing one or more metabolic pathways that contribute to at least one of initiation, progression, severity, pathology, aggressiveness, grade, activity, disability, mortality, morbidity, disease sub-classification or other underlying pathogenic or pathological feature of at least one COPD-related disease. One or more of the following biological or chemical processes occurs in the animal model after exposure of the animal to at least one chemical found in smoke and at least one toxin: i) decreased heat shock response and/or chaperone activity, ii) altered immune and inflammatory response, iii) increased cell proliferation; iv) unchecked immune regulation of inflammatory response, v) calcium homeostasis imbalance, vi) cell death versus proliferation imbalance affecting several cell types, vii) protease activity, viii) decreased macrophage function, and ix) imbalance of oxidant to antioxidant potential.

The analysis can be by one or more of: hierarchical clustering, signature network construction, mass spectroscopy proteomic analysis, surface plasmon resonance, linear statistical modeling, partial least squares discriminant analysis, and multiple linear regression analysis.

In certain embodiments, the method includes one or more of: blood as the sample analyzed for one or more of carboxyhemoglobin (COHb), nicotine and cotinine; lung tissue as the sample analyzed for at least one of bronchoalveolar lavage (BAL), histopathology or immunohistology; and BAL fluid (BALF) as the sample analyzed for at least one of enzymes, total protein, cytology and cytokines.

In a particular aspect, the animal model is assessed for at least one COPD-related disease, by examining an expression level of one or more markers, or a functional equivalent thereto. The marker has been increased or decreased in the animal model after exposure of the animal to the at least one chemical found in smoke and the at least one toxin including LPS. The marker comprises one or more of: i) one or more of the BAL cytokines, or fragments or functional equivalents thereof, as shown in TABLE 8; ii) one or more genes, or fragments or functional equivalents thereof as shown in at least one of TABLES 14, 15 and 16; iii) one or more genes, or fragments or functional equivalents thereof, encoding for a protein, or fragment or antibody that binds to, as shown in at least one of TABLES 19, 20 and 21; and, iv) one or more genes, or fragments or functional equivalents thereof, encoding for a protein, or fragment or antibody that binds thereto, that produces one or more of the biological or chemical processes as shown in at least one of TABLES 17, 18, 22 and 23.

In certain embodiments, the at least one marker is differentially expressed at a 2-fold change or greater, and in certain embodiments; in particular embodiments at least one marker is differentially expressed at a 10-fold change or greater.

In another aspect, there is provides a method of assessing the potential of at least one chemical found in smoke for an ability to initiate a COPD-related disease response in an animal model. The assessment method can include: 1) measuring one or more of up- or down-regulated markers (or fragments thereof) after exposure of the animal to one or more of: i) at least one smoke chemical, and ii) lipopolysaccharide (LPS) in amounts sufficient to initiate a COPD-related disease response in the animal; and, 2) determining whether the up- or down-regulated markers has the ability to initiate a COPD-related disease response.

In another aspect, there is provided a method of screening for a therapeutic agent for treatment of at least one COPD-related disease that includes: 1) assaying for an expression level of a marker in a sample obtained from the animal, and 2) comparing the expression levels assayed to that in a control with which the candidate therapeutic agent has not been contacted. The sample can be one or more of blood, plasma, serum, urine, saliva, bronchoalveolar lavage (BAL), exhaled breath, or exhaled breath condensate. Also, the sample comprises lung tissue or other tissue of the respiratory system.

The assaying step can include: a) determining an initial level of one or more markers in a first sample from the animal, b) determining a subsequent level of the one or more markers in a second sample from the animal after administration of the candidate therapeutic agent; and c) determining whether the subsequent level of the one or more markers in the sample is higher or lower than the initial level of the marker in the first sample.

In certain embodiments, the second sample is obtained at least about six hours after administering the candidate therapeutic agent. In other embodiments, the second sample is obtained no more than about six months after administering the candidate therapeutic agent.

In another aspect, there is provided a method of assessing the effectiveness of a therapy to prevent, diagnose and/or treat at least one COPD-related disease. Such method can include: 1) subjecting the animal model to a regimen whose effectiveness is being assessed, and 2) determining the level of effectiveness of the treatment being tested in treating or preventing the COPD-related disease. The therapy being assessed is useful for human subjects. In certain embodiments, the method is neither a method for the treatment of the human or animal body by surgery or therapy nor a diagnostic method practiced on the human or animal body. The candidate therapeutic agent can be one or more of: pharmaceutical compositions, nutraceutical compositions, and homeopathic compositions.

In yet another aspect, there is provided a method of screening for a therapeutic agent useful for treating or preventing a COPD-related disease complication. The method can include, providing a test animal and a substantially identical control animal; maintaining the test animal and the control animal under conditions appropriate for development of at least one COPD-related disease complication in the control animal; assessing the at least one COPD-related disease complication in the test animal and the control animal; and, comparing the severity and/or onset of the COPD-related disease complication in the test animal with that of the control animal. A reduced severity and/or delay in the onset of the COPD-related disease complication in the test animal indicates that the candidate agent is the therapeutic agent useful for treating or preventing the COPD-related disease complication.

Also, there is provided a method of screening for an agent for effectiveness against at least one COPD-related disease which can include: i) administering at least one agent to the animal, ii) determining whether the agent reduces or aggravates one or more symptoms of the COPD-related disease; iii) correlating a reduction in one or more symptoms with effectiveness of the agent against the COPD-related disease; or iv) correlating a lack of reduction in one or more symptoms with ineffectiveness of the agent. In certain embodiments, macrophage infiltration is determined.

### DESCRIPTION OF THE FIGURES

FIG. 1 is a graph comparing mean body weight versus study day for sham control, LPS, Smoke and Smoke+LPS.

FIG. 2 is a photograph that shows mixed inflammatory infiltrate.

FIG. 3 is a photograph that shows diffuse mixed inflammatory cell infiltrate.

FIG. 4 is a photograph that shows the diffuse cellular infiltrate composed of predominately neutrophils in lung sections for the LPS group.

FIG. 5-1 is a photograph that shows Phase 1-Animal 0006-control-20X.

FIG. 5-2 is a photograph that shows Phase 1-Animal 0006-LPS-20X.

FIG. 5-3 is a photograph that shows Phase 2-Animal 0009-control-20X.

FIG. 5-4 is a photograph that is a photograph that shows Phase 2-Animal 0009-LPS-20X.

FIG. 5-5 is a photograph that shows Phase 2-Animal 0409-Smoke-20X.

FIG. 5-6 is a photograph that shows Phase 2-Animal 0611-Smoke+LPS-20X.

FIG. 5-7 is a photograph that show s Phase 2-Animal 0610-Smoke+LPS-20X.

FIG. 8 shows a Venn diagram of overlapping microarray data.

FIG. 7 is a graph that shows a statistical analysis of genes to distinguish the groups: Smoke: Cxcl5 [SEQ ID No. 121], Zranb3 [SEQ ID No. 122], Eraf [SEQ ID No. 123]; LPS: Cxcl9 [SEQ ID No. 127], Saa1 [SEQ ID No. 1], Cxcl11 [SEQ ID No. 128]; and Smoke+LPS: Fshprh1 [SEQ ID No. 124], Ccnb-rs1 [SEQ ID No. 125], Tnfrsf10b [SEQ ID No. 126].

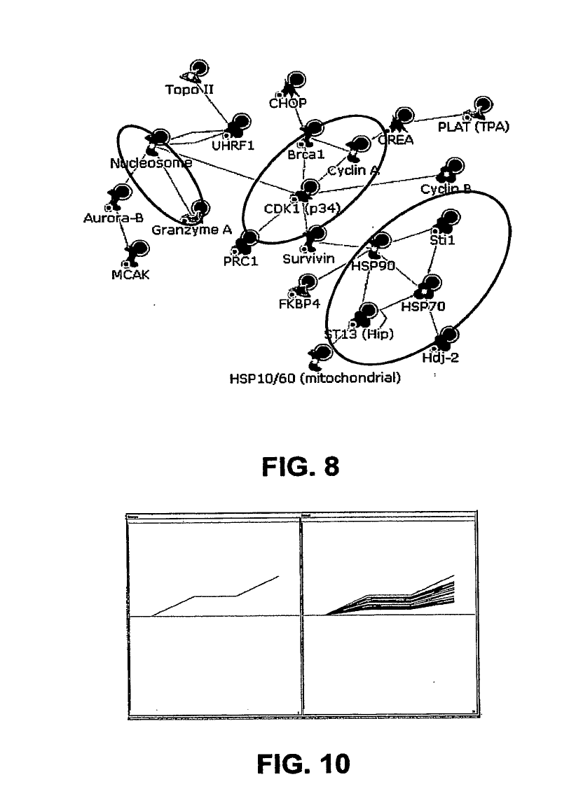

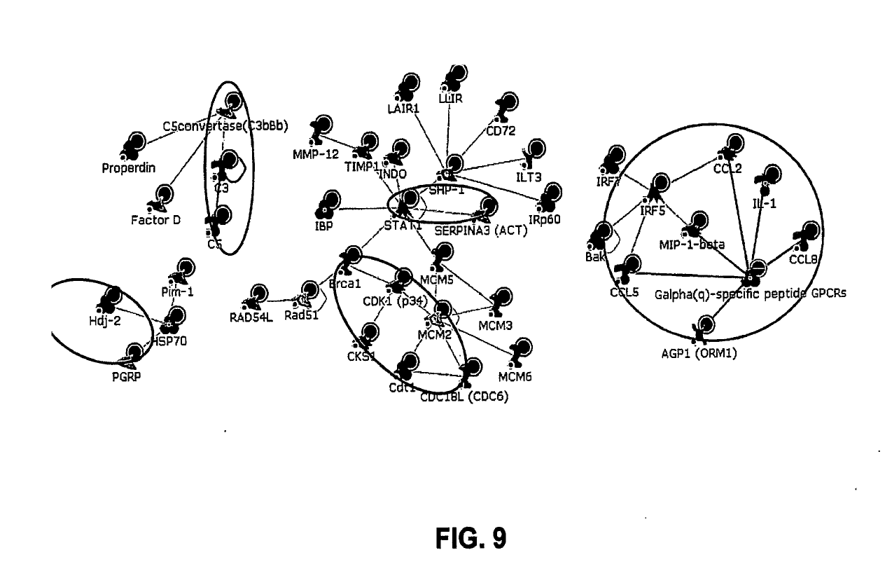

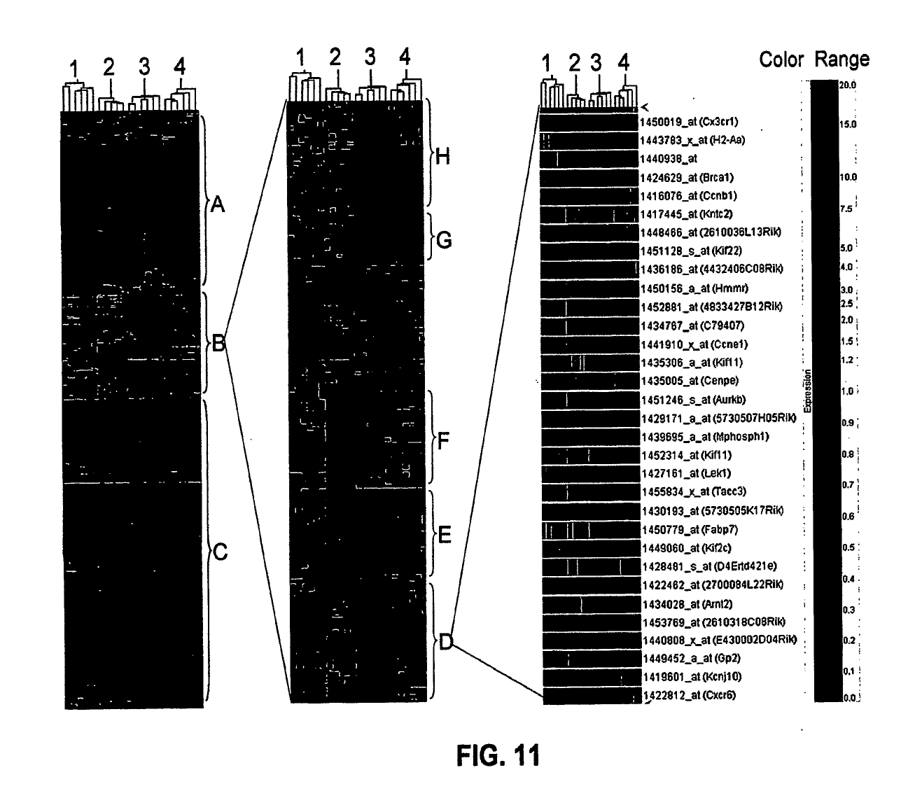

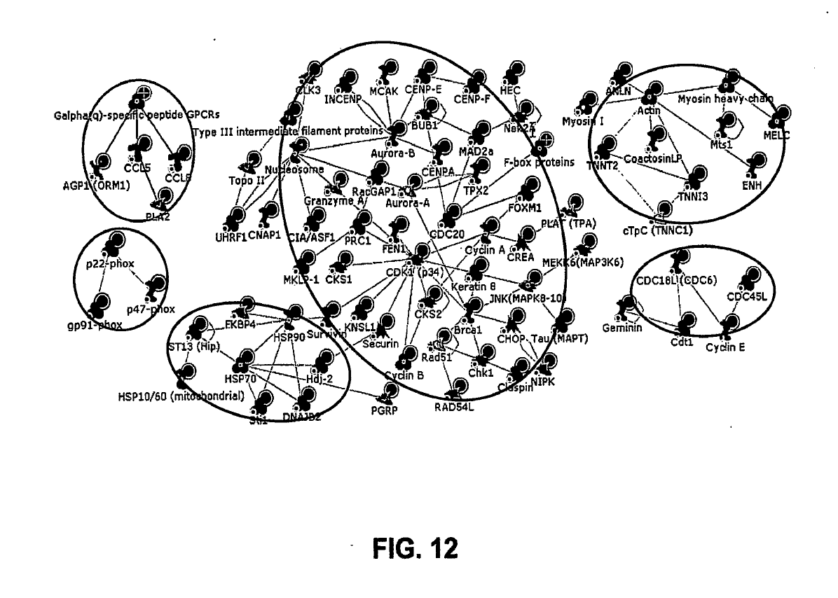

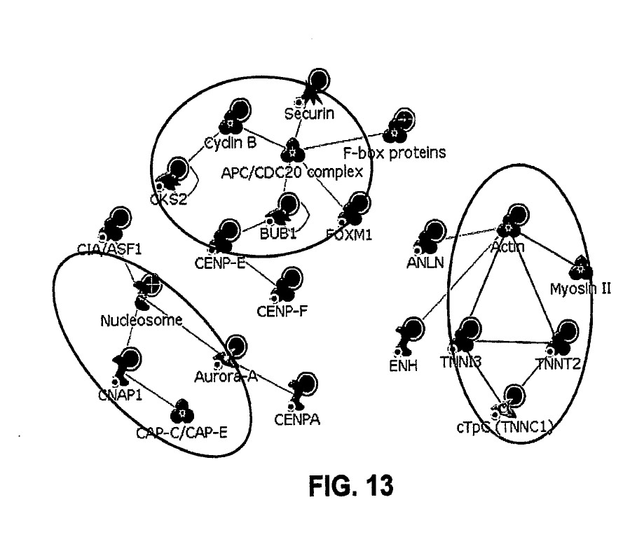

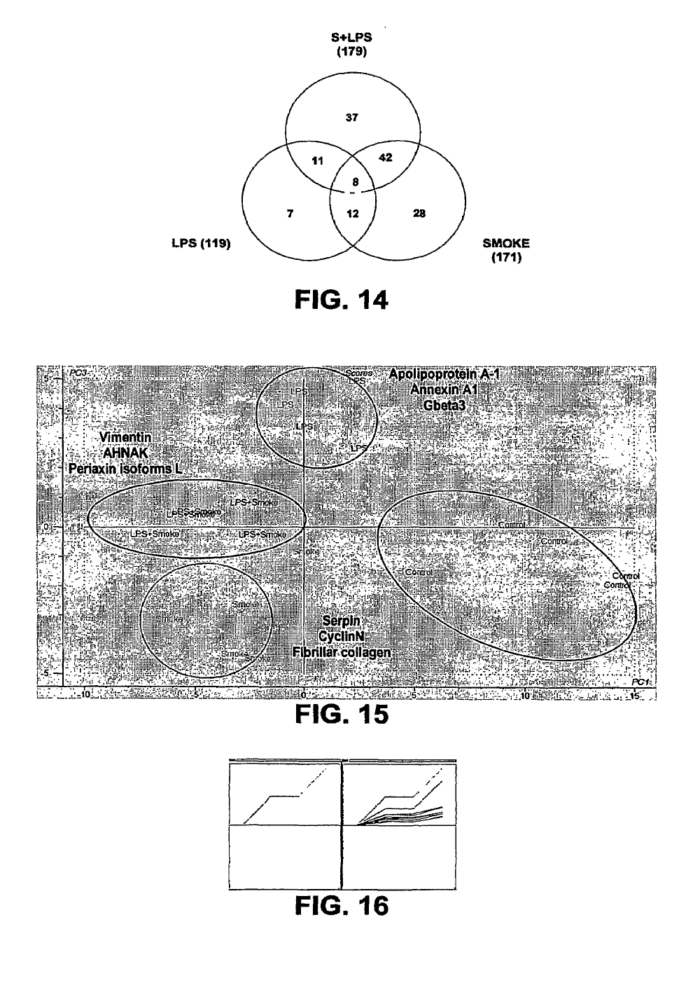

FIG. 8 shows the gene networks after applying the direct interactions algorithm to the list of differentially expressed genes in the Smoke group. One single network was generated. Colored symbols represent genes (network nodes). Red solid circles represent up-regulated genes and blue solid circles represent down-regulated genes. Primary function modules are highlighted with green circles. Primary function modules include heat shock response (HSP70, HSP90, etc), mitotic process (CDK1 and Cyclin A, etc.), DNA damage check point Nucloesome, Granzyme A, etc.).

FIG. 9 shows the gene networks after applying the direct interactions algorithm to the list of differentially expressed genes in the LPS group. Four networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

FIG. 10 is a graph which shows the Pearson correlation distance metrics used for similarity search; tolerance level: 2% (25 profiles).

FIG. 11 shows the hierarchical clustering of genes and treatment conditions. The sums of differentially expressed genes in three treatment groups (940 genes) were used for the analysis. 1, sham control group; 2, LPS group; 3, Smoke+LPS group; 4, Smoke group. Clusters A, B, and C are the first level branches in the gene tree, and clusters D to H are sub-clusters within cluster B. Color range is based on raw signal intensity value of a gene divided by a normalization factor; red represent genes expressed at high level and green represent genes expressed at low level.

FIG. 12 shows the gene networks after applying the direct interactions algorithm to the list of differentially expressed genes in the Smoke+LPS group. One large network and four smaller networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

FIG. 13 shows the subtraction of the Smoke network and the LPS network from the Smoke+LPS network by logical operation. Three small networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

FIG. 14 shows a Venn diagram of overlapping total proteins identified data.

FIG. 15 is a graph that shows a statistical analysis of peptides to distinguish the groups: Smoke: Serpin, CyclinN, Fibrillar collagen; LPS: Apolipoprotein A-1, Annexin A-1, Gbeta3; and Smoke+LPS: Vimentin; AJNAK, Periaxin isoforms L.

FIG. 16 is a graph which shows the Pearson correlation distance metrics used for similarity search; tolerance level: 5% (11 profiles).

### DESCRIPTION OF THE PREFERRED EMBODIMENTS

Unless otherwise defined, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (Glover ed., 1985); Oligonucleotide Synthesis (Gait ed., 1984); Mullis et al. U.S. Pat. No. 4,683,195; Nucleic Acid Hybridization (Hames & Higgins eds., 1984); Transcription And Translation (Hames & Higgins eds., 1984); Culture Of Animal Cells (R. I. Freshney, Alan R. Liss, Inc., 1987); Immobilized Cells And Enzymes (IRL Press, 1986); Perbal, A Practical Guide To Molecular Cloning (1984); the treatise, Methods In Enzymology (Academic Press, Inc., N.Y.); Gene Transfer Vectors For Mammalian Cells (Miller and Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (Weir and Blackwell, eds., 1986); The Laboratory Rat, editor in chief: Mark A. Suckow; authors: Sharp and LaRegina. CRC Press, Boston. 1988, which are incorporated herein by reference) and chemical methods.

Described herein are newly discovered markers associated with a COPD-induced state of various cells. It has been discovered that the higher than normal level of expression of any of these markers or combination of these markers correlates with the presence of a COPD-related disease in a patient. Methods are provided for detecting the presence of a COPD-related disease in a sample; the absence of a COPD-related disease in a sample; the stage of a COPD-related disease; and, other characteristics of a COPD-related disease that are relevant to the assessment, prevention, diagnosis, characterization and therapy of a COPD-related disease in a patient. Methods of treating a COPD-related disease are also provided.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

A “marker” is a gene or protein whose altered level of expression in a tissue or cell from its expression level in normal or healthy tissue or cell is associated with a disease state.

The “normal” level of expression of a marker is the level of expression of the marker in respiratory system cells of a human subject or patient not afflicted with a COPD-related disease.

An “over-expression” or “significantly higher level of expression” of a marker refers to an expression level in a test sample that is greater than the standard error of the assay employed to assess expression, and in certain embodiments, at least twice, and in other embodiments, three, four, five or ten times the expression level of the marker in a control sample (e.g., sample from a healthy subject not having the marker associated disease) and in certain embodiments, the average expression level of the marker in several control samples.

A “significantly lower level of expression” of a marker refers to an expression level in a test sample that is at least twice, and in certain embodiments, three, four, five or ten times lower than the expression level of the marker in a control sample (e.g., sample from a healthy subject not having the marker associated disease) and in certain embodiments, the average expression level of the marker in several control samples.

A kit is any manufacture (e.g. a package or container) comprising at least one reagent, e.g. a probe, for specifically detecting the expression of a marker. The kit may be promoted, distributed or sold as a unit for performing the methods of the present invention.

“Proteins” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The compositions, kits and methods described herein have the following uses, among others: 1) assessing whether a patient is afflicted with a COPD-related disease; 2) assessing the stage of a COPD-related disease in a human patient; 3) assessing the grade of a COPD-related disease in a patient; 4) assessing the nature of a COPD-related disease in a patient; 5) assessing the potential to develop a COPD-related disease in a patient; 6) assessing the histological type of cells associated with a COPD-related disease in a patient; 7) making antibodies, antibody fragments or antibody derivatives that are useful for treating a COPD-related disease and/or assessing whether a patient is afflicted with a COPD-related disease; 8) assessing the presence of COPD-related disease cells; 9) assessing the efficacy of one or more test compounds for inhibiting a COPD-related disease in a patient; 10) assessing the efficacy of a therapy for inhibiting a COPD-related disease in a patient; 11) monitoring the progression of a COPD-related disease in a patient; 12) selecting a composition or therapy for inhibiting a COPD-related disease in a patient; 13) treating a patient afflicted with a COPD-related disease; 14) inhibiting a COPD-related disease in a patient; 15) assessing the harmful potential of a test compound; and 16) preventing the onset of a COPD-related disease in a patient at risk for developing a COPD-related disease.

### OVERVIEW

Methods for Generating Non-Human Animal Models for Inducing at least One Indication (or Multiple Indicia) of a COPD-Related Disease Response.

The Global Initiative for Chronic Obstructive Lung Disease (GOLD) proposes a definition of COPD that focuses on the progressive nature of airflow limitation and its association with abnormal inflammatory response of the lungs to various noxious particles or gases. According to the GOLD document, COPD is defined as “a disease state characterized by airflow limitation that is not fully reversible. The airflow limitation is usually both progressive and associated with an abnormal inflammatory response of the lungs to noxious particles or gases.” American Thoracic Society. Standards for the diagnosis and care of patients with chronic obstructive pulmonary disease. Am J Respir Crit. Care Med. 1995; 152:S77-S121. Siafakas N M, Vermeire P, Pride N B, et al., for the European Respiratory Society Task Force. Optimal assessment and management of chronic obstructive pulmonary disease (COPD). Eur Respir J. 1995; 8:1398-1420. The COPD Guidelines Group of the Standards of Care Committee of the BTS. BTS guidelines for the management of chronic obstructive pulmonary disease. Thorax. 1997; 52 (suppl 5):S1-S28. Pauwels R A, Buist A S, Calverley P M, Jenkins C R, Hurd S S; GOLD Scientific Committee. Global strategy for the diagnosis, management, and prevention of chronic obstructive pulmonary disease: NHLBI/WHO Global Initiative for Chronic Obstructive Lung Disease (GOLD) Workshop summary. Am J Respir Crit. Care Med. 2001; 163:1256-1276.

There is provided herein methods for producing non-human animal models for inducing at least one indication (or multiple indicia) of a COPD-related disease response. Such non-human animal models exhibit at least one (one or more) indicia of such disease response. In the methods, a non-human animal is exposed to at least one chemical (such as cigarette smoke) and lipopolysaccharide in an amount sufficient to induce a COPD-related disease response in the animal.

The non-human animal can be a rodent, such as a rat or a mouse. A non-human animal may also be another mammal, including, for example, a hamster, a guinea pig, a horse, a pig, a goat, a sheep or other non-human primates. For ease of description, a mouse will be used as the model animal throughout the application to illustrate the invention.

In certain embodiments, the methods animal models have a (at least one, one or more) COPD-related disease response which occurs earlier and/or with greater severity than in currently available mouse models, such as those described herein. In certain further embodiments, methods of the invention produce rat models in which the average time to develop a COPD-related disease response is 6 months, 5 months, 4 months, 3 months, 2 months, 1 month or less than the average time in which a currently available animal model develops corresponding or equivalent COPD-related disease responses. In certain further embodiments, methods of the invention produce animal models in which the average time to develop a COPD-related disease response is about 6 weeks, 5 weeks, 4 weeks, 3 weeks or less.

In certain embodiments, the methods generate animal models that develop at least one COPD-related disease response in the absence of acute morbidity or mortality.

Screening Methods

The animal models created by the methods described herein will enable screening of therapeutic agents useful for treating or preventing a COPD-related disease. Accordingly, the methods are useful for identifying therapeutic agents for treating or preventing a COPD-related disease. The methods comprise administering a candidate agent to an animal model made by the methods described herein, assessing at least one COPD-related disease response in the animal model as compared to a control animal model to which the candidate agent has not been administered. If at least one COPD-related disease response is reduced in symptoms or delayed in onset, the candidate agent is an agent for treating or preventing the COPD-related disease.

The candidate agents may be pharmacologic agents already known in the art or may be agents previously unknown to have any pharmacological activity. The agents may be naturally arising or designed in the laboratory. They may be isolated from microorganisms, animals or plants, or may be produced recombinantly, or synthesized by any suitable chemical method. They may be small molecules, nucleic acids, proteins, peptides or peptidomimetics. In certain embodiments, candidate agents are small organic compounds having a molecular weight of more than 50 and less than about 2,500 daltons. Candidate agents comprise functional groups necessary for structural interaction with proteins. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. There are, for example, numerous means available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. In certain embodiments, the candidate agents can be obtained using any of the numerous approaches in combinatorial library methods art, including, by non-limiting example: biological libraries; spatially addressable parallel solid phase or solution phase libraries; synthetic library methods requiring deconvolution; the “one-bead one-compound” library method; and synthetic library methods using affinity chromatography selection.

In certain further embodiments, certain pharmacological agents may be subjected to directed or random chemical modifications, such as acylation, alkylation, esterification, amidification, etc. to produce structural analogs.

The same methods for identifying therapeutic agents for treating a COPD-related disease can also be used to validate lead compounds/agents generated from in vitro studies.

The candidate agent may be an agent that up- or down-regulates one or more COPD-related disease response pathways. In certain embodiments, the candidate agent may be an antagonist that affects such pathway.

Methods for Treating a COPD-Related Disease

There is provided herein methods for treating, inhibiting, relieving or reversing a COPD-related disease response. In the methods described herein, an agent that interferes with a signaling cascade is administered to an individual in need thereof, such as, but not limited to, COPD-related disease patients in whom such complications are not yet evident and those who already have at least one COPD-related disease response.

In the former instance, such treatment is useful to prevent the occurrence of such COPD-related disease response and/or reduce the extent to which they occur. In the latter instance, such treatment is useful to reduce the extent to which such COPD-related disease response occurs, prevent their further development or reverse the COPD-related disease response.

In certain embodiments, the agent that interferes with the COPD-related disease response cascade may be an antibody specific for such response.

Methods for Validating Therapeutic Agents of COPD-Related Disease Response Using AKR/J Mice

As shown in more detail in the Exemplification section, Applicants have demonstrated that the AKR/J mice animal models made according to the methods described herein show classic COPD-related disease response progression, including complications that are similar to those seen in human patients. Accordingly, there is provided herein further methods for validating lead compounds/agents generated from in vitro studies.

The animal can be a mouse such as an AKR/J strain mouse. In certain methods, the test animal and the control animal are littermates. Also, the animal model develops the at least one COPD-related disease complication in the absence of severe acute morbidity.

In the methods, the animal model as described herein is administered a lead compound for treating a COPD-related disease response at various stages of its life, and maintained under conditions appropriate for such mice. At appropriate time points, the animal model is examined for one or more COPD-related disease responses. If the animal model shows reduced symptoms compared to a control animal that did not receive the lead compound, the lead compound is a validated compound for treating a COPD-related disease response.

All patents, patent applications and references cited herein are incorporated in their entirety by reference. While the invention has been described and exemplified in sufficient detail for those skilled in this art to make and use it, various alternatives, modifications and improvements should be apparent without departing from the spirit and scope of the invention. One skilled in the art readily appreciates that the present invention is well adapted to carry out the objects and obtain the ends and advantages mentioned, as well as those inherent therein. The methods and reagents described herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims. It will also be readily apparent to a person skilled in the art that varying substitutions and modifications may be made to the invention disclosed herein without departing from the scope and spirit of the invention.

It should be understood that although the present invention has been specifically disclosed by preferred embodiments and optional features, modifications and variations of the concepts herein disclosed may be resorted to by those skilled in the art, and that such modifications and variations are considered to be within the scope of this invention as defined by the appended claims.

### EXEMPLIFICATION

The invention now being generally described, it will be more readily understood by reference to the following examples, which are included merely for purposes of illustration of certain embodiments and embodiments of the present invention, and are not intended to limit the invention.

Introduction

Expression of a marker can be inhibited in a number of ways, including, by way of a non-limiting example, an antisense oligonucleotide can be provided to the COPD-related disease cells in order to inhibit transcription, translation, or both, of the marker(s). Alternately, a polynucleotide encoding an antibody, an antibody derivative, or an antibody fragment which specifically binds a marker protein, and operably linked with an appropriate promoter/regulator region, can be provided to the cell in order to generate intracellular antibodies which will inhibit the function or activity of the protein. The expression and/or function of a marker may also be inhibited by treating the COPD-related disease cell with an antibody, antibody derivative or antibody fragment that specifically binds a marker protein. Using the methods described herein, a variety of molecules, particularly including molecules sufficiently small that they are able to cross the cell membrane, can be screened in order to identify molecules which inhibit expression of a marker or inhibit the function of a marker protein. The compound so identified can be provided to the patient in order to inhibit COPD-related disease cells of the patient.

Any marker or combination of markers, as well as any certain markers in combination with the markers, may be used in the compositions, kits and methods described herein. In general, it is desirable to use markers for which the difference between the level of expression of the marker in COPD-related disease cells and the level of expression of the same marker in normal respiratory system cells is as great as possible. Although this difference can be as small as the limit of detection of the method for assessing expression of the marker, it is desirable that the difference be at least greater than the standard error of the assessment method, and, in certain embodiments, a difference of at least 2-, 3-, 4-, 5-, 6-, 7-, 8-, 9-, 10-, 15-, 20-, 100-, 500-, 1000-fold or greater than the level of expression of the same marker in normal tissue.

It is recognized that certain marker proteins are secreted to the extracellular space surrounding the cells. These markers are used in certain embodiments of the compositions, kits and methods, owing to the fact that such marker proteins can be detected in a COPD-associated body fluid sample, which may be more easily collected from a human patient than a tissue biopsy sample. In addition, in vivo techniques for detection of a marker protein include introducing into a subject a labeled antibody directed against the protein. For example, the antibody can be labeled with a radioactive marker whose presence and location in a subject can be detected by standard imaging techniques.

In order to determine whether any particular marker protein is a secreted protein, the marker protein is expressed in, for example, a mammalian cell, such as a human respiratory system line, extracellular fluid is collected, and the presence or absence of the protein in the extracellular fluid is assessed (e.g. using a labeled antibody which binds specifically with the protein).

It will be appreciated that patient samples containing respiratory system cells may be used in the methods described herein. In these embodiments, the level of expression of the marker can be assessed by assessing the amount (e.g. absolute amount or concentration) of the marker in a sample. The cell sample can, of course, be subjected to a variety of post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

It will also be appreciated that the markers may be shed from the cells into the blood stream and/or interstitial spaces. The shed markers can be tested, for example, by examining the serum or plasma.

The compositions, kits and methods can be used to detect expression of marker proteins having at least one portion which is displayed on the surface of cells which express it. For example, immunological methods may be used to detect such proteins on whole cells, or computer-based sequence analysis methods may be used to predict the presence of at least one extracellular domain (i.e. including both secreted proteins and proteins having at least one cell-surface domain). Expression of a marker protein having at least one portion which is displayed on the surface of a cell which expresses it may be detected without necessarily lysing the cell (e.g. using a labeled antibody which binds specifically with a cell-surface domain of the protein).

Expression of a marker may be assessed by any of a wide variety of methods for detecting expression of a transcribed nucleic acid or protein. Non-limiting examples of such methods include immunological methods for detection of secreted, cell-surface, cytoplasmic or nuclear proteins, protein purification methods, protein function or activity assays, nucleic acid hybridization methods, nucleic acid reverse transcription methods and nucleic acid amplification methods.

In a particular embodiment, expression of a marker is assessed using an antibody (e.g. a radio-labeled, chromophore-labeled, fluorophore-labeled or enzyme-labeled antibody), an antibody derivative (e.g. an antibody conjugated with a substrate or with the protein or ligand of a protein-ligand pair), or an antibody fragment (e.g. a single-chain antibody, an isolated antibody hypervariable domain, etc.) which binds specifically with a marker protein or fragment thereof, including a marker protein which has undergone all or a portion of its normal post-translational modification.

In another particular embodiment, expression of a marker is assessed by preparing mRNA/cDNA (i.e. a transcribed polynucleotide) from cells in a patient sample, and by hybridizing the mRNA/cDNA with a reference polynucleotide which is a complement of a marker nucleic acid, or a fragment thereof cDNA can, optionally, be amplified using any of a variety of polymerase chain reaction methods prior to hybridization with the reference polynucleotide; preferably, it is not amplified. Expression of one or more markers can likewise be detected using quantitative PCR to assess the level of expression of the marker(s). Alternatively, any of the many methods of detecting mutations or variants (e.g. single nucleotide polymorphisms, deletions, etc.) of a marker may be used to detect occurrence of a marker in a patient.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker nucleic acid. If polynucleotides complementary to or homologous with are differentially detectable on the substrate (e.g. detectable using different chromophores or fluorophores, or fixed to different selected positions), then the levels of expression of a plurality of markers can be assessed simultaneously using a single substrate (e.g. a “gene chip” microarray of polynucleotides fixed at selected positions). When a method of assessing marker expression is used which involves hybridization of one nucleic acid with another, it is desired that the hybridization be performed under stringent hybridization conditions.

In certain embodiments, the biomarker assays can be performed using mass spectrometry or surface plasmon resonance. In various embodiment, the method of identifying an agent active against a COPD-related disease can include a) providing a sample of cells containing one or more markers or derivative thereof; b) preparing an extract from said cells; c) mixing said extract with a labeled nucleic acid probe containing a marker binding site; and, d) determining the formation of a complex between the marker and the nucleic acid probe in the presence or absence of the test agent. The determining step can include subjecting said extract/nucleic acid probe mixture to an electrophoretic mobility shift assay. In certain embodiments, the determining step comprises an assay selected from an enzyme linked immunoabsorption assay (ELISA), fluorescence based assays and ultra high throughput assays, for example surface plasmon resonance (SPR) or fluorescence correlation spectroscopy (FCS) assays. In such embodiments the SPR sensor is useful for direct real-time observation of biomolecular interactions since SPR is sensitive to minute refractive index changes at a metal-dielectric surface. SPR is a surface technique that is sensitive to changes of 10⁵ to 10⁻⁶ refractive index (RI) units within approximately 200 nm of the SPR sensor/sample interface. Thus, SPR spectroscopy is useful for monitoring the growth of thin organic films deposited on the sensing layer.

Because the compositions, kits, and methods rely on detection of a difference in expression levels of one or more markers, it is desired that the level of expression of the marker is significantly greater than the minimum detection limit of the method used to assess expression in at least one of normal cells and COPD-affected cells.

It is understood that by routine screening of additional patient samples using one or more of the markers, it will be realized that certain of the markers are over-expressed in cells of various types, including specific COPD-related diseases. For example, it will be confirmed that some of the markers are over-expressed in most (i.e. 50% or more) or substantially all (i.e. 80% or more) of a COPD-related disease.

In addition, as a greater number of patient samples are assessed for expression of the markers and the outcomes of the individual patients from whom the samples were obtained are correlated, it will also be confirmed that altered expression of certain of the markers are strongly correlated with a COPD-related disease and that altered expression of other markers are strongly correlated with other diseases. The compositions, kits, and methods are thus useful for characterizing one or more of the stage, grade, histological type, and nature of a COPD-related disease in patients.

When the compositions, kits, and methods are used for characterizing one or more of the stage, grade, histological type, and nature of a COPD-related disease in a patient, it is desired that the marker or panel of markers is selected such that a positive result is obtained in at least about 20%, and in certain embodiments, at least about 40%, 60%, or 80%, and in substantially all patients afflicted with a COPD-related disease of the corresponding stage, grade, histological type, or nature. The marker or panel of markers invention can be selected such that a positive predictive value of greater than about 10% is obtained for the general population (in a non-limiting example, coupled with an assay specificity greater than 80%).

When a plurality of markers are used in the compositions, kits, and methods, the level of expression of each marker in a patient sample can be compared with the normal level of expression of each of the plurality of markers in non-COPD samples of the same type, either in a single reaction mixture (i.e. using reagents, such as different fluorescent probes, for each marker) or in individual reaction mixtures corresponding to one or more of the markers. In one embodiment, a significantly increased level of expression of more than one of the plurality of markers in the sample, relative to the corresponding normal levels, is an indication that the patient is afflicted with a COPD-related disease. When a plurality of markers is used, 2, 3, 4, 5, 8, 10, 12, 15, 20, 30, or 50 or more individual markers can be used; in certain embodiments, the use of fewer markers may be desired.

In order to maximize the sensitivity of the compositions, kits, and methods (i.e. by interference attributable to cells of non-respiratory system origin in a patient sample), it is desirable that the marker used therein be a marker which has a restricted tissue distribution, e.g., normally not expressed in a non-respiratory system tissue.

It is recognized that the compositions, kits, and methods will be of particular utility to patients having an enhanced risk of developing a COPD-related disease and their medical advisors. Patients recognized as having an enhanced risk of developing a COPD-related disease include, for example, patients having a familial history of a COPD-related disease, and smokers.

The level of expression of a marker in normal human respiratory system tissue can be assessed in a variety of ways. In one embodiment, this normal level of expression is assessed by assessing the level of expression of the marker in a portion of respiratory system cells which appear to be normal and by comparing this normal level of expression with the level of expression in a portion of the respiratory system cells which is suspected of being abnormal. Alternately, and particularly as further information becomes available as a result of routine performance of the methods described herein, population-average values for normal expression of the markers may be used. In other embodiments, the ‘normal’ level of expression of a marker may be determined by assessing expression of the marker in a patient sample obtained from a non-COPD-afflicted patient, from a patient sample obtained from a patient before the suspected onset of a COPD-related disease in the patient, from archived patient samples, and the like.

There is also provided herein compositions, kits, and methods for assessing the presence of COPD-related disease cells in a sample (e.g. an archived tissue sample or a sample obtained from a patient). These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions, in the kits, or the methods used to assess levels of marker expression in the sample.

The kits are useful for assessing the presence of COPD-related disease cells (e.g. in a sample such as a patient sample). The kit comprises a plurality of reagents, each of which is capable of binding specifically with a marker nucleic acid or protein. Suitable reagents for binding with a marker protein include antibodies, antibody derivatives, antibody fragments, and the like. Suitable reagents for binding with a marker nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kits may optionally comprise additional components useful for performing the methods described herein. By way of example, the kit may comprise fluids (e.g. SSC buffer) suitable for annealing complementary nucleic acids or for binding an antibody with a protein with which it specifically binds, one or more sample compartments, an instructional material which describes performance of the method, a sample of normal respiratory system cells, a sample of COPD-related disease cells, and the like.

There is also provided herein a method of making an isolated hybridoma which produces an antibody useful for assessing whether a patient is afflicted with a COPD-related disease. In this method, a protein or peptide comprising the entirety or a segment of a marker protein is synthesized or isolated (e.g. by purification from a cell in which it is expressed or by transcription and translation of a nucleic acid encoding the protein or peptide in vivo or in vitro). A vertebrate, for example, a mammal such as a mouse, rat, rabbit, or sheep, is immunized using the protein or peptide. The vertebrate may optionally (and preferably) be immunized at least one additional time with the protein or peptide, so that the vertebrate exhibits a robust immune response to the protein or peptide. Splenocytes are isolated from the immunized vertebrate and fused with an immortalized cell line to form hybridomas, using any of a variety of methods. Hybridomas formed in this manner are then screened using standard methods to identify one or more hybridomas which produce an antibody which specifically binds with the marker protein or a fragment thereof. There is also provided herein hybridomas made by this method and antibodies made using such hybridomas.

There is also provided herein a method of assessing the efficacy of a test compound for inhibiting COPD-related disease cells. As described above, differences in the level of expression of the markers correlate with the abnormal state of respiratory system cells. Although it is recognized that changes in the levels of expression of certain of the markers likely result from the abnormal state of respiratory system cells, it is likewise recognized that changes in the levels of expression of other of the markers induce, maintain, and promote the abnormal state of those cells. Thus, compounds which inhibit a COPD-related disease in a patient will cause the level of expression of one or more of the markers to change to a level nearer the normal level of expression for that marker (i.e. the level of expression for the marker in normal respiratory system cells).

This method thus comprises comparing expression of a marker in a first respiratory system cell sample and maintained in the presence of the test compound and expression of the marker in a second respiratory system cell sample and maintained in the absence of the test compound. A significantly reduced expression of a marker in the presence of the test compound is an indication that the test compound inhibits a COPD-related disease. The respiratory system cell samples may, for example, be aliquots of a single sample of normal respiratory system cells obtained from a patient, pooled samples of normal respiratory system cells obtained from a patient, cells of a normal respiratory system cell line, aliquots of a single sample of COPD-related disease cells obtained from a patient, pooled samples of COPD-related disease cells obtained from a patient, cells of a COPD-related disease cell line, or the like.

In one embodiment, the samples are COPD-related disease cells obtained from a patient and a plurality of compounds believed to be effective for inhibiting various a COPD-related diseases are tested in order to identify the compound which is likely to best inhibit the COPD-related disease in the patient.

This method may likewise be used to assess the efficacy of a therapy for inhibiting a COPD-related disease in a patient. In this method, the level of expression of one or more markers in a pair of samples (one subjected to the therapy, the other not subjected to the therapy) is assessed. As with the method of assessing the efficacy of test compounds, if the therapy induces a significantly lower level of expression of a marker then the therapy is efficacious for inhibiting a COPD-related disease. As above, if samples from a selected patient are used in this method, then alternative therapies can be assessed in vitro in order to select a therapy most likely to be efficacious for inhibiting a COPD-related disease in the patient.

As described above, the abnormal state of human respiratory system cells is correlated with changes in the levels of expression of the markers. There is also provided a method for assessing the harmful potential of a test compound. This method comprises maintaining separate aliquots of human respiratory system cells in the presence and absence of the test compound. Expression of a marker in each of the aliquots is compared. A significantly higher level of expression of a marker in the aliquot maintained in the presence of the test compound (relative to the aliquot maintained in the absence of the test compound) is an indication that the test compound possesses a harmful potential. The relative harmful potential of various test compounds can be assessed by comparing the degree of enhancement or inhibition of the level of expression of the relevant markers, by comparing the number of markers for which the level of expression is enhanced or inhibited, or by comparing both. Various aspects are described in further detail in the following subsections.

Isolated Proteins and Antibodies

One aspect pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein. In certain embodiments, useful proteins are substantially identical (e.g., at least about 40%, and in certain embodiments, 50%, 60%, 70%, 80%, 90%, 95%, or 99%) to one of these sequences and retain the functional activity of the corresponding naturally-occurring marker protein yet differ in amino acid sequence due to natural allelic variation or mutagenesis.

In addition, libraries of segments of a marker protein can be used to generate a variegated population of polypeptides for screening and subsequent selection of variant marker proteins or segments thereof.

Predictive Medicine

There is also provided herein uses of the animal models and markers in the field of predictive medicine in which diagnostic assays, prognostic assays, pharmacogenomics, and monitoring clinical trials are used for prognostic (predictive) purposes to thereby treat an individual prophylactically. Accordingly, there is also provided herein diagnostic assays for determining the level of expression of one or more marker proteins or nucleic acids, in order to determine whether an individual is at risk of developing a COPD-related disease. Such assays can be used for prognostic or predictive purposes to thereby prophylactically treat an individual prior to the onset of the COPD-related disease.

In another aspect, the methods are useful for at least periodic screening of the same individual to see if that individual has been exposed to chemicals or toxins that change his/her expression patterns.

Yet another aspect pertains to monitoring the influence of agents (e.g., drugs or other compounds administered either to inhibit a COPD-related disease or to treat or prevent any other disorder (e.g., in order to understand any respiratory system effects that such treatment may have) on the expression or activity of a marker in clinical trials.

Pharmacogenomics

The markers are also useful as pharmacogenomic markers. As used herein, a “pharmacogenomic marker” is an objective biochemical marker whose expression level correlates with a specific clinical drug response or susceptibility in a patient. The presence or quantity of the pharmacogenomic marker expression is related to the predicted response of the patient and more particularly the patient's tumor to therapy with a specific drug or class of drugs. By assessing the presence or quantity of the expression of one or more pharmacogenomic markers in a patient, a drug therapy which is most appropriate for the patient, or which is predicted to have a greater degree of success, may be selected.

Monitoring Clinical Trials

Monitoring the influence of agents (e.g., drug compounds) on the level of expression of a marker can be applied not only in basic drug screening, but also in clinical trials. For example, the effectiveness of an agent to affect marker expression can be monitored in clinical trials of subjects receiving treatment for a COPD-related disease. In one non-limiting embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly.

For example, increased expression of the marker gene(s) during the course of treatment may indicate ineffective dosage and the desirability of increasing the dosage. Conversely, decreased expression of the marker gene(s) may indicate efficacious treatment and no need to change dosage.

Electronic Apparatus Readable Media and Arrays

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the marker information of the present invention on the electronic apparatus readable medium. Any number of data processor structuring formats (e.g., text file or database) may be employed in order to obtain or create a medium having recorded thereon the markers. By providing the markers in readable form, one can routinely access the marker sequence information for a variety of purposes. For example, one skilled in the art can use the nucleotide or amino acid sequences in readable form to compare a target sequence or target structural motif with the sequence information stored within the data storage means. Search means are used to identify fragments or regions of the sequences which match a particular target sequence or target motif.

Thus, there is also provided herein a medium for holding instructions for performing a method for determining whether a subject has a COPD-related disease or a pre-disposition to a COPD-related disease, wherein the method comprises the steps of determining the presence or absence of a marker and based on the presence or absence of the marker, determining whether the subject has a COPD-related disease or a pre-disposition to a COPD-related disease and/or recommending a particular treatment for a COPD-related disease or pre-COPD-related disease condition.

There is also provided herein an electronic system and/or in a network, a method for determining whether a subject has a COPD-related disease or a pre-disposition to a COPD-related disease associated with a marker wherein the method comprises the steps of determining the presence or absence of the marker, and based on the presence or absence of the marker, determining whether the subject has a COPD-related disease or a pre-disposition to a COPD-related disease, and/or recommending a particular treatment for the COPD-related disease or pre-COPD-related disease condition. The method may further comprise the step of receiving phenotypic information associated with the subject and/or acquiring from a network phenotypic information associated with the subject.

Also provided herein is a network, a method for determining whether a subject has a COPD-related disease or a pre-disposition to a COPD-related disease associated with a marker, the method comprising the steps of receiving information associated with the marker, receiving phenotypic information associated with the subject, acquiring information from the network corresponding to the marker and/or a COPD-related disease, and based on one or more of the phenotypic information, the marker, and the acquired information, determining whether the subject has a COPD-related disease or a pre-disposition to a COPD-related disease. The method may further comprise the step of recommending a particular treatment for the COPD-related disease or pre-COPD-related disease condition.

There is also provided herein a business method for determining whether a subject has a COPD-related disease or a pre-disposition to a COPD-related disease, the method comprising the steps of receiving information associated with the marker, receiving phenotypic information associated with the subject, acquiring information from the network corresponding to the marker and/or a COPD-related disease, and based on one or more of the phenotypic information, the marker, and the acquired information, determining whether the subject has a COPD-related disease or a pre-disposition to a COPD-related disease. The method may further comprise the step of recommending a particular treatment for the COPD-related disease or pre-COPD-related disease condition.

There is also provided herein an array that can be used to assay expression of one or more genes in the array. In one embodiment, the array can be used to assay gene expression in a tissue to ascertain tissue specificity of genes in the array. In this manner, up to about 7000 or more genes can be simultaneously assayed for expression. This allows a profile to be developed showing a battery of genes specifically expressed in one or more tissues.

In addition to such qualitative determination, there is provided herein the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the method provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of a COPD-related disease, progression of a COPD-related disease, and processes, such as cellular transformation associated with a COPD-related disease.

The array is also useful for ascertaining the effect of the expression of a gene or the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

Surrogate Markers

The markers may serve as surrogate markers for one or more disorders or disease states or for conditions leading up to a COPD-related disease state. As used herein, a “surrogate marker” is an objective biochemical marker which correlates with the absence or presence of a disease or disorder, or with the progression of a disease or disorder. The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies, or when an assessment of disease progression is desired before a potentially dangerous clinical endpoint is reached.

The markers are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker transcription or expression, the amplified marker may be in a quantity which is more readily detectable than the drug itself. Also, the marker may be more easily detected due to the nature of the marker itself; for example, using the methods described herein, antibodies may be employed in an immune-based detection system for a protein marker, or marker-specific radiolabeled probes may be used to detect a mRNA marker. Furthermore, the use of a pharmacodynamic marker may offer mechanism-based prediction of risk due to drug treatment beyond the range of possible direct observations.

Experimental Protocol

The method of testing for COPD-related diseases comprises, for example measuring the expression level of each marker gene in a biological sample from a subject (i.e., heavy smokers with and without COPD) over time and comparing the level with that of the marker gene in a control biological sample.

When the marker gene is one of the genes described herein and the expression level is differentially expressed (for examples, higher or lower than that in the control), the subject is judged to be affected with a COPD-related disease. When the expression level of the marker gene falls within the permissible range, the subject is unlikely to be affected with a COPD-related disease.

The standard value for the control may be pre-determined by measuring the expression level of the marker gene in the control, in order to compare the expression levels. For example, the standard value can be determined based on the expression level of the above-mentioned marker gene in the control. For example, in certain embodiments, the permissible range is taken as ±2S.D. based on the standard value. Once the standard value is determined, the testing method may be performed by measuring only the expression level in a biological sample from a subject and comparing the value with the determined standard value for the control.

Expression levels of marker genes include transcription of the marker genes to mRNA, and translation into proteins. Therefore, one method of testing for a COPD-related disease is performed based on a comparison of the intensity of expression of mRNA corresponding to the marker genes, or the expression level of proteins encoded by the marker genes.

The measurement of the expression levels of marker genes in the testing for a COPD-related disease can be carried out according to various gene analysis methods. Specifically, one can use, for example, a hybridization technique using nucleic acids that hybridize to these genes as probes, or a gene amplification technique using DNA that hybridize to the marker genes as primers.

The probes or primers used for the testing can be designed based on the nucleotide sequences of the marker genes. The identification numbers for the nucleotide sequences of the respective marker genes are shown in various Tables herein.

Further, it is to be understood that genes of higher animals generally accompany polymorphism in a high frequency. There are also many molecules that produce isoforms comprising mutually different amino acid sequences during the splicing process. Any gene associated with a COPD-related disease that has an activity similar to that of a marker gene is included in the marker genes, even if it has nucleotide sequence differences due to polymorphism or being an isoform.

It is also to be understood that the marker genes can include homologs of other species in addition to humans. Thus, unless otherwise specified, the expression “marker gene” refers to a homolog of the marker gene unique to the species or a foreign marker gene which has been introduced into an individual. Also, it is to be understood that a “homolog of a marker gene” refers to a gene derived from a species other than a human, which can hybridize to the human marker gene as a probe under stringent conditions. Such stringent conditions are known to one skilled in the art who can select an appropriate condition to produce an equal stringency experimentally or empirically.

A polynucleotide comprising the nucleotide sequence of a marker gene or a nucleotide sequence that is complementary to the complementary strand of the nucleotide sequence of a marker gene and has at least 15 nucleotides, can be used as a primer or probe. Thus, a “complementary strand” means one strand of a double stranded DNA with respect to the other strand and which is composed of A:T (U for RNA) and G:C base pairs. In addition, “complementary” means not only those that are completely complementary to a region of at least 15 continuous nucleotides, but also those that have a nucleotide sequence homology of at least 40% in certain instances, 50% in certain instances, 60% in certain instances, 70% in certain instances, at least 80%, 90%, and 95% or higher. The degree of homology between nucleotide sequences can be determined by an algorithm, BLAST, etc.

Such polynucleotides are useful as a probe to detect a marker gene, or as a primer to amplify a marker gene. When used as a primer, the polynucleotide comprises usually 15 bp to 100 bp, and in certain embodiments 15 bp to 35 bp of nucleotides. When used as a probe, a DNA comprises the whole nucleotide sequence of the marker gene (or the complementary strand thereof), or a partial sequence thereof that has at least 15 bp nucleotides. When used as a primer, the 3′ region must be complementary to the marker gene, while the 5′ region can be linked to a restriction enzyme-recognition sequence or a tag.

“Polynucleotides” may be either DNA or RNA. These polynucleotides may be either synthetic or naturally-occurring. Also, DNA used as a probe for hybridization is usually labeled. Those skilled in the art readily understand such labeling methods. Herein, the term “oligonucleotide” means a polynucleotide with a relatively low degree of polymerization. Oligonucleotides are included in polynucleotides.

Tests for a COPD-related disease using hybridization techniques can be performed using, for example, Northern hybridization, dot blot hybridization, or the DNA microarray technique. Furthermore, gene amplification techniques, such as the RT-PCR method may be used. By using the PCR amplification monitoring method during the gene amplification step in RT-PCR, one can achieve a more quantitative analysis of the expression of a marker gene.

In the PCR gene amplification monitoring method, the detection target (DNA or reverse transcript of RNA) is hybridized to probes that are labeled with a fluorescent dye and a quencher which absorbs the fluorescence. When the PCR proceeds and Taq polymerase degrades the probe with its 5′-3′ exonuclease activity, the fluorescent dye and the quencher draw away from each other and the fluorescence is detected. The fluorescence is detected in real time. By simultaneously measuring a standard sample in which the copy number of a target is known, it is possible to determine the copy number of the target in the subject sample with the cycle number where PCR amplification is linear. Also, one skilled in the art recognizes that the PCR amplification monitoring method can be carried out using any suitable method.

The method of testing for a COPD-related disease can be also carried out by detecting a protein encoded by a marker gene. Hereinafter, a protein encoded by a marker gene is described as a “marker protein.” For such test methods, for example, the Western blotting method, the immunoprecipitation method, and the ELISA method may be employed using an antibody that binds to each marker protein.

Antibodies used in the detection that bind to the marker protein may be produced by any suitable technique. Also, in order to detect a marker protein, such an antibody may be appropriately labeled. Alternatively, instead of labeling the antibody, a substance that specifically binds to the antibody, for example, protein A or protein G, may be labeled to detect the marker protein indirectly. More specifically, such a detection method can include the ELISA method.

A protein or a partial peptide thereof used as an antigen may be obtained, for example, by inserting a marker gene or a portion thereof into an expression vector, introducing the construct into an appropriate host cell to produce a transformant, culturing the transformant to express the recombinant protein, and purifying the expressed recombinant protein from the culture or the culture supernatant. Alternatively, the amino acid sequence encoded by a gene or an oligopeptide comprising a portion of the amino acid sequence encoded by a full-length cDNA are chemically synthesized to be used as an immunogen.

Furthermore, a test for a COPD-related disease can be performed using as an index not only the expression level of a marker gene but also the activity of a marker protein in a biological sample. Activity of a marker protein means the biological activity intrinsic to the protein. Various methods can be used for measuring the activity of each protein.

Even if a patient is not diagnosed as being affected with a COPD-related disease in a routine test in spite of symptoms suggesting these diseases, whether or not such a patient is suffering from a COPD-related disease can be easily determined by performing a test according to the methods described herein.

More specifically, in certain embodiments, when the marker gene is one of the genes described herein, an increase or decrease in the expression level of the marker gene in a patient whose symptoms suggest at least a susceptibility to a COPD-related disease indicates that the symptoms are primarily caused by bronchial asthma or a chronic obstructive pulmonary disease (COPD).

In addition, the tests are useful to determine whether a COPD-related disease is improving in a patient. In other words, the methods described herein can be used to judge the therapeutic effect of a treatment for a COPD-related disease. Furthermore, when the marker gene is one of the genes described herein, an increase or decrease in the expression level of the marker gene in a patient, who has been diagnosed as being affected by a COPD-related disease, implies that the disease has progressed more.

The severity and/or susceptibility to a COPD-related disease may also be determined based on the difference in expression levels. For example, when the marker gene is one of the genes described herein, the degree of increase in the expression level of the marker gene is correlated with the presence and/or severity of a COPD-related disease.

In another aspect, there is provided herein animal models for a COPD-related disease where the expression level of one or more marker genes or a gene functionally equivalent to the marker gene has been elevated in the animal model. A “functionally equivalent gene” as used herein generally is a gene that encodes a protein having an activity similar to a known activity of a protein encoded by the marker gene. A representative example of a functionally equivalent gene includes a counterpart of a marker gene of a subject animal, which is intrinsic to the animal.

The animal model for a COPD-related disease is useful for detecting physiological changes due to a COPD-related disease. In certain embodiments, the animal model is useful to reveal additional functions of marker genes and to evaluate drugs whose targets are the marker genes.

In one embodiment, an animal model for a COPD-related disease can be created by controlling the expression level of a counterpart gene or administering a counterpart gene. The method can include creating an animal model for a COPD-related disease by controlling the expression level of a gene selected from the group of genes described herein. In another embodiment, the method can include creating an animal model for a COPD-related disease by administering the protein encoded by a gene described herein, or administering an antibody against the protein. It is to be also understood, that in certain other embodiments, the marker can be over-expressed such that the marker can then be measured using appropriate methods.

In another embodiment, an animal model for a COPD-related disease can be created by introducing a gene selected from such groups of genes, or by administering a protein encoded by such a gene. Such counterpart genes or proteins can be introduced/administered to mice, because they are derived from mice.

In another embodiment, a COPD-related disease can be induced by suppressing the expression of a gene selected from such groups of genes or the activity of a protein encoded by such a gene. An antisense nucleic acid, a ribozyme, or an RNAi can be used to suppress the expression. The activity of a protein can be controlled effectively by administering a substance that inhibits the activity, such as an antibody.

The animal model is useful to elucidate the mechanism underlying a COPD-related disease and also to test the safety of compounds obtained by screening. For example, when an animal model develops the symptoms of a COPD-related disease, or when a measured value involved in a certain a COPD-related disease alters in the animal, a screening system can be constructed to explore compounds having activity to alleviate the disease.

As used herein, the expression “an increase in the expression level” refers to any one of the following: where a marker gene introduced as a foreign gene is expressed artificially; where the transcription of a marker gene intrinsic to the subject animal and the translation thereof into the protein are enhanced; or where the hydrolysis of the protein, which is the translation product, is suppressed. As used herein, the expression “a decrease in the expression level” refers to either the state in which the transcription of a marker gene of the subject animal and the translation thereof into the protein are inhibited, or the state in which the hydrolysis of the protein, which is the translation product, is enhanced. The expression level of a gene can be determined, for example, by a difference in signal intensity on a DNA chip. Furthermore, the activity of the translation product—the protein—can be determined by comparing with that in the normal state.

It is also within the contemplated scope that the animal model can include transgenic animals, including, for example animals where a marker gene has been introduced and expressed artificially; marker gene knockout animals; and knock-in animals in which another gene has been substituted for a marker gene. A transgenic animal, into which an antisense nucleic acid of a marker gene, a ribozyme, a polynucleotide having an RNAi effect, or a DNA functioning as a decoy nucleic acid or such has been introduced, can be used as the transgenic animal. Such transgenic animals also include, for example, animals in which the activity of a marker protein has been enhanced or suppressed by introducing a mutation(s) into the coding region of the gene, or the amino acid sequence has been modified to become resistant or susceptible to hydrolysis. Mutations in an amino acid sequence include substitutions, deletions, insertions, and additions. In addition, the expression itself of a marker gene can be controlled by introducing a mutation(s) into the transcriptional regulatory region of the gene. Those skilled in the art understand such amino acid substitutions. Also, the number of amino acids that are mutated is not particularly restricted, as long as the activity is maintained. Normally, it is within 50 amino acids, in certain non-limiting embodiments, within 30 amino acids, within 10 amino acids, or within 3 amino acids. The site of mutation may be any site, as long as the activity is maintained.

In yet another aspect, there is provided herein screening methods for candidate compounds for therapeutic agents to treat a COPD-related disease. One or more marker genes are selected from the group of genes described herein. A therapeutic agent for a COPD-related disease can be obtained by selecting a compound capable of increasing or decreasing the expression level of the marker gene(s). It is to be understood that the expression “a compound that increases the expression level of a gene” refers to a compound that promotes any one of the steps of gene transcription, gene translation, or expression of a protein activity. On the other hand, the expression “a compound that decreases the expression level of a gene”, as used herein, refers to a compound that inhibits any one of these steps.

In particular aspects, the method of screening for a therapeutic agent for a COPD-related disease can be carried out either in vivo or in vitro. This screening method can be performed, for example, by (1) administering a candidate compound to an animal subject; (2) measuring the expression level of a marker gene(s) in a biological sample from the animal subject; or (3) selecting a compound that increases or decreases the expression level of a marker gene(s) as compared to that in a control with which the candidate compound has not been contacted.

In still another aspect, there is provided herein a method to assess the efficacy of a candidate compound for a pharmaceutical agent on the expression level of a marker gene(s) by contacting an animal subject with the candidate compound and monitoring the effect of the compound on the expression level of the marker gene(s) in a biological sample derived from the animal subject. The variation in the expression level of the marker gene(s) in a biological sample derived from the animal subject can be monitored using the same technique as used in the testing method described above. Furthermore, based on the evaluation, a candidate compound for a pharmaceutical agent can be selected by screening.

In a particular aspect, there is provided herein a lipopolysaccharide (LPS) compromised animal model as a model for the investigation of COPD-related diseases. The co-exposure of smoke and LPS heighten the inflammatory response and other pulmonary lesions in mice exposed to these two irritants.

Also provided is a testing method that includes a co-exposure regimen of cigarette smoke and LPS that induces consistent and heightened inflammatory responses and ultimately, following a longer period of exposure to cigarette smoke and LPS, produce definitive morphologic evidence of histopathologic changes typical of COPD (the phenotype) and morphometric evidence of changes within a shorter time than with cigarette smoke alone. In the testing method there is provided a LPS inhalation exposure regimen that allows repeated nose-only inhalation exposures to the animal model without causing acute moribundity or mortality. In certain embodiments, the morphological evidence of change is seen in alveolar lumens and septa diagnostic of COPD using lung morphometry.

In another aspect, there is provided a system for gene expression profiling which identifies genes and function modules related to COPD-related disease pathogenesis. These genes and function modules are useful as markers to monitor COPD-related disease progression.

The animal models were exposed to HEPA-filtered air (sham control group), cigarette smoke (smoke group), LPS (LPS group), or Smoke plus LPS (Smoke+LPS group) by nose-only inhalation. Lungs were collected at the end of the 3 wk exposure and processed for microarray analysis. Clustering and network analysis showed decreased heat shock response and chaperone activity, increased immune and inflammatory response, and increased mitosis in all three exposed groups.

The number of genes and function modules/networks associated with inflammation was reduced in the Smoke+LPS group compared to the LPS group, an indication of immune suppression by the smoke co-exposure. It is to be noted that the animal models exposed to Smoke+LPS showed more macrophage infiltration which looked more like what would be seen in chronic exposure; when in fact, such macrophage infiltration occurred only after 3 week exposure.

The most up-regulated gene in the Smoke group, MMP12, is a matrix metalloproteinase that preferentially degrades elastin and has been implicated in COPD development.

NOXO1 positively regulates the expression of a subunit of NADPH oxidase (NOX1), a major source of reactive oxygen species and may play an important role in the pathogenesis of COPD. The highest up-regulated gene in the Smoke group is serum amyloid A1, MMP 12 is the second highest.

Serum amyloid A1, which is an acute phase systemic inflammation marker and can be induced by LPS exposure, is significantly up-regulated in the LPS group (>100-fold), Smoke+LPS group (>100-fold), and the Smoke group (20-fold). Serum amyloid A3 is highly up-regulated (40-fold) in the Smoke+LPS group.

MARCO is a scavenger receptor expressed in macrophages and may play a significant role in LPS-induced inflammatory response.

The chemokine (C-X-C motif) ligand (CXCL9) is highly regulated with LPS and Smoke+LPS, and is a secreted inflammatory marker that is poorly characterized, although expression of which remains elevated during chronic infection and is believed to be involved in T cell trafficking.

### EXAMPLE I

### Maker Capability—Characterization of Inflammatory Responses in Mice Upon LPS and/or Cigarette Smoke Exposure

Test System—Test Animals were male AKR/J mice. The test system information is summarized in TABLE 1 below.

TABLE 1

| | |
|---|---|
| Species | Mus musculus |
| Strain | AKR/J; referred as NJ in this report |
| Source | Jackson Lab, Bar Harbor, ME |
| Number for Study | 90 mice |
| Total Number and Date | 250 males received Feb. 01, 2005 |
| Age | −9-13 weeks at exposure start |
| Identification | Tail tattoo (AIMS, Inc.; Piscataway, NJ); |
| | Placement within the chamber cage unit |
| Exposure Day 1 | Feb. 03, 2005 |
| Terminal Sacrifice/Necropsy | Mar. 18, 2005 |

Acclimatization to Restrain Tubes

Prior to exposure, the animals were placed in the nose-only restraint tubes for acclimatization. The nose-only exposure tubes have a number of features to minimize stress, such as body ventilation holes and channels to remove urine and feces.

Group Assignment

The Xybion PATH/TOX SYSTEM™ (Xybion Medical Systems; Cedar Knolls, N.J.) was used for randomization and exposure group assignment. For each phase, the animals were assigned to exposure groups using body weight as a blocking variable to ensure that there were no statistically significant differences in initial group mean body weights. The weight distribution range of the animals selected for the study was no more than ±20% from the mean body weight of the animals available for the study.

Cigarette Smoke+LPS 3-Week Exposures

Animals were exposed via nose-only inhalation to one of the following exposure regimens for a total of −6 hr/day, 5 days/week for 3 consecutive weeks, as follows: LPS only (exposed to filtered air for the first 5 hr, followed by LPS exposure at target dose for 1 hr/day, twice per week); cigarette smoke only (250 μg WTPM/L for 5 hr/day, followed by filtered-air exposure for the last 1 hr/day); or LPS/cigarette smoke (cigarette smoke at 250 μg/L for 5 hr/day, 5 days/week, followed by LPS exposure for 1 hr/day, twice per week).

The sham control was exposed to high-efficiency particulate air (HEPA)-filtered humidified air via nose-only inhalation for 6 hr/day, 5 days/week, for 3 weeks, as shown in TABLE 2 below:

TABLE 2
Study Design

| | | | Subgroup 2: | |
|---|---|---|---|---|
| | | | Histopathology & | |
| | | Sub- | Immunohisto- | |
| Exposure | | group 1: | chemistry/ | Subgroup 3: |
| Regimen | Group* | BAL | Proteomics | Genomics |
| Control | Sham | 6 | 6 | 6 |
| Exposure | E1: LPS | 6 | 6 | 6 |
| | E2: Cigarette | 6 | 6 | 6 |
| | E3: Smoke + LPS | 6 | 6 | 6 |

| | |
|---|---|
| Total | 72 |

Subgroup 1; Respiratory Physiology, Bleeding, & BAL

Selected animals (5 out of 6/group) were subjected to respiratory physiology measurement during Week 3 for 10-min preexposure and the first 30-min exposure period. After the respiratory physiology was completed, animals were returned to the exposure to complete the rest of exposure regimen. Immediately after the last exposure, blood samples were collected (5 mice/group) in the exposure room for Sham control, E2 (cigarette smoke exposure only) and E3 (cigarette Smoke+LPS exposure). Animals were returned to the cage until subjected to BAL. Blood COHb concentrations were determined using an OSM3 Hemoximeter. The blood remaining in each sample was centrifuged for collection of plasma. Plasma samples were stored at about 70° C. until analyzed for plasma nicotine and cotinine concentrations using gas chromatography/mass spectrometry. The following morning after the last exposure, animals were euthanized with an IP injection of pentobarbital and then the lungs removed and subjected to BAL. BALF was analyzed for LPS and clinical chemistry/cytology.

Subgroup 2: Lung Histopathology/Immunohistochemistry, & Proteomics

The following morning after the last exposure, animals were euthanized with an IP injection of pentobarbital and blood samples collected via vena cava. The whole lung was weighed before being tied for division. The right lung was dedicated for histopathological assessment/immunohistochemistry analyses, and the left lung lobe was weighed before snap-freezing. For proteomics, left lung lobe was collected, weighed, and put into a tube containing approximately two volumes of 50 mM ammonium bicarbonate/detergent as rapidly as possible. The container was then flash frozen and stored at −70° C. prior to analysis. Only 5/group were analyzed, the remaining 1/group was stored for future analysis.

Subgroup 3: Lung Genomics

The following morning after the last exposure, animals were euthanized with an IP injection of pentobarbital. No blood sampling was done. The lung was removed immediately and divided for genomics (right) and histopathology (left). For genomics, the right lobes of lung were put in RNA later immediately and shipped on ice to BCO for RNA isolation and microarray analysis.

Body Weights, Mortality, and Clinical Observations

Animals were observed for moribundity and mortality at least once daily throughout the study. Body weights and clinical signs were collected manually and entered into the Xybion PATH/TOX SYSTEM™.

Individual body weights were recorded at the time of randomization, prior to exposure on Day 1, approximately weekly thereafter, and prior to scheduled sacrifice. Individual clinical observations were recorded weekly and prior to scheduled sacrifice.

Respiratory Function Measurements

The Buxco® system was used to measure respiratory function parameters once on Week 3 (exposed and sham groups; 5 animals/group from Subgroup 1). Respiratory function was monitored during the 10 min pre-exposure and for 30-min exposure period. After the measurement, animals were returned to the exposure unit for the remainder of exposure duration.

The digitized respiratory flow and calculated respiratory parameters (i.e., tidal volume, respiratory rate, and minute volume) were monitored from each animal via plethysmography.

All respiratory parameters were averaged for each 1-min portion of the measurement period. The mean and standard deviation of the respiratory parameters were calculated for the 10 min pre-exposure and 30-min exposure period.

Necropsy

A partial necropsy was performed on all Subgroup 1, 2 and 3 mice. Mice were anesthetized with sodium pentobarbital, then euthanized by exsanguination. Findings were recorded on Individual Animal Necropsy Record (IANR) forms. Necropsies included an external examination of the animal and all body orifices and examination and fixation as per study design of all the lung tissues. Carcasses were discarded.

In yet another aspect, there is provided a useful diagnostic tool wherein the addition of LPS to Smoke exposure altered the Smoke- or LPS-associated inflammatory responses in tissue/BALF and is used in developing a LPS-compromised mouse COPD model.

The Smoke and/or LPS exposure regimens were determined from a range-finding study to assure no acute mortality or moribundity:

Range finding-1: Single LPS Exposure; Single 1 hr inhalation at ˜5-10 ug LPS/mouse & 24 sacrifice. Results: Acute toxicity/severe lung inflammatory response

Range finding-2: Five day LPS Exposure; Repeated 1 hr inhalation at ˜2.5 ug LPS/mouse, 5 consecutive days 2 hr post exposure sacrifice. Results: Moderate lung inflammatory response, potentially too severe for repeated exposure.

Smoke/LPS 3-Wk Inhalation Exposure; Cigarettes smoke, 5 hr/day/wk at 250 ug/L WTPM; LPS, 1 hr/day twice/wk at ˜0.5 ug LPS/mouse.

After the last exposure, blood was collected and analyzed for COHb and nicotine. The following morning, mice were necropsied and lungs collected for BAL, histopathology/immunohistology (TUNEL assay), transcriptomics or proteomics.

LPS aerosol was generated, chemically monitored, and exposed to mice via nose-only inhalation in a respirable size (TABLE 3 below).

TABLE 3

| | Cigarette | | Particle | | | | | |
|---|---|---|---|---|---|---|---|---|
| | smoke | | Size | | | | | Particle |
| | conc. | RAM | Distrib'n | | Avg | LAL: | RAM | Size |
| | (μm | variability | Smoke | CO | Butt | LSP | variability | Distrib'n |
| | WTPM/ | (Smoke) | MMAD | Conc. | Length | Conc. | (LSP) | MMAD |
| Metric | L) | (% RSD) | (μm) | (ppm) | (mm) | (μg/L) | (% RSD) | (μm) |
| Mean | 255 | 6.54 | 0.61 | 273 | 36 | 0.49 | 29.0 | 0.40 |
| SD | 7 | 2.4 | 0.01 | 6 | 0 | 0.21 | 4.2 | 0.09 |
| RSD | 2.9% | — | 2.3% | 2.2% | 1.1% | 42.3% | — | 223.3% |
| N | 52 | 13 | 2 | 13 | 13 | 24 | 6 | |

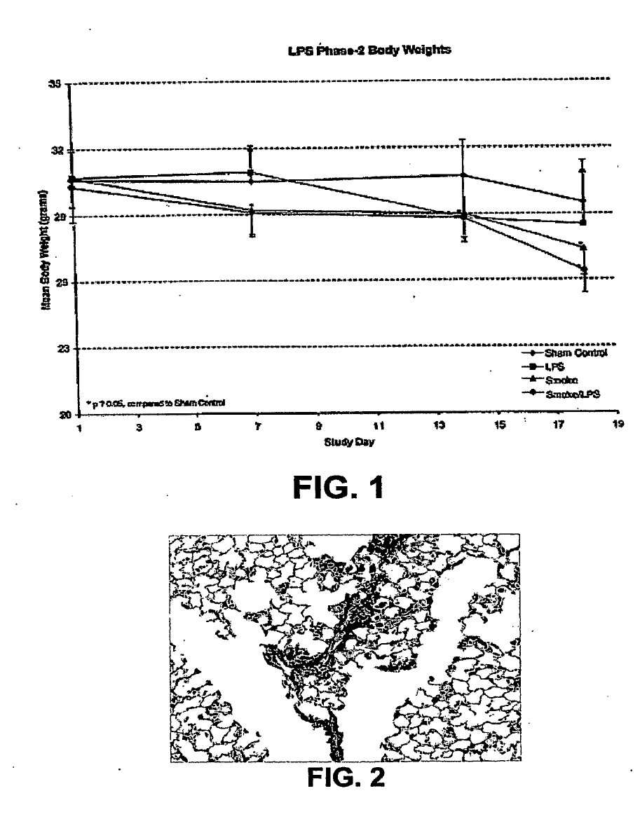

AKR/J mice were exposed to CS at 250 tg/L WTPM for 5 h, followed by LPS for 1 h without showing acute toxicity. There was clear suppression of body weight after 3 weeks of exposure to Smoke and Smoke+LPS. See FIG. 1.

BAL (Subgroup 1)

BAL was performed on isolated lungs by cannulating the trachea and washing the lungs six times with phosphate-buffered saline (PBS, pH 7.2; kept at room temperature) using a volume of approximately 1 mL/wash. Retrieved fluid was kept on ice.

The first two washes and the rest of washes were separately pooled and centrifuged at ˜1700 rpm for 10 min at −4° C. After centrifugation, the cell-free BALF (supernatant) from the first or the first two washes was measured for its approximate volume using a graduated tube and divided into containers as listed in the TABLE below. Supernatant from the rest of the washes was not analyzed. The BAL fluid (BALF) was used as shown in TABLE 4 below:

TABLE 4
BAL Fluid Use

| Number of | BALF Volume/ | | Sample |
|---|---|---|---|
| Tubes/animal | Tube (pL) | Markers | Prep |
| 1 | 200 | M1: LDH, NAG, protein | Fresh |
| 1 | 200 | M2: Cytokines | Frozen |
| 1 | 200 | M3: LPS analysis* | Frozen |

| | |
|---|---|
| Excess BALF stored frozen at −70 C. | Frozen |

Note:
lactate dehydrogenase (LDH), N-acetyl-B-D-glucosaminidase (NAG)
*Discussed in exposure & chemistry CSR. Cell pellets from all 6 washes were used for cytological evaluations (viability, cell count, cell differentials).

LDH, NAG, and Protein Analyses

BALF samples were analyzed fresh for LDH, NAG, and/or protein, using Hitachi methodologies (Roche Hitachi 912 System, Roche Diagnostic Corp: Indianapolis, Ind.).

Cytokine Assay

BALF samples were immediately frozen at −70° C. Samples were analyzed in triplicate for 19 cytokines [IL-1a, IL-113, IL-2, IL-3, IL-4, IL-5, IL-6, IL 10, IL-12p40, IL-12p70, IL-17, G-CSF, GM-CSF, IFN-y, KC, MIP-1 a, RANTES, TNF-a, Bio-Rad, Hercules, Calif.; TARC, R&D Systems, Minneapolis, Minn.] using the Bioplex suspension array system™ (Bio-Rad, Hercules, Calif.).

For this we separated the proteins in BAL fluid by electrophoresis using 10% 4 non-denaturing tris-glycine polyacrylamide gel containing 0.1% gelatin. After separation the proteases were allowed to digest the gelatin and were then stained with SimplyBlue™ SafeStain. Samples were separated on two gels and a positive control (BAL from an animal receiving a very high LPS dose; lane 12 on each gel labeled RF 7, 5 uL) was used to indicate that similar amounts of proteolytic activity were detected on each gel; this indicates that the intensity of the bands can be compared between gels.

Histopathology and Apoptosis/Terminal Deoxynucleotidyl Transferase Mediated dUTP Nick Ended Labeling (TUNEL) Assay (Subgroup 2)

Histopathology evaluation of the lung was performed on Subgroup 2 animals. An immunohistochemical method was developed to perform the TUNEL Assay which labels positive cells that have undergone apoptosis

Statistics

Statistical evaluations included the one-way analysis of variance (ANOVA) followed by Bartlett's test of homogeneity of variance. If the variances were homogenous, then Dunnett's t-test (modified t-test) was performed. If the variances were non-homogenous, then a Cochran & Cox modified t-test was used. Statistical analysis was performed, comparing the sham control to the exposure groups.

Results

Carboxyhemoglobin (COHb)

Results are summarized in TABLE 5. Carboxyhemoglobin was elevated in the Smoke and Smoke+LPS groups. However, the mean percent COHb in the Smoke+LPS group was −½ that of the Smoke group.

TABLE 5
Carboxyhemoglobin (Mean ± SD; n = 5)

| Period | Group | MV | COHb (% |
|---|---|---|---|
| Baseline | Sham control | 63.5 ± 7.7 | NA |
| | LPS | 76.7 ± 11.3 | NA |
| | Smoke | 49.6 = 13.4 | NA |
| | Smoke + LPS | 49.2 ± 8.3 | NA |
| Exposure | Sham control | 55.4 ± 5.5 | 0.7 0.2 |
| | LPS | 61.3 ± 6.6 | NA |
| | Smoke | 11.2 ± 1.9 | 13.6 ± 4.7 |
| | Smoke + LPS | 13.1 ± 7.8 | 7.2 ± 2.5 |

Results for BAL are presented in Tables 6, 7 and 8 below.

Cytology

All exposure groups had elevated total cell, macrophage (PAM), neutrophils (PMN), and lymphocyte (not increased in LPS group) counts. The total cell, PAM, and PMN counts in smoke exposed animals were lower then LPS or Smoke+LPS groups. LPS exposed mice had the highest number of PMNs while Smoke+LPS exposed animals had the greatest increase in PAMs.

TABLE 6
BALF Cytology (Mean ± SD; n = 6)

| Exposure | | | | |
|---|---|---|---|---|
| Regimen | Sham Control | LPS (E1) | Smoke (E2) | Smoke + LPS (E3) |
| Total Cell | 703 ± 229 | 3982 ± 1043* | 1443 ± 371* | 3514 ± 1173* |
| % Dead | 5.2 ± 1.9 | 3.4 ± 2.0 | 5.5 ± 2.8 | 4.1 ± 1.6 |
| PAMs | 696.3 ± 223.0 | 1670.9 ± 508.0* | 1007.5 ± 250.6 | 2902.6 ± 1094.4* |
| PMNs | 7.0 ± 11.3 | 2310.8 ± 822.9″ | 429.2 ± 238.3* | 606.9 ± 223.1* |
| Lymphs | 0.0 ± 0.0 | 0.0 ± 0.0 | 6.6 ± 9.7 | 4.7 ± 5.1 |

*p .05 Dunnett's t-test of significance, compared to sham control

Chemistry

LDH, protein, and NAG were increased in all exposure groups. The Smoke+LPS group had the greatest increase in LDH (Smoke+LPS>smoke>LPS>sham), while the smoke group had the greatest increase in NAG (smoke>Smoke+LPS>LPS>sham).

TABLE 7
BALF Chemistries (Mean ± SD; n = 6)

| Exposure | | | Smoke | Smoke + |
|---|---|---|---|---|
| Regimen | Sham Control | LPS (E1) | (E2) | LPS (E3) |
| LDH (U/L) | 29 ± 16 | 65 ± 13* | 77 ± 8* | 86 ± 32* |
| Protein (mg/dL) | 4.2 ± 1.5 | 7.5 ± 2.2* | 7.3 ± 1.6* | 8.2 ± 4.8 |
| NAG (U/L) | 0.5 ± 0.1 | 0.8 ± 0.3 | 3.2 ± 0.4* | 1.1 ± 0.4 |

*p .05 Dunnett's t-test of significance, compared to sham control ^{a}N = 5

Cytokines

Significant increases in IL-1a, IL-1R, TNF-a, IL-6, IL-12p40, G-CSF, MIP-1 a, RANTES, and TARC were seen in LPS exposed animals compared to the sham control.

Significant increases in IL-1a, IL-12p40, G-CSF, KC, MIP-1a, and TARC were present in smoke exposed animals compared to the sham control.

Significant increases in IL-12p40, G-CSF, and RANTES were present in Smoke+LPS exposed animals compared to the sham control. IL-2, IL-5, and IL12p70 were significantly reduced.

TABLE 8
BAL Cytokines (Mean ± SD; N = 6)

| Exposure | | | | |
|---|---|---|---|---|
| Regimen | Sham Control^{a} | LPS (E1) | Smoke (E2) | Smoke + LPS (E3) |
| IL-1 R | 0.27 ± 0.59 | 23.09 ± 15.38* | 0.27 ± 0.50 | 1.10 ± 1.88 |
| IL-2 | 2.18 ± 11.12 | 12.61 ± 7.32 | 13.28 ± 4.54 | 6.71 ± 5.12* |
| IL-4 | 1.17 ± 0.66 | 1.06 ± 1.21 | 0.45 ± 0.87 | 0.24 ± 0.54 |
| IL-5 | 6.32 ± 2.05 | 6.86 ± 4.28 | 6.26 ± 2.04 | 3.05 ± 1.02* |
| IL-10 | 22.54 ± 21.15 | 12.25 ± 23.11 | 13.24 ± 21.66 | 4.20 ± 10.28 |
| GM-CSF | 6.10 ± 8.49 | 23.48 ± 21.85 | 15.25 ± 14.76 | 1.13 ± 1.37 |
| IFNy | 18.36 ± 19.27 | 2.02 ± 3.34 | 0.0 ± 0.0 | 3.67 ± 8.98 |
| TNF-a | 3.52 ± 4.83 | 55.90 ± 34.78 | 11.67 ± 8.15 | 0.0 ± 0.0 |
| IL-1 a | 4.99 ± 0.58 | 8.13 ± 1.70* | 10.08 ± 1.29* | 5.72 ± 2.20 |
| IL-3 | 0.49 ± 0.67 | 2.42 ± 2.97 | 0.46 ± 0.56 | 0.20 ± 0.22 |
| IL-6 | 17.76 ± 16.41 | 175.24 ± 81.57 | 45.39 ± 27.75 | 38.53 ± 12.22 |
| IL-12(p40) | 3.41 ± 2.87 | 303.49 ± 167.51* | 87.15 ± 30.94* | 58.96 ± 41.83* |
| IL-12 (p70) | 6.64 ± 4.28 | 6.96 ± 6.67 | 3.19 ± 2.96 | 0.0 ± 0.0* |
| IL-17 | 2.96 ± 4.14 | 7.86 ± 9.09 | 3.36 ± 3.70 | 4.04 ± 4.44 |
| G-CSF | 8.18 ± 3.69 | 56.05 ± 25.24* | 25.15 ± 11.30* | 60.92 ± 21.38* |
| KC | 82.91 ± 33.59 | 514.41 ± 421.52 | 1174.97 ± 753.79* | 74.92 ± 33.58 |
| MIP-1 a | 68.34 ± 60.53 | 198.14 ± 57.05* | 163.78 ± 38.25* | 119.82 ± 64.02 |
| RANTES | 1.26 ± 2.82 | 202.72 ± 54.49* | 6.08 ± 7.07 | 259.67 ± 73.15* |
| TARC | 0.0 ± 0.0 | 23.33 ± 20.42* | 113.89 ± 39.19* | 0.65 ± 1.58 |

*p < .05 Dunnett's t-test of significance, compared to sham control ^{a} N = 5

Results for individual animals were reported as percent of the lymphocyte-gated population isolated by whole-lung digestion and density gradient centrifugation. The lymphocyte gate was selected based on forward and side scatter characteristics of the CD3-stained cells. The gate location was the same for lymphocytes in peripheral blood, spleen, and lung.

Staining of pulmonary lymphocytes with acceptable, distinct population separations and low background fluorescence was obtained for the following antibody combinations, as shown in Tables 9, 10, 11 and 12 below:

TABLE 9

| | | CD3-PECy7, CD4-PE, CD8-FITC (percent) | | | | |
|---|---|---|---|---|---|---|
| | CD3 | | CD4 | | | |
| LPS | CD8 F Cy7 | | PE | CD3/CD4 | CD3/CD8 | CD4/CD8 |
| 1001 | 12 | 26 | 13 | 12 | 11 | 0.5 |
| 1002 | 13 | 29 | 13 | 11 | 12 | 0.7 |
| 1003 | 14 | 32 | 15 | 14 | 13 | 0.2 |
| 1004 | 13 | 29 | 13 | 11 | 12 | 0.4 |
| Control 1 | 5 | 20 | 15 | 15 | 4.5 | 0.1 |
| Control 2 | 3.6 | 16 | 11 | 11 | 3.4 | 0.1 |
| Control 3 | 2.7 | 13 | 7.7 | 7.1 | 2.6 | 0.1 |
| Control 4 | 5.1 | 23 | 17 | 16 | 4.9 | 0.1 |

TABLE 10

| | CD69-PE, CD3-FITC, CD25-PECy7 (percent) | | | | |
|---|---|---|---|---|---|
| LPS | CD3 F | CD69PE | | CD3/CD69 | CD3/CD25 |
| 1001 | | | | | |
| 1002 | 30 | 30 | 6.9 | 13 | 3.8 |
| 1003 | 31 | 33 | 7.7 | 13 | 4.1 |
| 1004 | 29 | 32 | 7.7 | 14 | 4.6 |
| Control 1 | 21 | 1.9 | 3.6 | 0.9 | 0.7 |
| Control 2 | 16 | 1.8 | 2 | 0.7 | 0.3 |
| Control 3 | 9.7 | 1.6 | 4.5 | 0.8 | 0.6 |
| Control 4 | 21 | 2.1 | 2.9 | 0.9 | 0.6 |
| Stim lung | 14 | 1.8 | 4.6 | 1.9 | 2.6 |

Data for #1001 with this 3-color combination were lost due to incorrect population gating.

TABLE 11

| | | CD25-PECy7, CD8-FITC, CD69-PE (percent) | | | |
|---|---|---|---|---|---|
| LPS | CD8 F | CD69PE | CD25Cy7 | CD81CD69 | CD81CD25 |
| 1001 | 13 | 41 | 12 | 8.4 | 2 |
| 1002 | 12 | 29 | 9 | 5.5 | 0.4 |
| 1003 | 14 | 30 | 9.2 | 5.5 | 0.3 |
| 1004 | 14 | 29 | 8.8 | 5.8 | 0.3 |
| Control 1 | 4.6 | 1.5 | 6.5 | 0 | 0.1 |
| Control 2 | 4 | 1 | 4 | 0.1 | 0 |
| Control 3 | 2.1 | 1.2 | 7.8 | 0 | 0 |
| Control 4 | 5 | 1.4 | 4 | 0 | 0 |
| Stim lung | 4.8 | 1.3 | 5 | 0.6 | 0.6 |

TABLE 12

| | | CD4-PE, CD8-FITC, CD25-PECy7 (percent) | | | |
|---|---|---|---|---|---|
| | | CD25 | | | |
| LPS | CD8 F | Cy7 | CD4 PE | CD4/CD25 | CD8/CD25 |
| 1001 | 13 | 11 | 12 | 3.8 | 1.8 |
| 1002 | 13 | 4.7 | 14 | 1.6 | 0.3 |
| 1003 | 12 | 7.5 | 13 | 2.1 | 0.7 |
| 1004 | 14 | 5.1 | 13 | 1.6 | 0.5 |
| Control 1 | 5 | 3.2 | 15 | 0.5 | 0.1 |
| 2 | 4.1 | 2.6 | 13 | 0.4 | 0.1 |
| 3 | 2.4 | 4.7 | 7 | 0.3 | 0.1 |
| 4 | 5.5 | 2.5 | 20 | 0.5 | 0.2 |

Results of ConA stimulation of splenic, peripheral blood, and pooled pulmonary lymphocytes for positive controls were equivocal.

Results Histopathology

H&E-stained sections of lung tissue were examined from six mice in each of the following groups: sham control, LPS only, smoke only, and Smoke+LPS.

A cellular infiltrate consisting primarily of neutrophils with lesser numbers of macrophages was present in alveoli of all mice exposed to LPS only. This lesion was coded as: (1) suppurative inflammation of alveoli, and (2) PAM infiltrates in alveoli; both lesions were graded as minimal in all mice.

A mixed inflammatory cell infiltrate composed of neutrophils and macrophages was present in alveolar ducts of all mice exposed to smoke only, with a few macrophages in adjacent alveoli. FIG. 2 is a photograph that shows mixed inflammatory infiltrate.

This change was coded as: (1) alveoli, PAM infiltrate, and (2) alveoli and alveolar ducts, mixed inflammatory cell infiltrate. Both lesions were graded as minimal in all mice.

Mice exposed to both LPS and smoke had a diffuse mixed inflammatory cell infiltrate involving alveoli and alveolar ducts of all lung lobes.

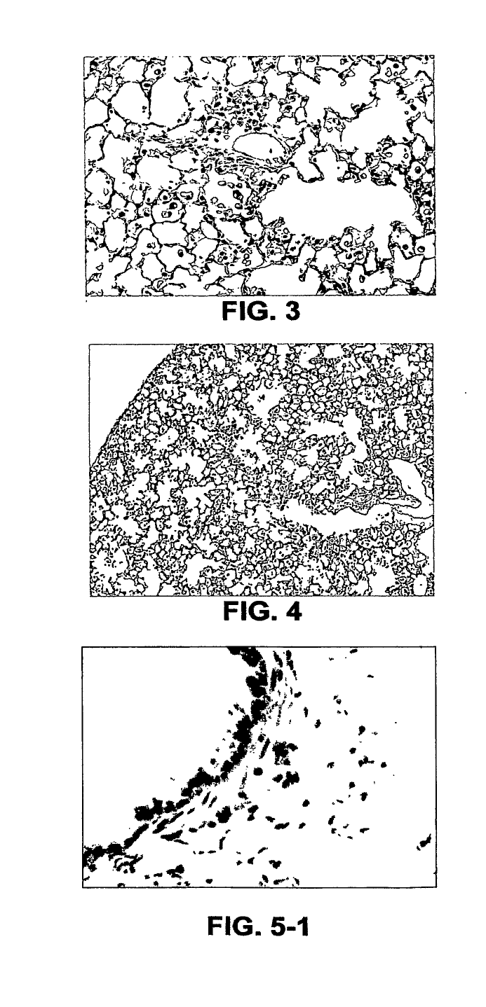

FIG. 3 shows diffuse mixed inflammatory cell infiltrate;

FIG. 4 shows the diffuse cellular infiltrate composed of predominately neutrophils in lung sections for the LPS group.

The infiltrate in alveoli was predominantly macrophages with slightly fewer neutrophils. This change was coded as: (1) alveoli, suppurative inflammation, minimal; (2) alveoli, PAM aggregates, mild; and (3), alveoli and alveolar ducts, mixed inflammatory infiltrate, mild.

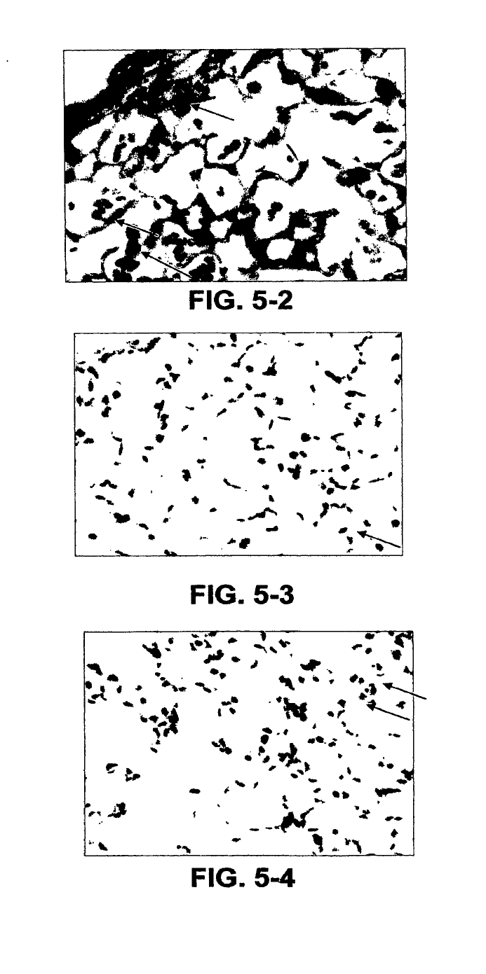

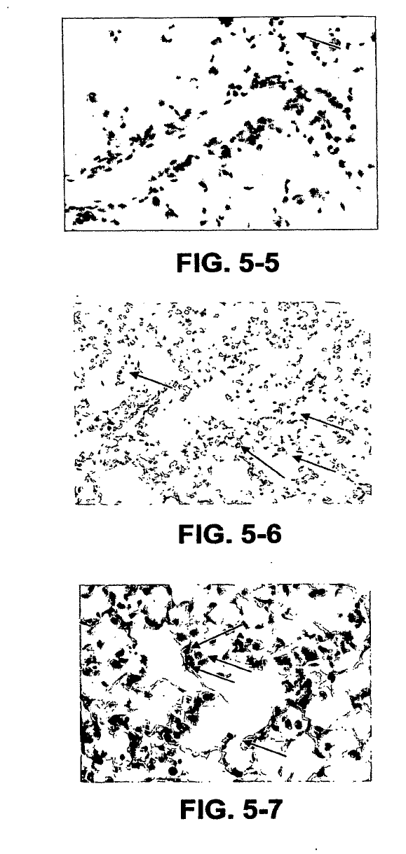

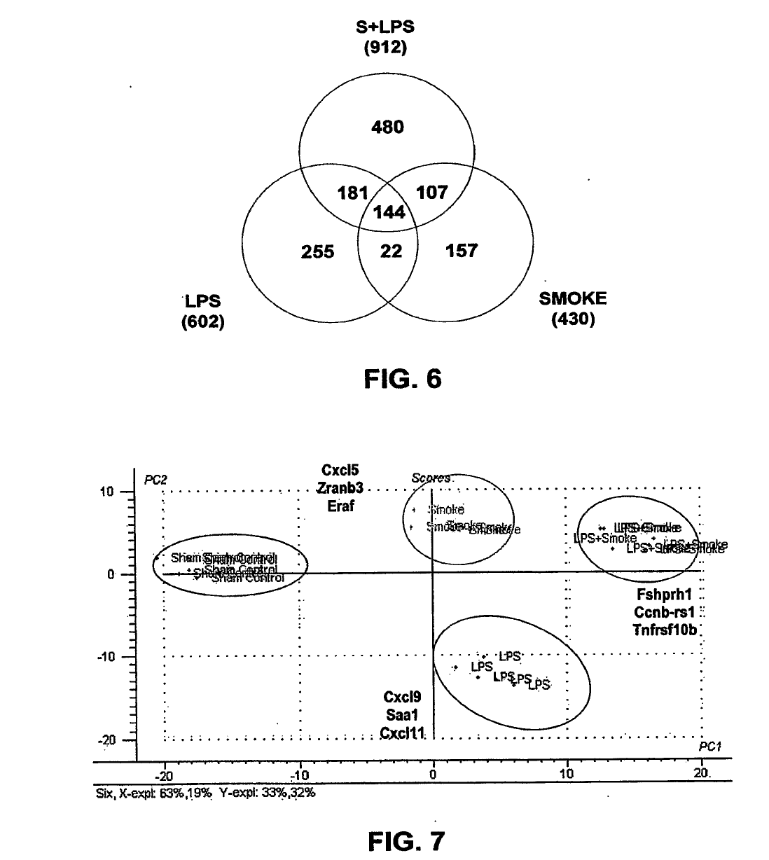

Histopathology results indicated augmented inflammatory responses for the Smoke+LPS exposure compared to LPS or Smoke exposure alone. The lung apoptosis demonstrated a similar difference between Smoke+LPS and either LPS or Smoke groups (See FIGS. 2, 3, 4 and also FIG. 5-6).

Apoptosis/TUNEL Assay

The controls had little to no apoptotic cells. The number of apoptotic cells was visually higher in the Smoke+LPS animals than in the smoke exposed animals and the number of the apoptotic cells was similar in the LPS exposed and Smoke exposed animals. No formal assessment was done on these animals. Photos of representative animals are in FIG. 5-1 through FIG. 5-7.

Counting of apoptotic cells was done using the following method: using a 40× objective and an ocular 10×10 grid, counting of labeled apoptotic alveolar cells was done starting from the second grid in from the edge of the lung and counting every other grid until five grids were counted.

The following TABLE 13 reflects the TUNEL Assay results:

TABLE 13
Apoptotic Cells (Mean ± SD; n = 6)

| Exposure Regimen | Labeled Cells |
|---|---|
| Control | 1.7 ± 0.8 |
| LPS | 5.3 ± 2.3 |
| Smoke | 5.0 ± 2.4 |
| Smoke/LPS | 17.2 ± 2.2 |

Clinical pathology findings for the inflammatory response in LPS exposed mice, as determined by BAL fluid analysis, was similar to the smoke-exposed mice. The BAL fluid from Smoke+LPS group also exhibited evidence of inflammation. Carboxyhemoglobin was elevated in both the smoke and Smoke+LPS groups confirming smoke exposure, yet carboxyhemglobin in the Smoke+LPS group was approximately ½ that of the smoke group. The difference for this is not clear; no clear difference in respiratory physiology was evident in the smoke vs Smoke+LPS groups. One possibility may be that the increased number of inflammatory cells in the lung may have decreased the amount of CO entering the bloodstream, either through direct uptake of CO themselves or indirectly via simple physical disruption of CO diffusion (e.g. thickening the alveolar interstitium and decreasing gas uptake).

However, potential differences in dosimetry alone do not explain the discrepancy that the Smoke+LPS group displayed significantly greater cellular infiltration but without a concomitant increase in enzymes and cytokines. While not wishing to be bound by theory, the inventors believe that co-exposure of Smoke+LPS did stimulate cellular infiltration into the lower respiratory tract (causing an increase in BAL leukocytes) above the level caused by Smoke alone, but the Smoke exposure suppressed or altered the activation of leukocytes (causing a decrease in BAL cytokines). This could be associated with immunosuppressive effects of acute cigarette smoke exposures which may lead to prolonged survival of infectious agents, contributing to reduced clearance and greater damage to the pulmonary architecture.

In general, the inflammatory process, as assessed by an increase in inflammatory cells (e.g. macrophages, neutrophils, lymphocytes) was greatest in the LPS and Smoke+LPS groups. The total cell, PAM, and PMN counts in smoke exposed animals were lower than LPS or Smoke+LPS groups. LPS exposed mice had the highest number of PMNs (LPS>Smoke+LPS=smoke>sham) while Smoke+LPS exposed animals had the greatest increase in PAMs (Smoke+LPS>LPS=smoke>sham).

Biochemical indicators of inflammation or cell damage were not consistently greater in one exposure group, although all exposure groups were greater than the control. The Smoke+LPS group had the greatest increase in LDH (Smoke+LPS>smoke>LPS>sham), which is an indicator of cellular degeneration and/or necrosis, while the smoke group had the greatest increase in NAG, an indicator of enzymatic release from leukocytes as well as an indicator cellular degeneration (smoke>Smoke+LPS>LPS>sham). Protein, which if increased suggests increased vascular permeability, was elevated to a similar degree in all exposed groups.

Elevations in cytokines/chemokines varied between groups. Only two cytokines, IL-12p40 and G-CSF were elevated in all groups, with LPS>smoke>Smoke+LPS for IL-12p40 and Smoke+LPS≈LPS>smoke for G-CSF. Bioactive IL-12 is a composite of p35 and p40 subunits. Expression of the p40 gene usually exceeds that of p35 resulting in p40 homodimers that antagonize bioactive IL-12p70 or resulting in p40/p19 (IL-23) heterodimers that, with IL-12, stimulate memory T helper type one cells (Th1). IL-12p40 is often markedly induced secondary to an inflammatory agent (i.e., LPS) while IL-12p35 production is constitutively expressed and/or less responsive to inflammatory stimuli. G-CSF plays a role in neutrophil development/maturation as well as serving as a chemotactic signal for neutrophil migration.

Significant increases in IL-1a, MIP-1a, and TARC were present in LPS and smoke groups with LPS smoke for IL-1a and MIP-1a and smoke>LPS for TARC. IL-1 (α or β) has many functions including inducing a fever, leukocytosis and activation of T lymphocytes. MIP-1a is a monocyte chemoattractant while TARC is a lymphocyte-directed CC chemokine which specifically chemoattracts type 2 CD4+ T cells.

RANTES, which costimulates T cell proliferation and IL-2 production in the context of anti-CD3 activation and is chemotactic for monocytes/macrophages and T cells, was significantly increased in LPS and Smoke+LPS groups with a similar magnitude increase in either group.

IL-1β, TNF-a, IL-6, were significantly elevated in the LPS exposed group alone, although TNF-a was slightly increased in the smoke group and IL-6 was slightly elevated in both the smoke and Smoke+LPS groups. All three of these cytokines play a role similar to that described for IL-1a.

Significant increases in KC were present only in Smoke exposed animals compared to the sham control; however KC was also elevated in LPS exposed animals (approximately % the mean value when compared to mice exposed to smoke alone). KC is a potent inducer of neutrophil activation and migration.

IL-2, IL-5, and IL-12p70 were significantly reduced in Smoke+LPS exposed animals only. IL-2 is produced upon antigenic stimulation of T cells and is vital to the cellular expansion required for a productive immune response. A lack of IL-2 production results in the development of an unresponsive state in Ag-stimulated T cells. IL-5 is required for eosinophil growth and differentiation (produced by Th2 cells). IL-12p70 is the active form of IL-12 and as described above includes the p40 and p35 subunits. Concentrations of IL-12p70 (and thus the activity of IL-12) may not correspond with IL12p40, which can either form homo or heterodimers with biological actions different than IL-12p70. A reduction in IL-12p70 may indicate decreased biological activity of IL-12 (even with an increase in IL-12p40) and less stimulation of a Th1 type response.

Microscopic examination of lung sections from mice exposed to LPS, smoke, or Smoke+LPS together indicated a predominantly macrophage response in alveolar ducts and adjacent alveoli to smoke only, and a mixed neutrophilic and macrophage response to LPS and smoke together. Only the lesions in mice exposed to Smoke+LPS were graded as mild, all others were minimal.

The Smoke, LPS, or combination of Smoke+LPS exposure regimens caused differential inflammatory responses in the lung of exposed mice without causing acute moribundity. Microscopic examination as well as BAL cytology consistently showed greater cellular infiltration for the Smoke+LPS group compared to LPS or Smoke alone (synergistic effects), with a cellular composition more closely resembling the Smoke group than the predominantly neutrophil response of LPS alone. At the same time however, the co-exposure of Smoke+LPS appeared to suppress the activation of recruited leukocytes, releasing less of BAL cytokines than groups exposed to Smoke or LPS alone (antagonistic).

The apparent difference in inflammatory profile (i.e., BALF cytology) may be limited to acute responses as in this study and change under chronic exposure condition (e.g., lymphocytic infiltration becomes dominating).

Also, lung transcriptomics and proteomics identified substantial quantity of genes and proteins unique or common among different groups.

### EXAMPLE II

### Gene Expression Profiling in Lung Tissues from Mice Exposed to Smoke, LPS, and Smoke+LPS by Inhalation

Animals and Exposure

AKR/J male mice were purchased from Jackson Laboratory (Bar Harbor, Me.). Groups of 6 mice were exposed via nose-only inhalation to one of the following: i) HEPA-filtered air (sham control group); ii) mainstream cigarette smoke at 250 μg/L wet total particular matter (WTPM) for 6 hrs/day, 5 days/week (smoke group); iii) 0.5 μg LPS/mouse for 1 hr/day, twice per week (LPS group); or iv) cigarette smoke at 250 gg/L WTPM, 5 hrs/day, 5 days/week, plus 0.5 gg LPS/mouse for 1 hr/day, twice per week after smoke exposure (Smoke+LPS group) for 3 consecutive weeks.

When not exposed to LPS, mice were exposed to HEPA-filtered air, so the exposure periods for each group were always 6 hrs per day. Cigarettes were 2R4F reference cigarettes procured from University of Kentucky (Lexington, Ky.). LPS from Escherichia coli serotype 055:B5 phenol extract was purchased from Sigma-Aldrich (Saint Louis, Mo., catalog number L2880). The same lot of LPS was used for the whole study. The Certificate of Analysis for the lot used showed an endotoxin level of 3,000,000 EU/mg. One extra mouse from the Smoke+LPS group was also processed for RNA isolation and microarray analysis.

Necropsy, Lung Tissue Collection, and RNA Isolation

Mice were sacrificed by IP injection of pentobarbital in the following morning of the last day of exposure. Right lung lobes from each group of mice were cut into small pieces and collected into RNAlater® (Qiagen, Valencia, Calif.) based on manufacture specified protocols. Total RNA was extracted from the lung tissues using RNeasy Midi Kits purchased from Qiagen (Valencia, Calif.). 0.1-0.2 g lung tissue for each mouse was added to 8 ml lysis buffer and homogenized for 30-60 seconds using an Omni International homogenizer with disposable generator probes (Marietta, Ga.). The homogenized tissue samples were frozen at approximately −70° C. and RNA was isolated following the kit instruction. RNA was further concentrated using YM-100 Microcon centrifuge filter devices (Billerica, Mass.). The ratio of A260/280 was measured using a spectrophotometer to evaluate the purity of RNA. Sample purity was also evaluated using an Agilent Bioanalyzer 2100 (Foster City, Calif.).

Microarray Data Acquisition and Preprocessing

Samples were prepared for hybridization to Affymetrix GeneChip microarrays using Affymetrix reagents and protocols. cDNA was synthesized from RNA using a one cycle cDNA synthesis kit. Biotinylated RNA was then synthesized from cDNA using an IVT labeling kit after incubating at 37° C. for 16 hours. The biotinylated RNA was fragmented and hybridized to an Affymetrix Mouse Genome 430 2.0 microarray for 16 hours at 45° C. in a hybridization oven rotating at 60 RPM. The microarray was washed and stained with streptavidin-phycoerythrin using an Affymetrix Fluidics Station 450. The array was then scanned using an Affymetrix GeneChip Scanner 3000. The chip data was analyzed using Affymetrix GeneChip Operating Software (GCOS) version 1.2.

Microarray Data Analysis

The microarray data was imported into Bioconductor for further analysis. The quality of each sample was first evaluated by visually inspecting the distribution of genes in graphs and by calculating sample similarity values as correlation coefficients. One sample from the LPS group and one from the Smoke+LPS group were determined to be outliers and removed from further analysis (2 out of total 25 samples).

Data were quantile normalized and subjected to one-way ANOVA parametric test using Benjamini and Hochberg False Discovery Rate for multiple testing correction for each treatment group compared to sham control. A P value of <0.05 was considered significant. Genes that changed 2-fold or greater in the treated groups compared to the sham control group were identified by filtering with fold change. See FIG. 6.

Differentially expressed genes were clustered by hierarchical clustering using GeneSpring™ (version 7.2, Redwood City, Calif.). The clustering was two-dimensional, i.e., for samples and for genes. The pattern of expression of changed genes was compared between groups. Biological process and functions of clusters were identified by querying against Gene Ontology (GO) term. The global relationships of individual samples were characterized by partial least squares discriminant analysis (PLSDA) using the genes that were changed in one or more treatment groups and displayed in a 3-D graph. See FIG. 7.

Differentially expressed gene lists were imported into Metacore™ (St. Joseph, Mich.) for functional and mechanistic analysis. A network for each treatment group was constructed by a strict “direct interaction” algorithm, which connects objects in the differentially expressed gene list by experimentally confirmed physical interactions. See FIGS. 8, 9 and 10. Genes were visualized on the network based on the up or down regulation in each treatment. Biological processes associated with changed genes in each group were generated by querying against the MetaCore™ database. The differences between changed gene lists were expressed as network connections by performing logical operations, i.e., subtracting one network from another network.

Results—Differentially Expressed Genes

The expression of 430 genes was changed in the Smoke group (increased or decreased at least 2 fold compared to sham control; p-value<0.05). Genes that were up- or down-regulated more than 10-fold in the Smoke group are listed in TABLE 14 below. The greatest increase was 20-fold (serum amyloid A1 and matrix metalloproteinase 12) and the greatest decrease was 24 fold (heat shock protein). These genes are believed to make especially good smoke exposure biomarkers.

TABLE 14
the Smoke Group

| SEQ ID No. | Probe_Set_Name | Gene Symbol | Fold Change |
|---|---|---|---|
| 1 | 450788_at | Saa1 | 20.30 |
| 2 | 1449153_at | Mmp12 | 20.11 |
| 23 | 1425151_a_at | Noxo1 | 18.77 |
| 24 | 1450826_a_at | Saa3 | 18.17 |
| 26 | 1425890_at | Ly6i | 17.34 |
| 121 | 1419728_at | Cxcl5 | 16.89 |
| 3 | 1434046_at | AA467197 | 14.40 |
| 48 | 1419209_at | Cxcl1 | 14.07 |
| 49 | 1419725_at | Slc26a4 | 12.76 |
| 50 | 1420589_at | Has3 | 12.61 |
| 51 | 1421792_s_at | Trem2 | 12.11 |
| 52 | 1422029_at | Ccl20 | 11.84 |
| 122 | 1455544_at | Zranb3 | 11.46 |
| 26 | 1447845_s_at | Vnn1 | 11.09 |
| 4 | 1428034_a_at | Tnfrsf9 | 10.79 |
| 27 | 1426464_at | Nr1d1 | 0.097 |
| 5 | 1452318_a_at | Hspa1a | 0.057 |
| 5 | 1427127_x_at | Hspa1a | 0.046 |
| 5 | 1427126_at | Hspa1a | 0.042 |

There were 912 genes differentially expressed in the Smoke+LPS group (increased or decreased at least 2-fold, p<0.05). Genes that were up- or down-regulated more than 10-fold are listed in TABLE 15 below. These genes are believed to be good indicators of disease severity and could provide biomarker signatures of drug efficacy or exposure toxicity.

TABLE 15
LPS + Smoke Group

| SEQ ID No. | Probe_Set_Name | Gene Symbol | Fold Change |
|---|---|---|---|
| 1 | 1450788_at | Saa1 | 178.3863 |
| 127 | 1418652_at | Cxcl9 | 52.05582 |
| 6 | 1458297_s_at | Marco | 50.09645 |
| 24 | 1450826_a_at | Saa3 | 38.95058 |
| 6 | 1449498_at | Marco | 27.25317 |
| 51 | 1421792_s_at | Trem2 | 24.11661 |
| 7 | 1420438_at | Orm2 | 20.04476 |
| 28 | 1451128_s_at | Kif22 | 16.72067 |
| 29 | 1434695_at | Dtl | 14.25335 |
| 124 | 1436723_at | Fshprh1 | 13.06821 |
| 30 | 1416558_at | Melk | 12.96264 |
| 0 | 1436530_at | — | 12.75555 |
| 108 | 1429095_at | 1700022C02Rik | 12.66846 |
| 31 | 1451054_at | Orm1 | 12.42615 |
| 8 | 1450652_at | Ctsk | 12.18682 |
| 26 | 1425890_at | Ly6i | 12.06233 |
| 32 | 1417445_at | Kntc2 | 11.93043 |
| 1 | 1419075_s_at | Saa1 | 11.1325 |
| 3 | 1434046_at | AA467197 | 11.11992 |
| 125 | 1416076_at | Ccnb1-rs1 /// Ccnbl /// | 10.77793 |
| | | LOC434175 /// Ccnb1- | |
| | | rs5 | |
| 33 | 1426157_a_at | Cd209b | 0.090371 |
| 34 | 1425993_a_at | Hsp110 | 0.084169 |
| 34 | 1423566_a_at | Hsp110 | 0.076104 |
| 34 | 1452318_a_at | Hspa1a | 0.057538 |
| 27 | 1426464_at | Nr1d1 | 0.051191 |
| 5 | 1427126_at | Hspa1a | 0.044791 |
| 5 | 1427127_x_at | Hspa1a | 0.04313 |

The greatest increase was 178-fold (serum amyloid A 1) and the greatest decrease was 23-fold (heat shock protein). There were significant overlaps between the Smoke+LPS group gene list and the Smoke group or LPS group gene lists as demonstrated by a Venn diagram (FIG. 6). Among the 255 differentially expressed genes in Smoke group, 198 (78%) were also seen in the Smoke+LPS group, while 278 genes (57%) of the LPS gene list also appeared in the Smoke+LPS group gene list.

Partial Least Squares Discriminant Analysis (PLSDA)

Using the genes that were differentially expressed in one or more treatment groups (FIG. 6), PLSDA on treatment conditions was performed for all samples. Sham control groups were clearly separated from three exposure groups (FIG. 7). FIG. 7 shows a statistical analysis of genes to distinguish the groups: Smoke: Cxcl5 [SEQ ID No. 121], Zranb3 [SEQ ID No. 122], Eraf [SEQ ID No. 123]; LPS: Cxcl9 [SEQ ID No. 127], Saa1 [SEQ ID No. 1], Cxcl11 [SEQ ID No. 128]; and Smoke+LPS: Fshprh1 [SEQ ID No. 124], Ccnb-rs1 [SEQ ID No. 125], Tnfrsf10b [SEQ ID No. 126].

The three exposure groups were clearly defined, although they were much closer to each other than to the sham control group. The distance between the Smoke group and the Smoke+LPS group was shorter than the distance between the LPS group to the Smoke+LPS group. The majority of variance was accounted for by two components. The first component accounted for 63% of variance and the second component accounted for 33% of variance.

Using the PLSDA scores, specific genes discriminate the treatment groups:

For the SMOKE group, as shown in FIG. 7:

Cxcl5: chemokine (C-X-C motif) ligand 5, involved in chemotaxis, inflammatory response, immune response, signal transduction, sensory perception, response to stimulus processes; it is an extracellular protein and is believed to make a good biomarker of exposure.

Zranb3: zinc finger, RAN-binding domain containing 3; involved in DNA repair, response to DNA damage stimulus, induction of apoptosis, and negative regulation of survival gene product activity. This is believed to be a marker of a specific type of DNA damage caused by smoke exposure.

Eraf: erythroid associated factor; involved hemoglobin binding, protein folding, erythrocyte differentiation, protein stabilization, hemoglobin metabolism, and hemopoiesis. Again, this is believed to be a good marker of specific damage caused by smoke exposure.

For the SMOKE+LPS group, as shown in FIG. 7:

Follicle stimulating hormone primary response gene 1 (Fshprh1), or centromere protein 1 (Cenp1), which is required for faithful chromosome segregation during cell division but otherwise poorly characterized and tissue-specific expression unknown;

Cyclin b1 related sequence (Ccnb1-rs1), which is believed to regulate progression through the cell cycle;

Tumor necrosis family receptor superfamily member 10b (Tnfrsf10b), which is significantly down-regulated in the SMOKE+LPS group, is required for FADD-mediated apoptosis.

While not wishing to be held to theory, it is believed that these are good markers of the combined SMOKE+LPS disease phenotype, which are unique from LPS-induced injury.

Multiple-Linear Regression Analysis

Multiple-Linear Regression analysis of histopathology data against significant gene changes was to identify genes/proteins that may correlate with pathology changes. We applied this method to microarray data, which selected for genes that positively correlated with increasing inflammation from Control<Smoke=LPS<SMOKE+LPS. Using Pearson correlation distance metrics, we identified 25 up-regulated and 25 down-regulated genes that met a tolerance level of 2% (see TABLE 16 and FIG. 10).

TABLE 16
Up and down-regulated genes that correlate
with degree of inflammation.

| SEQ ID | | | | |
|---|---|---|---|---|
| No. | Gene Symbol | LPS | Smoke | LPS + SMOKE |
| 35 | Lig1 | 1.570 | 1.532 | 2.387 |
| 36 | Cdca5 | 2.422 | 2.244 | 5.495 |
| 37 | Parvg | 1.569 | 1.658 | 2.574 |
| 9 | Bub1b | 2.809 | 2.731 | 6.474 |
| 115 | 2810417H13Rik | 2.760 | 2.714 | 6.356 |
| 38 | Ptgds2 | 1.749 | 1.813 | 2.899 |
| 39 | Asf1b | 1.881 | 1.748 | 3.629 |
| 40 | Mpeg1 | 1.594 | 1.571 | 2.581 |
| 114 | 5730507H05Rik | 2.268 | 2.496 | 5.672 |
| 108 | 1700022C02Rik | 2.580 | 2.628 | 5.940 |
| 41 | Kntc1 | 1.710 | 1.615 | 2.619 |
| 42 | Hist1h2ao | 2.254 | 2.193 | 4.948 |
| 42 | Mphosph1 | 1.518 | 1.464 | 2.176 |
| 44 | Adh6b | 1.494 | 1.529 | 2.505 |
| 10 | Cdc2a | 2.561 | 2.882 | 6.788 |
| 45 | Pbk | 2.091 | 2.195 | 4.247 |
| 11 | Ctsb | 1.474 | 1.534 | 2.138 |
| 113 | C330027C09Rik | 1.820 | 1.889 | 3.805 |
| 12 | Tyrobp | 1.478 | 1.438 | 2.221 |
| 13 | BC024561 | 1.460 | 1.504 | 2.253 |
| 46 | Asah1 | 1.421 | 1.488 | 2.140 |
| 47 | Pira2 | 1.608 | 1.579 | 2.584 |
| 112 | 2610318C08Rik | 1.378 | 1.396 | 2.053 |
| 14 | Top2a | 2.227 | 2.312 | 4.795 |
| 53 | Dlg7 | 2.091 | 2.071 | 4.457 |
| 54 | Gnmt | 0.492 | 0.402 | 0.204 |
| 15 | Flt1 | 0.641 | 0.660 | 0.480 |
| 16 | Ptgfr | 0.723 | 0.724 | 0.473 |
| 55 | Hist2h3c2 | 0.553 | 0.509 | 0.329 |
| 56 | Atf5 | 0.658 | 0.672 | 0.450 |
| 57 | Kras | 0.634 | 0.696 | 0.461 |
| 67 | 1435194_at | 0.688 | 0.631 | 0.436 |
| 58 | Hspa4 | 0.697 | 0.708 | 0.436 |
| 66 | 1448612_at | 0.472 | 0.426 | 0.229 |
| 59 | Myst3 | 0.604 | 0.660 | 0.376 |
| 17 | Agtrl1 | 0.535 | 0.609 | 0.297 |
| 60 | Trim63 | 0.665 | 0.671 | 0.445 |
| 65 | 1429510_at | 0.705 | 0.720 | 0.489 |
| 111 | E230025E14Rik | 0.621 | 0.597 | 0.413 |
| 61 | Cpn1 | 0.545 | 0.518 | 0.339 |
| 64 | 1438704_at | 0.632 | 0.634 | 0.375 |
| 110 | E230015L20Rik | 0.382 | 0.373 | 0.167 |
| 63 | 1439284_at | 0.662 | 0.700 | 0.473 |
| 18 | Sfn | 0.593 | 0.502 | 0.267 |
| 19 | Myh6 /// Myh7 | 0.502 | 0.483 | 0.232 |
| 20 | Plagl1 | 0.496 | 0.469 | 0.251 |
| 21 | Cacnb2 | 0.589 | 0.579 | 0.299 |
| 22 | Sox11 | 0.432 | 0.404 | 0.219 |
| 109 | A330102K23Rik | 0.436 | 0.447 | 0.134 |
| 62 | 1459552_at | 0.643 | 0.655 | 0.444 |

Many of them, such as Bub1b, Cdc2a, and Top2A, have been implicated in the development or progression of lung cancer. Ptgds2, also identified, has a role in late phase allergic reactions in the pathophysiology of bronchial asthma, and gamma-parvin (Parvg) along with integrin-linked kinase forms a complex that is involved in initial integrin signaling for leukocyte migration and leukocyte extravasation, an important step in the inflammatory response.

There were also several genes of unknown function that are identified herein that can also serve as unique biomarkers of lung inflammation.

Cluster Analysis

Hierarchical clustering for samples correctly separated the 23 samples into the 3 treatment groups and one sham control group based on the expression pattern of the 940 genes that were changed in one or more treatment groups (FIG. 11). Hierarchical clustering for genes generated a gene tree with three primary branches (A, B, and C) and many sub-branches at different levels. The overall patterns of gene expression in clusters A and C were similar while there were some differences in sub-clusters among the three treatment groups.

Cluster A was down-regulated and is related to heat shock response and chaperone activities.

Cluster C overall was up-regulated and is related to many biological processes or functions, including host-pathogen interaction, signal transduction, immune response, inflammatory response, etc.

In cluster B, several sub-clusters had clearly different expression patterns among the three treatment groups as demonstrated in the enlarged cluster B in the middle panel of FIG. 11, including sub-clusters D, E, F, G, and H.

Sub-cluster D genes were expressed at higher levels in the Smoke+LPS group than the Smoke and the LPS group. Sub-cluster D was further enlarged and individual genes in the cluster are displayed in FIG. 11.

Sub-cluster E genes were up-regulated in the LPS group compared to the Smoke and the Smoke+LPS groups.

Sub-cluster F genes were up-regulated in the LPS group compared to the Smoke and the Smoke+LPS group.

Sub-cluster G genes were up-regulated in the Smoke and the Smoke+LPS groups compared to the LPS group.

Sub-cluster H genes were up-regulated in the Smoke+LPS group compared to the Smoke and the LPS groups.

The primary function of these genes is related to microtubular dynamics. No definitive function was identified for Sub-clusters E to H by querying the GO database.

Network Analysis

Smoke

One single network was generated using the direct interaction algorithm for the changed genes in the Smoke group. This integrated network included several core modules (see FIG. 8).

FIG. 8 shows the gene networks after applying the direct interactions algorithm to the list of differentially expressed genes in the Smoke group. One single network was generated. Colored symbols represent genes (network nodes). Red solid circles represent up-regulated genes and blue solid circles represent down-regulated genes. Primary function modules are highlighted with green circles. Primary function modules include heat shock response (HSP70, HSP90, etc), mitotic process (CDK1 and Cyclin A, etc.), and DNA damage check point (Nucloesome, Granzyme A, etc.).

One module was related to the acute response process, molecular chaperone, and protein folding, with HSP90 and HSP70 gene families as central roots. Genes of this module were down-regulated, indicating compromised protective capability of the organism against injury and stress.

A second major module was the up-regulated mitotic process with CDK1 and Cyclin A as central roots. The module for DNA damage check point and double strand break repair was down-regulated (with nucleosome related gene group and granzyme A as the central roots). Biological processes associated with the differentially expressed genes in the Smoke group were identified by querying the MetaCore™ database. The top ranked biological processes are listed in TABLE 17, including inflammatory response, neutrophil chemotaxis, response to heat, immune response, response to unfolded proteins, and the like.

TABLE 17
Biological processes in the Smoke group

| PROCESS | NO. of genes | P-VALUE |
|---|---|---|
| inflammatory response | 22 | 1.33E−09 |
| neutrophil chemotaxis | 9 | 2.21E−09 |
| response to heat | 8 | 2.63E−09 |
| chemotaxis | 15 | 3.58E−09 |
| immune response | 26 | 9.31E−08 |
| sensory perception | 16 | 1.33E−06 |
| response to unfolded protein | 8 | 1.93E−06 |
| mitosis | 12 | 2.38E−06 |
| G-protein signaling, coupled to cyclic | 8 | 5.36E−06 |
| nucleotide second messenger | | |
| anti-apoptosis | 13 | 8.40E−06 |
| cell-cell signaling | 19 | 1.02E−05 |
| signal transduction | 46 | 5.44E−05 |
| antimicrobial humoral response (sensu | 8 | 1.67E−04 |
| Vertebrata) | | |
| cell cycle | 18 | 3.34E−04 |
| cellular defense response | 8 | 4.47E−04 |
| G-protein coupled receptor protein | 19 | 6.44E−04 |
| signaling pathway | | |
| positive regulation of cell proliferation | 11 | 1.23E−03 |
| negative regulation of cell proliferation | 11 | 1.28E−03 |
| cell surface receptor linked signal | 12 | 1.39E−03 |
| transduction | | |
| cell motility | 9 | 1.43E−03 |
| cell adhesion | 18 | 2.61E−03 |
| organ morphogenesis | 11 | 2.93E−03 |
| protein folding | 9 | 3.14E−03 |
| regulation of progression through cell | 14 | 5.15E−03 |
| cycle | | |
| DNA repair | 9 | 7.02E−03 |
| protein amino acid phosphorylation | 17 | 7.67E−03 |

LPS

FIG. 9 shows the gene networks after applying the direct interactions algorithm to the list of differentially expressed genes in the LPS group. Four networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

Smoke+LPS

Five networks were generated using the direct interaction algorithm for the changed genes in the Smoke+LPS group (see FIG. 12). FIG. 12 shows the gene networks after applying the direct interactions algorithm to the list of differentially expressed genes in the Smoke+LPS group. One large network and four smaller networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

Most of the core modules seen in these networks, including the G-protein coupled receptor protein signaling pathway module, the molecular chaperone and the acute response modules, DNA cell cycle regulation and mitosis, were also observed in the networks generated from changed genes in the Smoke or LPS treatment group.

Additionally, one network/module related to cell cycle regulation (cycle E as central root), one network/module related muscle contraction (with Actin and TNNT2 as central roots), and one function module related to NADPH oxidase activity (gp91-phox, p22-phox, and p47-phox) were also observed.

The biological processes associated with the differentially expressed genes in the Smoke+LPS group included several of the major biological processes associated with the changed genes in the Smoke groups but the order of the processes were unique to this combined treatment. The three top scoring processes described a dramatic induction in mitosis, cell division and cell cycle control (See TABLE 18 below).

TABLE 18
Biological processes in the Smoke + LPS group

| PROCESS | No. of genes | P-VALUE |
|---|---|---|
| mitosis | 34 | 9.30E−20 |
| cell division | 35 | 5.51E−17 |
| cell cycle | 55 | 3.04E−16 |
| inflammatory response | 39 | 3.33E−13 |
| neutrophil chemotaxis | 14 | 2.39E−12 |
| immune response | 50 | 1.11E−11 |
| response to unfolded protein | 16 | 1.90E−11 |
| response to heat | 11 | 1.71E−10 |
| phosphoinositide-mediated | 10 | 1.60E−08 |
| signaling | | |
| chemotaxis | 20 | 3.99E−08 |
| cellular defense response | 18 | 6.43E−08 |
| muscle contraction | 19 | 1.55E−07 |
| regulation of progression through | 36 | 2.07E−07 |
| cell cycle | | |
| complement activation | 10 | 4.06E−07 |
| positive regulation of cell | 25 | 5.28E−07 |
| proliferation | | |
| DNA replication | 18 | 7.52E−07 |
| elevation of cytosolic calcium ion | 13 | 3.29E−06 |
| concentration | | |
| circulation | 13 | 4.49E−06 |
| cytoskeleton organization and | 17 | 7.32E−06 |
| biogenesis | | |
| cell-cell signaling | 31 | 9.24E−06 |
| cell motility | 18 | 2.10E−05 |
| protein folding | 19 | 2.99E−05 |
| defense response | 17 | 4.09E−05 |
| DNA repair | 20 | 5.32E−05 |
| signal transduction | 85 | 5.37E−05 |
| antimicrobial humoral response | 12 | 1.48E−04 |
| (sensu Vertebrata) | | |
| cell adhesion | 36 | 1.56E−04 |
| calcium ion homeostasis | 10 | 1.86E−04 |
| lipid metabolism | 18 | 4.61E−04 |
| cell-matrix adhesion | 11 | 7.83E−04 |
| intracellular signaling cascade | 27 | 1.13E−03 |

To investigate whether there are any biological processes or networks/modules that are unique to the Smoke+LPS group, logical operations were performed to subtract the Smoke network and the LPS network from the Smoke+LPS network. Three small networks were identified (see FIG. 13), two of them with APC/CDC20 complex plus Cyclin B, and Aurora or the LPS network. FIG. 13 shows the subtraction of the Smoke network and the LPS network from the Smoke+LPS network by logical operation. Three small networks were generated. Colored symbols represent genes (network nodes). Red solid circles represent up-regulated genes and blue solid circles represent down-regulated genes. Function modules related to inflammatory response are highlighted with encircling red circles and other primary function modules are highlighted with encircling green circles.

One network/module that was truly unique to the Smoke+LPS group is related to muscle development or muscle contraction. Biological processes associated with changed genes observed only in the Smoke+LPS group include muscle contraction, calcium ion homeostasis, and lipid metabolism.

Gene expression or transcriptomics changes usually occur in early stage of disease development and may be used to predict disease outcome and shed light on mechanisms of disease development.

The number of differentially expressed genes in the Smoke+LPS group (912 genes) is significantly greater than those in the Smoke group (430 genes) and LPS group (602 genes), indicating a greater response in the combined treatment group in terms of changes of gene profiles (see FIG. 6).

The consistency of changed gene profiles between the Smoke group and the Smoke+LPS group was reflected by the fact that 58% of changed genes in the Smoke group were also observed in the Smoke+LPS group. A similar percentage of differentially expressed genes in the LPS group (53%) were seen in the changed genes of the Smoke+LPS group. This indicates that the gene profile changes associated with Smoke or LPS exposure were significantly modified by the combined exposure regimen, both a suppressive immune response to LPS and an exacerbated inflammatory response to Smoke.

To further investigate the functional mechanism of the differentially expressed genes in each treatment group, a “signature networks” approach using the MetaCore™ software (Nokolsky et al., 2005) was employed. Networks composed of functional modules were constructed by connecting changed genes with experimentally confirmed physical interactions. After the initial steps of identifying differentially expressed genes and cluster analysis of these genes, the changes of networks/functional modules were identified to provide additional evidence of changed biological processes of a chemical or biological challenge.

One primary module in the network generated from changed genes in the Smoke group was related to acute response and chaperone activities with the heat shock genes HSP90, HSP70, HSP60, Hdj2 as the central roots. Heat shock proteins primarily protect cells by folding denatured proteins, stabilizing macromolecules, and targeting irreversibly denatured proteins for clearance. Reduced expression of heat shock proteins comprises the protective functions of organisms from injuries caused by heat, ischemia, hypoxia, free radicals, oxidants, etc. Under-expression of heat shock proteins may also affect immune and acute inflammatory responses against pathogens. Notably, although inflammatory response was identified as a biological process associated with changed genes, no primary function module for inflammatory response was present in the network, indicating that the number of up-regulated genes related to inflammation was relatively few compared to the other treatment groups.

Immune and inflammatory responses were the dominant components in the networks generated from changed genes in the LPS group, involving 3 primary function modules, as shown in FIG. 9. The heat shock response and molecular chaperone genes formed a much smaller function module, indicating that the down-regulation of heat shock response was insignificant compared to the up-regulation of inflammatory response in the LPS group.

All the function modules of the network generated from differentially expressed genes in the Smoke group were present in the networks of the Smoke+LPS group with varying degrees of expansion in the number of genes involved, as shown in FIG. 12. Function modules observed in the networks of the LPS group were also reflected in the networks of the Smoke+LPS group, but with more modifications. The number of genes and function modules related to inflammatory and immune responses were reduced in the Smoke+LPS group compared to the LPS group. The two function modules related to inflammation in the LPS networks were not observed in the Smoke+LPS group networks, and the ratios of genes in the changed gene list versus the genes in the category for the inflammatory response were different in the two groups (22, 43, and 39, for Smoke, LPS, and the Smoke+LPS groups, respectively, TABLES 17 and 18). P values for the inflammatory response in each group also indicate that the significance of inflammatory response in each group was in the order of LPS>Smoke+LPS>Smoke. On the other hand, there were significant overlaps in inflammatory genes involved among the treatment groups.

The reduced number of genes and function modules associated with inflammatory response in the Smoke+LPS group also can be partially attributed to suppression of smoke exposure on acute immune and inflammatory response. Smoking-induced COPD is characterized by increased levels of mixed inflammatory cell infiltration. However, this is the result of long term smoke exposure and represents a disease status, and may not be the initiating event. For example, acute smoke exposure actually decreased airway neutrophils in healthy intermittent smokers during the initial stage of smoking. The suppressive effect of cigarette smoke on acute inflammation is associated with down-regulation of neutrophil mobilizing cytokines. The production of IL-8 and granulocyte-macrophage colony-stimulating factor (GM-CSF) is critical for neutrophil recruitment and inhibition of these cytokines results in reduced influx of neutrophils. Cigarette smoke condensate inhibited LPS-induced GM-CSF and IL-8 production in human bronchial epithelial cells.

Smoke exposure-related suppression of acute immune and inflammatory response may represent another mechanism of COPD development in smokers. Acute inflammation may release enzymes that damage normal tissues. However, acute inflammation also has many beneficial effects, including destruction of invading microorganisms. Chronic inhalation of cigarette smoke may promote colonization of bacteria in the airways, resulting in chronic lung inflammation; thus, indirectly contributing to COPD development in smokers.

A small network/function module consisting of three genes (gp91-phox, p22-phox, p47phox) was observed only in the Smoke+LPS group. Gp91-phox and p22-phox are two subunits forming the core heterodimer of NAD(P)H oxidase, and p47-phox is a regulatory subunit of NDD(P)H oxidase. Imbalance of oxidants/antioxidants is believed to play an important role in pathogenesis of COPD. NAD(P)H is one of the major oxidant generating enzymes present in lung and is induced during inflammatory status. Gp91-phox and p47-phox (not p22-phox) were also up-regulated in the LPS group at a slightly lower level, but not in the Smoke group. Co-exposure to smoke and LPS may have enhanced the production of reactive oxygen species in the lung.

Function modules/networks that were exclusive to the Smoke+LPS mice were obtained by subtracting networks generated from the Smoke group or the LPS group from the networks of Smoke+LPS group. Two of the small networks generated, one with APC/CDC20 complex as the central root and one with nucleosome as the central root, were associated with mitosis and cell cycle regulation. Small individual networks/function modules related to mitosis and cell cycle regulation were seen in the Smoke and LPS groups as well. With more genes changed in the Smoke+LPS group, individual networks were connected and expanded to form a grand network.

The other function module was related to muscle development/contraction with TNNI3 (cardiac troponin I), INNT2 (cardiac troponin T), and Actin as central roots. Weakness of peripheral muscle and inspiratory muscle were reported to occur in COPD patients, and it was concluded that the reduced muscle contractility is related to COPD. The down-regulated muscle contraction/development gene expression found in the initiating stage of COPD development described herein shows that dysfunction of respiratory tract muscle may be also a cause of smoke-induced COPD.

Also, inspection of individual genes in the differentially expressed gene lists revealed novel markers for monitoring COPD initiation and progression. The most up-regulated genes in the Smoke group were Serum amyloid A1 and MMP12.

MMP12 is increased in the smoke and Smoke+LPS groups. MMP12 is a matrix metalloproteinase that preferentially degrades elastin and has been implicated in COPD development. MMP12 knockout mice did not develop emphysema following smoke exposure compared to smoke-exposed normal mice, indicating MMP12 is critical in smoke-induced lung injury.

NOXO1 (up-regulated in Smoke group and in the Smoke+LPS group) is an organizer protein that activates NADPH oxidase (NOX1). NADPH Oxidase is a major source of reactive oxygen species and oxidative stress is considered to play an important role in the pathogenesis of COPD. Thus, increased NOXO1 expression is a novel marker for COPD development through imbalance of oxidant/antioxidants.

The Serum amyloid A gene group (including Saa1, Saa2, and Saa3) is up-regulated in all three groups) and is an acute phase systemic inflammation marker that can be induced by LPS treatment. The average fold-increase of Saa in Smoke+LPS group was lower than that in the LPS group, consistent with the observation that acute inflammation response in Smoke+LPS mice was attenuated by smoke exposure in the Smoke+LPS group mice.

MARCO (increased in the Smoke+LPS group) mRNA was one of the most up-regulated genes in splenic dendritic cells following LPS activation and in GM-CSF-treated microglial cells. MARCO is a scavenger receptor expressed in macrophages and is believed by the inventors herein to play a significant role in LPS-induced inflammation response.

Yet another aspect, there is provided herein a method for the analysis of differentially expressed genes in each treatment group using several different approaches. Hierarchical clustering showed that while the overall pattern of differentially expressed genes in all three treatment groups was similar, there were quantitative and qualitative differences in sub-clusters. Signature network construction identified biological processes and function modules involved in changed genes of each treatment group. Decreased heat shock response and chaperone activity, increased immune and inflammatory response and increased mitosis were the shared changes in all three groups, but the number of genes in corresponding clusters or function modules/networks varied among treatment groups.

One notable finding was the reduced number of genes and function modules/networks associated with inflammation in the Smoke+LPS group compared to the LPS group. This reduction was attributed to the decrease in inhaled smoke and LPS in the Smoke+LPS group mice and immune and inflammation-suppressive effects of acute smoke exposure.

In certain embodiments, it is possible to modify the current exposure regimen so smoke and LPS do not interfere with each other's intake. Co-exposure to smoke and LPS increased expression of genes related to muscle contraction and reactive oxygen species production, which have been reported relevant to COPD pathogenesis; however, LPS exposure did not potentiate the induction of genes related to inflammatory response by cigarette smoke exposure in mice.

### EXAMPLE III

### Marker Capability Development Related to an Animal Model for Chronic Obstructive Pulmonary Disease (COPD) Investigation Using LPS and Cigarette Smoke Exposure

Characterization of Lung Proteome IN Mice Upon LPS and/or Cigarette Smoke Exposure

This example combines mass spectrometry (MS) proteomic methodology with mixed effects linear statistical modeling to identify proteins whose concentrations change due to treatment effects. Lungs from mice exposed vis 3-week inhalation to LPS, cigarette smoke (Smoke) and Smoke+LPS or sham controls were digested with trypsin and evaluated by tandem mass spectrometry and FTICR (Fournier Transformed Ion Cyclotron Resonance)-MS approaches as described previously. SEQUEST® analysis of the MS data identified 3219 peptides corresponding to 2834 proteins on the NCBI database. Protein Prophet® reduced the number of identified proteins to 1240 by grouping isoforms and other database entries with highly similar amino acid sequences.

The variability in MS abundance (peptide peak area) due to treatment and processing was quantified with a mixed effects model fit with restricted maximum likelihood estimation. The resulting estimates of protein concentrations (treatment group relative to controls) revealed 383 up- and down-regulated proteins with a false discovery rate of 5%. For a protein to be considered to be either up or down regulated it had to be observed in 3 of 5 mice in a treatment group and have an abundance ratio of <0.67 or >1.5.

Using these criteria, 171 proteins were regulated by smoke exposure (133 were up-regulated and 39 were down-regulated), 119 proteins were regulated by LPS exposure (102 up-regulated and 17 down-regulated), and 179 proteins were regulated in the Smoke+LPS group (134 up-regulated and 45 down-regulated).

There were 10 proteins that changed more than 3-fold with Smoke exposure compared to the sham control group (see TABLE 19). The greatest protein changes observed were surfactant protein D (increased 10-fold) and haptoglobin (decreased 6-fold). These proteins are believed to provide good biomarker signatures of tissue response and damage to cell structure from smoke exposure.

TABLE 19

| | | | Example | Smoke |
|---|---|---|---|---|
| SEQ ID | | | Peptide | Fold |
| No. | REF_SEQ ID | Protein Description | Identification | Change |
| 89 | NP_033186 | surfactant associated | SATENAAIQQ | 10.2 |
| | | protein D | LITAHNK | |
| 80 | XP_915382 | PREDICTED: similar to | GITWGEDTLM | 3.7 |
| | | Cytochrome c, somatic | EYLENPKK | |
| 81 | XP_925763 | PREDICTED: similar to | NKGDSAYNTR | 3.64 |
| | | Integrin alpha-1 precursor | | |
| | | (Laminin and collagen | | |
| | | receptor) (VLA-1) | | |
| | | (CD49a) isoform 3 | | |
| 82 | NP_542126 | SH3 domain binding | IQYQLVDISQ | 3.57 |
| | | glutamic acid-rich | DNALRDEMR | |
| | | protein-like 3 | | |
| 83 | XP_485004 | PREDICTED: similar to | GFGFITFTNPE | 3.4 |
| | | Putative RNA-binding | HASDAMR | |
| | | protein 3 (RNA-binding | | |
| | | motif protein 3) | | |
| 84 | NP_032016 | Finkel-Biskis-Reilly | FVNVVPTFGK | 3.35 |
| | | murine sarcoma virus | | |
| | | (FBR-MuSV) | | |
| | | ubiquitously expressed | | |
| | | (fox derived) | | |
| 85 | XP_926145 | PREDICTED: similar to | EYQDLLNVK | 3.25 |
| | | neurofilament, heavy | | |
| | | polypeptide isoform 4 | | |
| 86 | NP_031457 | alkaline phosphatase 2, | DIDVIMGGGR | 3.15 |
| | | liver | | |
| 87 | NP_031789 | cysteine-rich protein 1 | TLTSGGHAEH | 3.1 |
| | | (intestinal) | EGK | |
| 88 | NP_059066 | haptoglobin | GSFPWQAK | 0.158 |

There were 12 proteins that changed more than 3-fold for Smoke+LPS as compared to sham control group (see TABLE 20). The greatest protein changes observed in the combined treatment group were again surfactant protein D (increased 6.7-fold) and haptoglobin (decreased 3.8 fold). These proteins are believed to provide good biomarker signatures of tissue response and damage to cell structure from combined smoke+LPS exposure.

TABLE 20

| | | | Example | |
|---|---|---|---|---|
| SEQ ID | | | Peptide | Smoke + |
| No. | REF_SEQ ID | Protein Description | Identification | LPS |
| 89 | NP_033186 | surfactant associated | SATENAAIQQL | 6.7 |
| | | protein D | ITAHNK | |
| 90 | NP_058089 | RNA binding motif | LFVGGLNFNT | 4.61 |
| | | protein 3 | DEQALEDHFSS | |
| | | | FGPISEVVVVK | |
| 83 | XP_485004 | PREDICTED: similar to | GFGFITFTNPE | 4.51 |
| | | Putative RNA-binding | HASDAMR | |
| | | protein 3 (RNA-binding | | |
| | | motif protein 3) | | |
| 91 | NP_062264 | lymphocyte specific 1 | LQQYTQATESS | 4.03 |
| | | | GR | |
| 92 | NP_032251 | hematopoietic cell | SAVGFNEMEA | 3.79 |
| | | specific Lyn substrate 1 | PTTAYK | |
| 82 | NP_542126 | SH3 domain binding | IQYQLVDISQD | 3.7 |
| | | glutamic acid-rich | NALRDEMR | |
| | | protein-like 3 | | |
| 84 | NP_032016 | Finkel-Biskis-Reilly | FVNVVPTFGK | 3.51 |
| | | murine sarcoma virus | | |
| | | (FBR-MuSV) | | |
| | | ubiquitously expressed | | |
| | | (fox derived) | | |
| 93 | XP_920646 | PREDICTED: similar to | SNSSEASSGDF | 3.37 |
| | | hematological and | LDLK | |
| | | neurological expressed | | |
| | | sequence 1 | | |
| 94 | NP_034578 | heterogeneous nuclear | EVYQQQQYGS | 3.29 |
| | | ribonucleoprotein A/B | GGR | |
| 87 | NP_031789 | cysteine-rich protein 1 | TLTSGGHAEH | 3.15 |
| | | (intestinal) | EGK | |
| 95 | NP_058020 | S100 calcium binding | TEFLSFMNTEL | 3.1 |
| | | protein A11 (calizzarin) | AAFTK | |
| 88 | NP_059066 | haptoglobin | GSFPWQAK | 0.264 |

The overlap between treatment groups is illustrated in FIG. 14 and indicates that there is a high degree of consistency between the treatment groups. Interestingly, almost half of the protein changes were common to all three treatment groups. For example, 89 of the proteins identified for the LPS group were also present in the Smoke and Smoke+LPS groups. Small numbers of proteins were unique to the LPS group; however, 37 proteins in the Smoke+LPS group were only present in that group and 42 proteins were common to smoke exposure groups but not regulated with LPS exposure alone.

Partial Least Squares Discriminant Analysis (PLSDA) Using the peptide data prior to roll-up into protein groups, PLSDA on treatment conditions was performed for all samples on all peptides that contained a complete dataset; that is, had quantitative measurements in all runs for all animals. Sham control groups were clearly separated from three exposure groups on principal component 1, and treatment groups were also clearly separated by principal component 3 (see FIG. 15).

Using the PLSDA scores, specific proteins can be identified which discriminate the treatment groups and are believed to perform as biomarker signatures of response to smoke exposure and the SMOKE+LPS disease phenotype:

For the SMOKE group:

Serine (or cysteine) peptidase inhibitor, clade A, member 1D (Serpina 1), also up-regulated in microarray data; involved in acute-phase response, defense response and immune response; this protein is in the same family as the alpha-1-antitrypsin proteins that are believed to be associated with COPD in smokers and non-smokers.

Cyclin fold protein 1 (CyclinN) belongs to the cyclin family, involved in phosphorylation and regulation of cell cycle; is a novel protein identified with alternatively splice exons in melanocytes and melanomas.

Procollagen, type i, alpha 1 (Fibrillar collagen) is a structural molecule, an extracellular matrix structural constituent conferring tensile strength; mutations in this gene are associated with idiopathic osteoporosis and a rare type of skin cancer called dermatofibrosarcoma.

For the SMOKE+LPS group:

Vimentin, a cytoskeletal component of intermediate filaments; apoptotic neutrophils express vimentin on their surface; these cells may participate in the development of autoantibodies directed against cytoskeletal proteins, a condition frequently reported in several inflammatory diseases.

AHNAK nucleoprotein isoform 1, involved in protein binding and nervous system development, Ahnak has a critical role in cardiac calcium channel function and its beta-adrenergic regulation; the carboxyl-terminal domain of Ahnak exerts a stabilizing effect on muscle contractility via its interaction with actin of thin filaments, providing a link between cardiac L-type Ca2+ channels and the actin-based cytoskeleton.

Periaxin isoform L, involved in axon ensheathment and mechanosensory behavior, this protein plays a significant role in the myelination of peripheral nerves. Nothing is known of its expression or regulation in lung tissue.

Multiple-Linear Regression Analysis

Multiple-Linear Regression analysis of histopathology data against significant protein changes was to identify proteins that may correlate with pathology changes. We applied this method to proteomic data, which selected for proteins that positively correlated with increasing inflammation from Control<Smoke=LPS<SMOKE+LPS. Using Pearson correlation distance metrics, we identified 11 up-regulated and 11 down-regulated proteins that met a tolerance level of 5% (see TABLE 21 and FIG. 16).

TABLE 21
Proteins that positively correlate with inflammatory profile from
histopathology data.

| SEQ | | | Fold Change | | |
|---|---|---|---|---|---|
| ID No. | Ref Seq ID | PROTEIN | LPS | SMOKE | LPS + SMOKE |
| 68 | NP_035010 | NCL | 1.35 | 1.23 | 1.62 |
| 96 | NP_031617 | CALR | 1.41 | 1.38 | 1.70 |
| 97 | XP_911341 | Chmp4b | 1.35 | 1.35 | 1.62 |
| 69 | NP_056549 | COL5A1 | 2.00 | 2.00 | 6.03 |
| 98 | NP_082751 | Ubap21 | 1.55 | 1.62 | 2.09 |
| 99 | NP_032933 | Ppia | 1.20 | 1.15 | 1.45 |
| 70 | NP_031713 | CFL1 | 1.15 | 1.17 | 1.41 |
| 100 | NP_291039 | WBSCR1 | 1.58 | 1.62 | 2.14 |
| 71 | NP_058540 | RPS28 | 1.55 | 1.51 | 2.09 |
| 72 | NP_035319 | PSME1 | 1.29 | 1.26 | 1.58 |
| 73 | NP_080296 | RPLP2 | 1.55 | 1.62 | 2.09 |
| 74 | NP_034085 | DPYSL2 | 0.79 | 0.87 | 0.69 |
| 75 | NP_033786 | ALDH2 | 0.62 | 0.58 | 0.47 |
| 101 | NP_034488 | GSTM1 | 0.68 | 0.69 | 0.52 |
| 76 | NP_033286 | SPNB2 | 0.93 | 0.95 | 0.87 |
| 102 | XP_918428 | Fusip1 | 0.78 | 0.69 | 0.62 |
| 77 | NP_038961 | VAPA | 0.76 | 0.55 | 0.38 |
| 103 | NP_038495 | ALDH1A1 | 0.72 | 0.72 | 0.55 |
| 78 | NP_033033 | RAC1 | 0.83 | 0.71 | 0.58 |
| 104 | NP_031479 | PRDX6 | 0.85 | 0.85 | 0.74 |
| 129 | XP_920894 | LOC639389 | 0.89 | 0.87 | 0.72 |
| 79 | NP_031611 | ANXA2 | 0.85 | 0.81 | 0.71 |

These include:

Nucleolin (NCL), which upon heat shock is relocalized from the nucleolus to the nucleoplasm in a p53-dependent fashion, whereupon it binds replication protein A and inhibits DNA replication initiation;

Calreticulin (CALR), found to be up-regulated in rat bronchoalveolar lavage fluid proteins associated with oil mist exposure; act as a receptor for surfactant proteins SP-A and SP-D, which are involved in surfactant homeostasis and pulmonary immunity; and

Procollagen, type V, alpha 1 (Col5SA1), immunity to type V collagen, released by and collagen V-reactive lymphocytes express autoimmune cytokines IL-17 and IL-23, associated with lung disease.

Biological Processes (and/or Effects)

To relate proteins with significant abundance changes to specific biological effects in exposed animals, METACORE software (GeneGo, Inc.) was used. Files containing the gene symbol for each protein along with the log transformation of the abundance ratio were evaluated for each treatment group. This software relates information on protein identification and abundance to biological networks using information from the literature. Results from these analyses indicate that several biological processes are involved in the response to LPS including Cu²⁺ homeostasis, apoptosis, endocytosis, and cell adhesion.

TABLE 22 below shows the biological processes identified by Metacore software. These processes were derived from our protein identifications and MetaCore's literature database which maps the biological network most likely associated with the identified proteins.

TABLE 22
Biological processes represented by Smoke regulated proteins.

| PROCESS | NO. | P-VALUE |
|---|---|---|
| copper ion homeostasis | 6 | 1.09E−09 |
| cell motility | 10 | 4.47E−07 |
| axonogenesis | 6 | 7.37E−06 |
| muscle contraction | 6 | 4.59E−04 |
| induction of apoptosis | 6 | 1.25E−03 |
| cell adhesion | 11 | 2.28E−03 |
| protein biosynthesis | 7 | 4.32E−03 |
| apoptosis | 8 | 1.04E−02 |
| transport | 16 | 2.14E−02 |

Proteins from smoke exposed animals were associated with copper ion homeostasis, cell motility and transport. Cell motility encompasses a class of proteins that promotes the inflammatory and immune response mediated by cytokines. These cytokines might be secreted by resident lung alveolar macrophages, lung epithelial and smooth muscle cells or by infiltrating leukocytes recruited from pulmonary circulation. The infiltrating leukocytes also express integrins, which facilitate their adherence to the intercellular adhesion molecules of the vascular endothelium before they migrate into the lung tissue space.

The transport process is represented by ATPases, which are involved in ATP degradation, not synthesis. This demonstrates that the cells are being deprived of ATP, potentially due to hypoxia, by the smoke exposure.

There is a decrease in transferrin levels in the Smoke group, which binds and transports iron and is synthesized by alveolar macrophages. A decrease in transferrin may cause an increase in “free” iron, which is the toxic form, and accumulation of iron in the lung has been demonstrated in smokers and patients with pulmonary diseases. Smoke exposure also causes a decrease in hemoglobin alpha and beta chains, which is responsible for blood oxygen transport.

The biological process identified for the Smoke+LPS group are similar to those seen for the microarray data of the combined treatment group (see TABLE 23). Again, cell motility emerges as the most significant process, most likely due to the more profound inflammatory infiltration noted by histopathology and by the cytology counts of macrophages and neutrophils. The SMOKE+LPS group had the highest immune cell counts of all three treatment groups, thus more significantly changed proteins associated with cell motility and recruitment. This synergistic damaging effect was also responsible for inducing the expression of a greater number of genes in the combined treatment group as opposed to the individual treatments, which is reflected in proteomics study by several proteins involved in the reorganization of the nucleosome assembly essential for active transcription.

TABLE 23
Biological processes represented by SMOKE + LPS group.

| PROCESS | NO. | P-VALUE |
|---|---|---|
| cell motility | 8 | 5.40E−05 |
| epidermis development | 5 | 6.39E−04 |
| protein biosynthesis | 8 | 1.50E−03 |
| protein modification | 5 | 9.79E−03 |
| protein folding | 5 | 1.68E−02 |
| metabolism | 6 | 2.26E−02 |
| negative regulation of cell proliferation | 5 | 3.78E−02 |
| cell proliferation | 7 | 4.28E−02 |

Systems Approach

Lung proteins that are associated with pathological responses in the lung following exposure to Smoke or Smoke+LPS were identified. It is now believed that the proteomics approach can differentiate potential differences among treatment groups, thereby increasing the confidence in handling these types of data for marker induction.

Using a global proteomics approach based on tandem- and FTICR mass spectrometry, we identified around 1200 proteins and demonstrated that around 300 of these have abundance changes attributable to one or more of the treatments. A number of proteins were up-regulated including DNA binding proteins, transcription factors, structural proteins, and proteins involved in host resistance. A notable example is pulmonary surfactant protein D which was up-regulated by a factor of 5 to 8 depending upon the treatment group.

Other proteins include cathepsin D (a protease) and cystatin B (a protease inhibitor) were up-regulated in the Smoke, and in the Smoke and LPS+Smoke treatment groups, respectively. These results indicate that an imbalance in protease activity can contribute to development of COPD, especially emphysema.

Additionally, approximately 25% of the proteins with altered abundance were down-regulated. Notable examples include catalase-1 and carbonyl reductase which are involved in detoxification of hydrogen peroxide and lipid peroxides, respectively. Hepatoma derived growth factor was the most dramatically altered protein in that it was down-regulated by more than 2 orders of magnitude. This effect was attributable to smoke exposure. This protein is nuclear with a DNA binding domain and is extracellular with growth factor activity.

The histopathological results indicated primarily neutrophil infiltrate in alveoli for LPS group, while the SMOKE and SMOKE+LPS groups had mixed inflammatory cell infiltrates consisting of neutrophils and macrophages. The BAL cytology showed that the LPS group had the highest neutrophil counts, whereas the SMOKE and SMOKE+LPS groups were dominated by macrophages (70:30).

Cytokine analysis showed that the LPS group displayed the greatest increases in BAL cytokines, while KC and TARC were highest in the SMOKE group. The SMOKE+LPS group had generally lower cytokine levels relative to LPS or SMOKE alone, indicating a potential immunosuppressive effect with combined exposure. TUNEL staining for apoptotic cells showed comparable cell counts in the LPS and SMOKE groups, with the highest counts in the SMOKE+LPS group.

The transcriptomic analysis demonstrated decreased heat shock proteins and increased immune and inflammatory response in all three groups; however, the SMOKE+LPS group had a reduced number of inflammation genes and networks than LPS alone.

There is shown by this data that there is a clear distinction between SMOKE- and LPS-induced inflammatory and immune response pathways from microarray data. There is shown by this data that the SMOKE+LPS animal model displays phenotypes and molecular signatures consistent with several proposed mechanisms of COPD: unchecked immune regulation of inflammatory response, calcium homeostasis imbalance, cell death versus proliferation imbalance affecting various cell types, protease activity, decreased macrophage function, and imbalance of oxidant to antioxidant potential.

Also, there are differential mechanisms of apoptosis suggested from microarray data, which are also shown by proteomic data findings. The data show that there are several peptide markers that are useful for exposure assessments. Also shown by this data are gene and protein markers which correlate with histopathological assessment of inflammatory effects.

### EXAMPLE IV

### Screening for Harm Reduction Products

The animal model as described herein is useful to test and compare products that normally have a harmful effect on a subject. In one example, the animal model is useful to test the reduction in harm caused by a tobacco product or other product that may, or is believed to, cause disease. In such example, different sets of the animal models are exposed to two or more products in order to compare which product has the least detrimental effect. The animal models are examined by at least screening for one or more markers as described herein to determine if any detrimental effects are being caused by the products. The different products are compared to determine which is a safer, or at least less harmful, product.

### EXAMPLE V

### Screening for Beneficial Products

The animal model as described herein is useful to test and compare products that normally have a beneficial effect on a subject. In one example, the animal model is useful to test whether a pharmaceutical, nutraceutical and/or homeopathic product has a beneficial effect, or prevents or minimized damage caused by harmful products that may, or are believed to, cause disease. In such example, different sets of the animal models are exposed to two or more products in order to compare which products have a beneficial effect. The animal models are examined by at least screening for one or more markers as described herein to determine if any beneficial effects are being caused by the products. The different products are compared to determine which is a safe and/or therapeutically effective product.

### EQUIVALENTS

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope, including the use of fragments or functional equivalents of the markers described herein.

Also, it is to be understood that all of the methods of the present invention may be performed entirely in vitro.

Throughout this specification, various aspects of this invention are presented in a range

Throughout this specification, various aspects of this invention are presented in a range format. It should be understood that the description in range format is merely for convenience and brevity and should not be construed as an inflexible limitation on the scope of the inventions. Accordingly, the description of a range should be considered to have specifically disclosed all the possible subranges as well as individual numerical values within that range. For example, description of a range such as from 1 to 5 should be considered to have specifically disclosed subranges (such as from 1 to 3, from to 4. etc), as well as individual numbers with that range (such as 1, 2, 3, 4, 5). This applies regardless of the breadth of the range. In addition, the fractional ranges are also included in the exemplified amounts that are described. In a non-limiting example, a range between 1-3 includes fractions such as 1.1, 1.2, 1.3, etc.

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

Sequence Information

The following TABLE 24 sequentially lists the SEQ ID. Nos. for the sequences described herein:

TABLE 24

| SEQ | | | | See | See | See |
|---|---|---|---|---|---|---|
| ID No. | ID_1 | ID_2 | Search_ID | Table | Table | FIGURE |
| 1 | Saa1 | | Saa1 | 14 | 15 | FIG. 7 |
| 2 | Mmp12 | | Mmp12 | 14 | | |
| 3 | AA467197 | | AA467197 | 14 | 15 | |
| 4 | Tnfrsf9 | | Tnfrsf9 | 14 | | |
| 5 | Hspa1a | | Hspa1a | 14 | 15 | |
| 6 | Marco | | Marco | 15 | | |
| 7 | Orm2 | | Orm2 | 15 | | |
| 8 | Ctsk | | Ctsk | 15 | | |
| 9 | Bub1b | | Bub1b | 24 | | |
| 10 | Cdc2a | | Cdc2a | 24 | | |
| 11 | Ctsb | | Ctsb | 24 | | |
| 12 | Tyrobp | | Tyrobp | 24 | | |
| 13 | BC024561 | | BC024561 | 24 | | |
| 14 | Top2a | | Top2a | 24 | | |
| 15 | Flt1 | | Flt1 | 24 | | |
| 16 | Ptgfr | | Ptgfr | 24 | | |
| 17 | Agtrl1 | | Agtrl1 | 24 | | |
| 18 | Sfn | | Sfn | 24 | | |
| 19 | Myh7 | | Myh7 | 24 | | |
| 20 | Plagl1 | | Plagl1 | 24 | | |
| 21 | Cacnb2 | | Cacnb2 | 24 | | |
| 22 | Soxl1 | | Soxl1 | 24 | | |
| 23 | Noxo1 | NM_027988 | NM_027988 | 14 | | |
| 24 | Saa3 | NM_011315 | NM_011315 | 14 | 15 | |
| 25 | Ly6i | NM_020498 | NM_020498 | 14 | 15 | |
| 26 | Vnn1 | NM_011704 | NM_011704 | 14 | | |
| 27 | Nrld1 | NM_145434 | NM_145434 | 14 | 15 | |
| 28 | Kif22 | NM_145588 | NM_145588 | 15 | | |
| 29 | Dtl | NM_029766 | NM_029766 | 15 | | |
| 30 | Melk | NM_010790 | NM_010790 | 15 | | |
| 31 | Orm1 | NM_008768 | NM_008768 | 15 | | |
| 32 | Kntc2 | NM_023294 | NM_023294 | 15 | | |
| 33 | Cd209b | NM_026972 | NM_026972 | 15 | | |
| 34 | Hspl10 | NM_013559 | NM_013559 | 15 | | |
| 35 | Lig1 | NM_010715 | NM_010715 | 16 | | |
| 36 | Cdca5 | NM_026410 | NM_026410 | 16 | | |
| 37 | Parvg | NM_022321 | NM_022321 | 16 | | |
| 38 | Ptgds2 | NM_019455 | NM_019455 | 16 | | |
| 39 | Asf1b | NM_024184 | NM_024184 | 16 | | |
| 40 | Mpeg1 | XM_924014 | XM_924014 | 16 | | |
| 41 | Kntc1 | NM_001042421 | NM_001042421 | 16 | | |
| 42 | Hist1h2ao | NM_178185 | NM_178185 | 16 | | |
| 43 | Mphosph1 | XM_973665 | XM_973665 | 16 | | |
| 44 | Adh6b | XM_975292 | XM_975292 | 16 | | |
| 45 | Pbk | NM_023209 | NM_023209 | 16 | | |
| 46 | Asahl | NM_025972 | NM_025972 | 16 | | |
| 47 | Pira2 | NM_011089 | NM_011089 | 16 | | |
| 48 | Cxcl1 | NM_008176 | NM_008176 | 14 | | |
| 49 | Slc26a4 | NM_011867 | NM_011867 | 14 | | |
| 50 | Has3 | NM_008217 | NM_008217 | 14 | | |
| 51 | Trem2 | NM_031254 | NM_031254 | 14 | 15 | |
| 52 | Ccl20 | NM_016960 | NM_016960 | 14 | | |
| 53 | Dlg7 | NM_144553 | NM_144553 | 16 | | |
| 54 | Gnmt | NM_010321 | NM_010321 | 16 | | |
| 55 | Hist2h3c2 | NM_054045 | NM_054045 | 16 | | |
| 56 | Atf5 | NM_030693 | NM_030693 | 16 | | |
| 57 | Kras | NM_021284 | NM_021284 | 16 | | |
| 58 | Hspa4 | NM_008300 | NM_008300 | 16 | | |
| 59 | Myst3 | XM_982598 | XM_982598 | 16 | | |
| 60 | Trim63 | NM_001039048 | NM_001039048 | 16 | | |
| 61 | Cpn1 | NM_030703 | NM_030703 | 16 | | |
| 62 | 1459552_at | AK013071 | AK013071 | 16 | | |
| 63 | 1439284_at | AW909503 | AW909503 | 16 | | |
| 64 | 1438704_at | BG817292 | BG817292 | 16 | | |
| 65 | 1429510_at | AW537770 | AW537770 | 16 | | |
| 66 | 1448612_at | NM_018754 | NM_018754 | 16 | | |
| 67 | 1435194_at | BB404047 | BB404047 | 16 | | |
| 68 | NCL | NP_035010 | NP_035010 | 21 | | |
| 69 | COL5A1 | NP_056549 | NP_056549 | 21 | | |
| 70 | CFL1 | NP_031713 | NP_031713 | 21 | | |
| 71 | RPS28 | AAC97967 | AAC97967 | 21 | | |
| 72 | PSME1 | BAB47403 | BAB47403 | 21 | | |
| 73 | RPLP2 | NP_080296 | NP_080296 | 21 | | |
| 74 | DPYSL2 | NP_034085 | NP_034085 | 21 | | |
| 75 | ALDH2 | AAH05476 | AAH05476 | 21 | | |
| 76 | SPNB2 | AAH92544 | AAH92544 | 21 | | |
| 77 | VAPA | NP_038961 | NP_038961 | 21 | | |
| 78 | RAC1 | AAH51053 | AAH51053 | 21 | | |
| 79 | ANXA2 | AAH04659 | AAH04659 | 21 | | |
| 80 | XP_915382 | | XP_915382 | 19 | | |
| 81 | XP_925763 | | XP_925763 | 19 | | |
| 82 | NP_542126 | | NP_542126 | 19 | 20 | |
| 83 | XP_485004 | | XP_485004 | 19 | 20 | |
| 84 | NP_032016 | | NP_032016 | 19 | 20 | |
| 85 | XP_926145 | | XP_926145 | 19 | | |
| 86 | NP_031457 | | NP_031457 | 19 | | |
| 87 | NP_031789 | | NP_031789 | 19 | 20 | |
| 88 | NP_059066 | | NP_059066 | 19 | 20 | |
| 89 | NP_033186 | | NP_033186 | 19 | 20 | |
| 90 | NP_058089 | | NP_058089 | 20 | | |
| 91 | NP_062264 | | NP_062264 | 20 | | |
| 92 | NP_032251 | | NP_032251 | 20 | | |
| 93 | XP_920646 | | XP_920646 | 20 | | |
| 94 | NP_034578 | | NP_034578 | 20 | | |
| 95 | NP_058020 | | NP_058020 | 20 | | |
| 96 | CALR | CAG33351 | CAG33351 | 21 | | |
| 97 | Chmp4b | AAH59279 | AAH59279 | 21 | | |
| 98 | Ubap2l | AAH50910 | AAH50910 | 21 | | |
| 99 | Ppia | CAG32988 | CAG32988 | 21 | | |
| 100 | WBSCR1 | AAF75557 | AAF75557 | 21 | | |
| 101 | GSTM1 | CAG46666 | CAG46666 | 21 | | |
| 102 | Fusip1 | AAH83082 | AAH83082 | 21 | | |
| 103 | ALDH1A1 | AAH44729 | AAH44729 | 21 | | |
| 104 | PRDX6 | NP_031479 | NP_031479 | 21 | | |
| 105 | Vimentin | CAA39807 | CAA39807 | | | FIG. 15 |
| 106 | AHNAK | AAQ97238 | AAQ97238 | | | FIG. 15 |
| 107 | periaxin isoform L | NP_932165 | NP_932165 | | | FIG. 15 |
| 108 | 1700022C02Rik | NM_025495 | NM_025495 | 15 | 16 | |
| 109 | A330102K23Rik | NM_153409 | NM_153409 | 16 | | |
| 110 | E230015L20Rik | NM_177111 | NM_177111 | 16 | | |
| 111 | E230025E14Rik | AK054177 | AK054177 | 16 | | |
| 112 | 2610318C08Rik | NM_181589 | NM_181589 | 16 | | |
| 113 | C330027C09Rik | NM_172616 | NM_172616 | 16 | | |
| 114 | 5730507H05Rik | XM_975418 | XM_975418 | 16 | | |
| 115 | 2810417H13Rik | NM_026515 | NM_026515 | 16 | | |
| 116 | Apolipoprotein A-1 | AAD34604 | AAD34604 | | | FIG. 15 |
| 117 | Annexin A1 | AAH35993 | AAH35993 | | | FIG. 15 |
| 118 | Gbeta3 | NP_067642 | NP_067642 | | | FIG. 15 |
| 119 | Serpin | AAB35530 | AAB35530 | | | FIG. 15 |
| 120 | Fibrillar collagen | AAN41263 | AAN41263 | | | FIG. 15 |
| 121 | Gxcl5 | NM_002994 | NM_002994 | 14 | | FIG. 7 |
| 122 | Zranb3 | NM_032143 | NM_032143 | 14 | | FIG. 7 |
| 123 | Eraf | NM_016633 | NM_016633 | | | FIG. 7 |
| 124 | Fshprh1 | NM_006733 | NM_006733 | 15 | | FIG. 7 |
| 125 | Ccnb-rs1 | NM_031966 | NM_031966 | | | FIG. 7 |
| 126 | Tnfrsf10b | EF064712 | EF064712 | | | FIG. 7 |
| 127 | Cxcl9 | NM_002416 | NM_002416 | 15 | | FIG. 7 |
| 128 | Cxcl11 | NM_005409 | NM_005409 | | | FIG. 7 |
| 129 | LOC639389 | XP_920894 | XP_920894 | 21 | | |
| 130 | CyclinN | AAH26187 | AAH26187 | | | FIG. 15 |

### REFERENCES

The references discussed above and the following references, to the extent that they provide exemplary procedural or other details supplementary to those set forth herein, are specifically incorporated herein by reference.
Abram, M., E. Strittmatter, M. Monroe, J. N. Adkins, J. Hunter, J. H. Miller, D. L. Springer. 2005. Proteomics, 5(7) 1815-1826.
Adkins, J. N., Monroe, M., Auberry, K. J., Shen, Y., Jacobs, J. M., Camp, D. G. III, Vitzthum F., Rodland K. D., Zangar, R. C., Smith R. D., Pounds, J G. 2005. Proteomics 5, 3454-3466.
Allan, S M., Tyrrell P J, Rothwell N J 2005. “Interleukin-1 and neuronal injury” Nature Reviews Immunology 5 (2005) 629-40.
Banfi, B., Clark, R. A., Steger, K., and Krause, K. H. 2003. Two novel proteins activate superoxide generation by the NADPH oxidase NOX1. J. Biol. Chem. 278:3510-3.
Barreda D R, Hanington P C, Belosevic M. Regulation of myeloid development and function by colony stimulating factors. Developmental and Comparative Immunology 28 (2004) 509-55.
Belperio J A, Dy M, Murray L, Burdick M D, Xue Y Y, Strieter R M, Keane M P. The Role of the Th2 CC Chemokine Ligand CCL17 in Pulmonary Fibrosis. J. Immunol. 173 (2004) 4692-469.
Cavagna G., Foa, V. and Vigliani, E. C. 1969. Effects in man and rabbits of inhalation of cotton dust or extracts and purified endotoxins. Br. J. Ind. Med. 26(4):314-21.
Cawston, T., Carrere, S., Catterall, J., Duggleby, R., Elliott, S., Shingleton, B., and Rowan, A. 2001. Matrix metalloproteinases and TIMPs: properties and implications for the treatment of chronic obstructive pulmonary disease. Novartis Found Symp. 234:205-18. vivo contractile properties of the vastus lateralis muscle in males with COPD. Eur. Respir. J. 21:273-8.
Doniger, S. W., Salomonis, N., Dahlquist, K. D., Vranizan, K., Lawlor, S. C., and Conklin, B. R. 2003. MAPPFinder: using Gene Ontology and GenMAPP to create a global gene-expression profile from microarray data. Genome Biol. 4:R7.
Fostera P S, Martinez-Moczygemba M, Huston D P, Corry D B. Interleukins-4, -5, and -13: emerging therapeutic targets in allergic disease. Pharmacology & Therapeutics 94 (2002), 253-264.
Granucci, F., Petralia, F., Urbano, M., Citterio, S., Di Tota, F., Santambrogio, L., and Ricciardi-Castagnoli, P. 2003. The scavenger receptor MARCO mediates cytoskeleton rearrangements in dendritic cells and microglia. Blood 102:2940-7.
Greenberger, M. J., Strieter, R. M., Kunkel, S. L., Danforth, J. M., Laichalk, L. L., McGillicuddy, D. C., and Standiford, T. J. 1996. Neutralization of macrophage inflammatory protein-2 attenuates neutrophil recruitment and bacterial clearance in murine Klebsiella pneumonia. J. Infect. Dis. 173:159-65.
Gupta, S., and Knowlton, A. A. 2005. HSP60, Bax, apoptosis and the heart. J. CellMol. Med. 9:51-8.
Hasday, J. D., Bascom, R., Costa, J. J., Fitzgerald, T., and Dubin, W. 1999. Bacterial endotoxin is an active component of cigarette smoke. Chest. 115:829-35.
Hautamaki, R. D., Kobayashi, D. K., Senior, R. M., and Shapiro, S. D. 1997. Requirement for macrophage elastase for cigarette smoke-induced emphysema in mice. Science 277:2002-4.
Hill, A. T., Campbell, E. J., Hill, S. L., Bayley, D. L., and Stockley, R. A. 2000. Association between airway bacterial load and markers of airway inflammation in patients with stable chronic bronchitis. Am. J. Med. 109:288-95.
Jacobelli M, Rohwer F, Shanahan P, Quiroz J A, McGuire K L. IL-2-Mediated Cell Cycle Progression and Inhibition of Apoptosis Does Not Require NF-kB or Activating Protein-1 Activation in Primary Human T Cells. The Journal of Immunology, 162 (1999) 3308-3315.
Joos, L., He, J. Q., Shepherdson, M. B., Connett, J. E., Anthonisen, N. R., Pare, P. D., and Sandford, A. J. 2003. The role of matrix metalloproteinase polymorphisms in the rate of decline in lung function. Hum. Mol. Genet. 11:569-76.
Kopydlowski K M, Salkowski C A, Cody M J, van Rooijen N, Major J, Hamilton T A, Vogel S N. Regulation of Macrophage Chemokine Expression by Lipopolysaccharide In Vitro and In Vivo. J. Immunol, 163 (1999) 1537-1544.
Laan, M., Bozinovski, S., and Anderson, G. P. 2004. Cigarette smoke inhibits lipopolysaccharide-induced production of inflammatory cytokines by suppressing the activation of activator protein-1 in bronchial epithelial cells. J. Immunol. 176:4164-70.
Larson, M. A., Weber, A., Weber, A. T., McDonald, T. L. 2005. Differential expression and secretion of bovine serum amyloid A3 (SAA3) by mammary epithelial cells stimulated with prolactin or lipopolysaccharide. Vet. Immunol. Immunopathol. 107:255-64.
MacNee, W. 2000. Oxidants/Antioxidants and COPD. Chest 117(5 Suppl 1): 303S-17S.
MacNee, W. 2005. Pulmonary and systemic oxidant/antioxidant imbalance in chronic obstructive pulmonary disease. Proc. Am. Thorac. Soc. 2:50-60.
Martin, T. R. 2000. Recognition of bacterial endotoxin in the lungs. Am. J. Respir. Cell Mol. Biol. 23:128-132.
Mattson, J. P., and Martin, J. C. 2005. Emphysema-induced reductions in locomotory skeletal muscle contractile function. Exp. Physiol. 90:519-25.
Michel, O., Duchateau, J., and Sergysels, R. 1989. Effect of inhaled endotoxin on bronchial reactivity in asthmatic and normal subjects. J. Appl. Physiol. 66:1059-64.
Michel, O., Duchateau, J., Plat, G., Cantinieaux, B., Hotimsky A., Gerain, J., and Sergysels, R. 1995. Blood inflammatory response to inhaled endotoxin in normal subjects. Clin. Exp. Allergy. 25:73-9.
Nevalainen, M., Raulo, S. M., Brazil, T. J., Pine R. S., Sorsa, T., McGorium, B. C., and Maisi, P. 2002. Inhalation of organic dusts and lipopolysaccharide increases gelatinolytic matrix metalloproteinases (MMPs) in the lungs of heaves horses. Equine. Vet. J. 34:150-5.
Nesvizhskii Al, Keller A, Kolker E, Aebersold R. 2003. A statistical model for identifying proteins by tandem mass spectrometry. Anal Chem 75: 4646-4658.
Nokolsky, Y., Ekins, S., Nikolskaya, T., and Burgrim, A. 2005. A novel method for generation of signature networks as markers from complex high throughput data. Toxicol. Lett. 158:20-9.
Ottenheijm, C. A., Heunks, L. M., Sieck, G. C., Zhan, W. Z., Jansen, S. M., Degens. H., de Boo, T., and Dekhuijzen, P. N. 2005. Diaphragm dysfunction in chronic obstructive pulmonary disease. Am. J. Respir Crit. Care Med. 172:200-5.
Petro T M. Modulation of IL-12 p35 and p40 promoter activity by smokeless tobacco extract is associated with an effect upon activation of NF-nB but not IRF transcription factors. International Immunopharmacology 3 (2003) 735-745.
Psarras, S., Caramori, G., Controli, M., Papadopoulos, N., and Papi, A. 2005. Oxidants in asthma and in chronic obstructive pulmonary disease (COPD). Curr. Pharm. Des. 11:2053-62.
Qvarfordt, I., Riise, G. C., Andersson, B. A., and Larsson, S. 2000. Lower airway bacterial colonization in asymptomatic smokers and smokers with chronic bronchitis and recurrent exacerbations. Respir. Med. 94:881-7.
Rahman, I. 2005. The role of oxidative stress in the pathogenesis of COPD: implications for therapy. Treat. Respir. Med. 4:175-200.
Sethi, S., Muscarella, K., Evans, N., Klingman, K. L., Grant, B. J., and Murphy, T. F. 2000. Airway inflammation and etiology of acute exacerbations of chronic bronchitis. Chest 118:1557-65.
Shibata, Y., Berclaz, P. Y., Chroneos, Z. C., Yoshida, M., Whitsett, J. A., and Trapnell, B. C. 2001. GM-CSF regulates alveolar macrophage differentiation and innate immunity in the lung through PU.1. Immunity 15:557-67.
Smid, T., Heederik, D, Houba, R. and Quanjer P. H. 1994. Dust- and endotoxin-related acute lung function changes and work-related symptoms in workers in the animal feed industry. Am. J. Ind. Med. 25:877-88.
Snijders A, Hilkens C M, van der Pouw Kraan T C, Engel M, Aarden L A, Kapsenberg M L. Regulation of bioactive IL-12 production in lipopolysaccharide-stimulated human monocytes is determined by the expression of the p35 subunit. J. Immunol. 156 (3) 1996 1207-12.
Spond, J., Billah M. M., Chapman, R. W., Egan, R. W., Hey, J. A., House, A., Kreutner, W., and Minnicozzi, M. 2004. The role of neutrophils in LPS-induced changes in pulmonary function in conscious rats. Pulm. Pharmacol. Ther. 17:133-40.
Strittmatter E F K L, Petritis K, Mottaz H M, Anderson G A, Shen Y, Jacobs J M, Camp D G, Smith R D (2004) Application of Peptide Retention Time Information in a Discriminant Function for Peptide Identification by Tandem Mass Spectrometry. Journal of Proteome Research 3: 760-769.
Toward, T. J., and Broadley, K. J. 2002. Goblet cell hyperplasia, airway function, and leukocyte infiltration after chronic lipopolysaccharide exposure in conscious Guinea pigs: effects of rolipram and dexamethasone. J. Pharmacol. Exp. Ther. 302:814-21.
van der Vaart, H., Postma, D. S., Timens, W., Hylkema, M. N., Willeinse, B. W. M., Boezen, H. M., Vonk, J. M., de Reus, D. M., Kauffinan, H. F., and ten Hacken, N. H. T. 2005a. Acute effects of cigarette smoking on inflammation in healthy intermittent smokers. Respir. Res. 6:22.
van der Vaart, H., Postma, D. S., Timens, W., and ten Hacken, N. H. T. 2005b. Acute effects of cigarette smoke on inflammation and oxidative stress: a review. Thorax 59:713-21.
Vernooy, J. H., Denterner, M. A., van Suylen, R. J., Buurman, W. A., and Wouters, E. F. 2002. Intratracheal instillation of lipopolysaccharide in mice induces apoptosis in bronchial epithelial cells: no role for tumor necrosis factor-alpha and infiltrating neutrophils. Am. J. Respir. Cell Mol. Biol. 24:569-76.
Vernooy, J. H., Denterner, M. A., van suylen, R. J., Buurman, W. A., and Wouters, E. F. 2002. Long-term intratracheal lipopolysaccharide exposure in mice results in chronic lung inflammation and persistent pathology. Am. J. Respir. Cell Mol. Biol. 26:152-9.
Vogelzang, P. F., Van Der Gulden, J. W., Folgering, H., Kolk, J. J., Heederik, D., Preller, L., Tielen, M. J., and Van Schayck, C. P. 1998. Am. J. Respir. Crit. Care Med. 157(1):15-8.
Washburn, M. P., Ulaszek, R., Deciu, C., Schieltz, D. M., et al., 2002. Anal Chem, 74: 1650-1657.
Wong M M, Fish E N. Chemokines: attractive mediators of the immune response. Seminars in Immunology 15 (2003) 5-14.
Young, H. D. 1962. Statistical Treatment of Experimental Data, (McGraw-Hill, New York, p 100).

1. A method for producing a non-human animal model for assessment of at least one COPD-related disease, the method comprising:
i) exposing the animal to repeated doses of at least one chemical found in smoke and at least one toxin including LPS.

2. A method of claim 1, further comprising
ii) collecting one or more selected samples from the animal; and
iii) comparing the collected sample to one or more indicia of potential COPD initiation or development.

3. The method of claim 2, wherein the step iii) includes:
a) comparing pathology changes in the animal, and
b) identifying a level of infiltration of the chemical and toxin in respiratory system tissue of the animal with one or more of increased inflammatory response, increased macrophage activity, and altered level of neutrophil infiltration.

4. The method of claim 1, wherein the step i) includes alternating exposures of the chemical and the toxin in a manner sufficient to initiate a COPD-related disease response in the animal model.

5. The method of claim 1, wherein the step i) includes:
a) repeating doses of the smoke chemical for at least one period during a number of successive days, followed by repeating doses of the LPS toxin for at least one period during successive days; and, optionally,
b) repeating the previous step at least two additional times.

6. The method of claim 1, wherein the step a) includes controlling a delivered amount of the LPS toxin by producing inhalable quantities of the LPS toxin and, optionally, combining the inhalable LPS toxin with heated diluted air.

7. The method of claim 2, wherein in response to data generated by the step iii), the method further includes at least a further step:
iv) adjusting exposure of the animal to at least one of the smoke chemical and, optionally, LPS.

8. The method of claim 2, wherein the step iii) is performed simultaneously with step i), and step iii).

9. The method of claim 2, wherein the steps ii) and iii) include one or more of:
a) blood as the sample analyzed for one or more of carboxyhemoglobin (COHb), nicotine and cotinine;
b) lung tissue as the sample analyzed for at least one of bronchoalveolar lavage (BAL), histopathology or immunohistology; and
c) BAL fluid (BALF) as the sample analyzed for at least one of enzymes, total protein, cytology and cytokines.

10. The method of claim 2, wherein the step iii) includes analysis by one or more of:
hierarchical clustering, signature network construction, mass spectroscopy proteomic analysis, surface plasmon resonance, linear statistical modeling, partial least squares discriminant analysis, and multiple linear regression analysis.

11. The method of claim 1, wherein the chemical comprises one or more of:
tobacco, a non-tobacco smoking product, an actively oxidizing material producing an inhalable particle, an inhalable chemical, a vaporized material, a droplet of material, and an inhalable particle.

12. The method of claim 1, wherein the chemical comprises one or more chemicals found in cigarette tobacco smoke.

13. A non-human animal model obtainable by a method of claim 1, wherein the model is for assessing one or more metabolic pathways that contribute to at least one of initiation, progression, severity, pathology, aggressiveness, grade, activity, disability, mortality, morbidity, disease sub-classification or other underlying pathogenic or pathological feature of at least one COPD-related disease,
wherein at least one of the following biological or chemical processes occurs in the animal model after exposure of the animal to the at least one chemical found in smoke and the at least one toxin including LPS:
i) decreased heat shock response and/or chaperone activity,
ii) altered immune and inflammatory response,
iii) increased cell proliferation,
iv) unchecked immune regulation of inflammatory response,
v) calcium homeostasis imbalance,
vi) cell death versus proliferation imbalance affecting several cell types,
vii) protease activity,
viii) decreased macrophage function, and
ix) imbalance of oxidant to antioxidant potential.

14. The animal model of claim 13, wherein the animal's lungs are infiltrated with at least one of the LPS toxin and at least one of the smoke chemicals, whereby an inflammatory response occurs.

15. A non-human animal model obtainable by a method of claim 1, wherein the model is for assessing at least one COPD-related disease, and wherein an expression level of one or more markers, or a functional equivalent thereto, has been increased or decreased in the animal model after exposure of the animal to the at least one chemical found in smoke and the at least one toxin including LPS, wherein the marker comprises one or more of:
i) one or more of the BAL cytokines, or fragments thereof, as shown in TABLE 8, or fragments or functional equivalents thereof;
ii) one or more genes, or fragments thereof, as shown in at least one of TABLES 14, 15 and 16, or fragments or functional equivalents thereof;
iii) one or more genes, or fragments or functional equivalents thereof, encoding for a protein, or fragment or fragments or functional equivalents thereof, as shown in at least one of TABLES 19, 20 and 21; and,
iv) one or more genes, or fragments or functional equivalents thereof, encoding for a protein, or fragment or functional equivalents thereof, that produces one or more of the biological or chemical processes as shown in at least one of TABLES 17, 18, 22 and 23.

16. The animal model of claim 15, wherein the marker comprises one or more of the cytokines, or fragments or functional equivalents thereof, as shown in TABLE 8.

17. The animal model of claim 15, wherein the marker comprises one or more of markers IL-12p40, G-CSF, RANTES, IL-2, IL-5 and IL12p70, or fragments or functional equivalents thereof.

18. The animal model of claim 15, wherein one or more of markers IL-12p40, G-CSF and RANTES, or fragments or functional equivalents thereof, are up-regulated, and one or more of markers IL-2, IL-5 and IL12p70, or fragments or functional equivalents thereof, are down-regulated.

19. The animal model of claim 15, wherein the marker comprises one or more of the genes, or fragments or functional equivalents thereof, as shown in TABLE 14.

20. The animal model of claim 15, wherein the marker comprises one or more of: Saa1 [SEQ ID No. 1], Mmp12 [SEQ ID No. 2], heat shock protein [SEQ ID No. 5], or fragments or functional equivalents thereof.

21. The animal model of claim 15, wherein the marker comprises one or more of the genes, or fragments or functional equivalents thereof, as shown in TABLE 15.

22. The animal model of claim 15, wherein the marker comprises one or more of Saa1 [SEQ ID No. 1], heat shock protein [SEQ ID No. 5], or fragments or functional equivalents thereof.

23. The animal model of claim 15, wherein the marker comprises one or more of the genes, or fragments or functional equivalents thereof, as shown in TABLE 16.

24. The animal model of claim 15, wherein the marker comprises one or more of the proteins, or fragments or functional equivalents thereof, genes, or fragments or functional equivalents thereof, encoding for the proteins that produce one or more of the biological or chemical processes, as shown in TABLE 17.

25. The animal model of claim 15, wherein the marker comprises one or more of the genes, or fragments or functional equivalents thereof, related to an immune response or inflammatory response.

26. The animal model of claim 15, wherein the marker comprises one or more of the proteins, or fragments or functional equivalents thereof, genes, or fragments or functional equivalents thereof, encoding for the proteins that produce one or more of the biological or chemical processes, as shown in TABLE 18.

27. The animal model of claim 15 s, wherein the marker comprises one or more of the proteins, or fragments or functional equivalents thereof, genes, or fragments or functional equivalents thereof, encoding for the proteins that produce one or more of the following biological or chemical processes:
mitosis; cell division and cell cycle control.

28. The animal model of claim 15, wherein the marker comprises one or more of the proteins, or fragments or functional equivalents thereof, genes, or fragments or functional equivalents thereof, encoding for the proteins that produce one or more of the following biological or chemical processes:
muscle development; muscle contraction; calcium ion homeostasis and lipid metabolism.

29. The animal model of claim 15, wherein the marker comprises one or more of the proteins, or fragments or functional equivalents thereof, as shown in TABLE 19.

30. The animal model claim 15, wherein the marker comprises one or more of the proteins, or fragments or functional equivalents thereof, as shown in TABLE 20.

31. The animal model of claim 15, wherein the marker comprises one or more of surfactant protein D [SEQ ID No. 89], haptoglobin [SEQ ID No. 88], or fragments or functional equivalents thereof.

32. The animal model of claim 15, wherein the marker comprises one or more of the proteins, or fragments or functional equivalents thereof, encoding for the proteins as shown in TABLE 21.

33. The animal model of claim 15, wherein the marker comprises one or more of: nucleolin (NCL) [SEQ ID No. 68], calreticulin (CALR) [SEQ ID No. 96], procollagen, type V, alpha 1 (Col5A1) [SEQ ID No. 69], or fragments or functional equivalents thereof.

34. The animal model of claim 15, wherein the marker comprises one or more of the proteins, or fragments or functional equivalents thereof, that produces one or more of the biological or chemical processes as shown in TABLE 22.

35. The animal model of claim 15, wherein the biological or chemical processes encoded by the marker comprises one or more of:
copper ion homeostasis, cell motility and transport.

36. The animal model of claim 15, wherein the marker comprises one or more of the proteins, or fragments or functional equivalents thereof, produces one or more of the biological or chemical processes as shown in TABLE 23.

37. The animal model of claim 15, wherein the biological or chemical processes encoded by the marker comprises one or more of:
cell motility and reorganization of the nucleosome assembly.

38. The animal model of claim 15, wherein the marker comprises one or more of the up-regulated genes or down-regulated genes, or fragments or functional equivalents thereof, as shown in FIG. 7.

39. The animal model of claim 15, wherein the marker comprises one or more of:
follicle stimulating primary response gene 1 (1fshprh1) [SEQ ID No. 124], cyclin b1 related sequence (ccnb1-rs1) [SEQ ID No. 125], tumor necrosis family receptor superfamily member 10b (tnfrsf10b) [SEQ ID No. 126], or fragments or functional equivalents thereof.

40. The animal model of claim 15, wherein the marker comprises one or more of:
chemokine (C-X-C motif) ligand 5 (Cxc15) [SEQ ID No. 121], zinc finger RAN-binding domain containing 3 (Zranb3) [SEQ ID No. 122], erythroid associated factor (Eraf) [SEQ ID No. 123], or fragments or functional equivalents thereof.

41. The animal model of claim 15, wherein the marker comprises one or more of:
Cxcl9 [SEQ ID No. 127], Saa1 [SEQ ID No. 1], Cxcl11 [SEQ ID No. 128], or fragments or functional equivalents thereof.

42. The animal model of claim 15, wherein the marker comprises one or more of the up-regulated proteins or down-regulated proteins, or fragments or functional equivalents thereof, as shown in FIG. 15.

43. The animal model of claim 15, wherein the marker comprises one or more of:
Vimentin [SEQ ID No. 105], AHNAK nucleoprotein isoforms 1 [SEQ ID No. 106], periaxon isoform L [SEQ ID No. 107], or fragments or functional equivalents thereof.

44. The animal model of claim 15, wherein the marker comprises one or more of:
serine or cysteine peptidase inhibitor, Glade A member 1D (Serpin) [SEQ ID No. 119], cyclin fold protein 1 (CyclinN) [SEQ ID No. 130], procollagen type I, alpha 1 (fibrillar collagen), [SEQ ID No. 120], or fragments or functional equivalents thereof.

45. The animal model of claim 15, wherein the marker comprises one or more of:
Apoplipoprotein A-1 [SEQ ID No. 116], Annexin A-1 [SEQ ID No. 117], and Gbeta3 [SEQ ID No. 118], or fragments or functional equivalents thereof.

46. The animal model of claim 15 wherein the marker comprises one or more of the up-regulated genes or down-regulated genes, or fragments or functional equivalents thereof, as shown in FIG. 12.

47. The animal model of claim 15, wherein the marker comprises one or more of p22-phox, p47-phox and gp91-phox, or fragments or functional equivalents thereof.

48. The animal model of claim 15, wherein the marker comprises one or more of the up-regulated genes or down-regulated genes, or fragments or functional equivalents thereof, as shown in FIG. 13.

49. The animal model of claim 15, wherein the marker comprises one or more of cardiac troponin I (TNNI3), cardiac troponin T (TNNT2) and Actin, or fragments or functional equivalents thereof.

50. The animal model of claim 15, wherein the marker comprises one or more of the genes, or fragments or functional equivalents thereof, related to muscle development or muscle contraction.

51. The animal model of claim 15, wherein the marker comprises one or more of Mmp12 [SEQ ID No. 2], NOXO1 [SEQ ID No. 22], the serum amyloid group including Saa1 [SEQ ID No. 1], Saa2 and Saa3 [SEQ ID No. 24], MARCO [SEQ ID No. 6], or fragments or functional equivalents thereof.

52. The animal model of claim 15, wherein the marker comprises one or more of up-regulated markers including pulmonary surfactant protein D, cathepsin D, cystatin B; one or more of down-regulated markers including catalase-1, carbonyl reductase, hepatoma derived growth factor, or fragments or functional equivalents thereof.

53. The animal model of claim 15, wherein the marker comprises one or more of the genes, or fragments or functional equivalents thereof, as shown in TABLE 24.

54. A method of assessing the potential of at least one chemical found in smoke for an ability to initiate a COPD-related disease response in an animal model of claim 15, the method providing:
1) measuring one or more of up- or down-regulated markers (or fragments thereof) after exposure of the animal to one or more of: i) at least one smoke chemical, and ii) lipopolysaccharide (LPS) in amounts sufficient to initiate a COPD-related disease response in the animal;
2) determining whether at least one of the up- or down-regulated markers has the ability to initiate a COPD-related disease response.

55. The method of claim 54, wherein the chemical comprises one or more found in:
tobacco, a non-tobacco smoking product, an actively oxidizing material producing an inhalable particle, an inhalable chemical, a vaporized material, a droplet of material, and an inhalable particle.

56. The method of claim 55, wherein the chemical comprises one or more chemicals found in cigarette tobacco smoke.

57. A composition that induces a COPD-related disease response in an animal model of claim 15, wherein the composition includes, as an active ingredient:
i) one or more of the BAL cytokines, or fragments or functional equivalents thereof, as shown in TABLE 8;
ii) one or more genes, or fragments or functional equivalents thereof, as shown in at least one of TABLES 14, 15 and 16;
iii) one or more genes, or fragments or functional equivalents thereof, encoding for a protein, or fragments or functional equivalents thereof as shown in at least one of TABLES 19, 20 and 21; and,
iv) one or more genes, or fragments thereof, encoding for a protein, or fragments or functional equivalents thereof, that produces one or more of the biological or chemical processes as shown in at least one of TABLES 17, 18, 22 and 23.

58. A method of screening for a therapeutic agent for treatment of at least one COPD-related disease comprising the steps of:
1) assaying for an expression level of a marker in a sample obtained from the animal model of claim 15, wherein the marker comprises one or more of:
i) one or more of the BAL cytokines, or fragments or functional equivalents thereof, as shown in TABLE 8;
ii) one or more genes, or fragments or functional equivalents thereof, as shown in at least one of TABLES 14, 15 and 16;
iii) one or more genes, or fragments thereof, encoding for a protein, or fragments or functional equivalents thereof, as shown in at least one of TABLES 19, 20 and 21; and,
iv) one or more genes, or fragments or functional equivalents thereof, encoding for a protein, or fragments or functional equivalents thereof, that produces one or more of the biological or chemical processes as shown in at least one of TABLES 17, 18, 22 and 23; and
2) comparing the expression levels assayed to that in a control with which the therapeutic agent has not been contacted.

59. The method of claim 58, wherein the sample comprises one or more of blood, plasma, serum, urine, saliva, bronchoalveolar lavage (BAL), exhaled breath, or exhaled breath condensate.

60. The method of claim 58 wherein the sample comprises lung tissue or other tissue of the respiratory system.

61. The method of claim 58, wherein the assaying step (1) includes:
a) determining an initial level of one or more markers in a first sample from the animal,
b) determining a subsequent level of the one or more markers in a second sample from the animal after administration of the candidate therapeutic agent; and
c) determining whether the subsequent level of the one or more markers in the sample is higher or lower than the initial level of the marker in the first sample.

62. The method of claim 61, wherein the second sample is obtained at least about six hours after administering the candidate therapeutic agent.

63. The method of claim 61, wherein the second sample is obtained no more than about six months after administering the candidate therapeutic agent.

64. The method of claim 61 wherein the candidate therapeutic agent comprises one or more of: pharmaceutical compositions, nutraceutical compositions, and homeopathic compositions.

65. A method of assessing the effectiveness of a therapy to prevent, diagnose and/or treat at least one COPD-related disease comprising:
1) subjecting the animal model of claim 15 to a regimen whose effectiveness is being assessed, and
2) determining the level of effectiveness of the treatment being tested in treating or preventing the COPD-related disease.

66. The method of claim 65, wherein the therapy being assessed is for use in a human subject.

67. The method according to claim 65, wherein the method is neither a method for the treatment of the human or animal body by surgery or therapy nor a diagnostic method practiced on the human or animal body.

68. A method of providing a non-human animal model for at least one COPD-related disease complication, the method comprising exposing to a non-human animal at least one chemical found in smoke and lipopolysaccharide (LPS) in an amount sufficient to induce the at least one complication in the animal.

69. The method of claim 68, wherein the complication manifests in the animal at least about a month earlier than that in an available animal model not exposed to the at least one chemical and LPS.

70. The method of claim 68, wherein the COPD-related disease complication manifests in the animal at least about 3 weeks after exposure.

71. The method of claim 68, wherein the animal model develops the at least one COPD-related disease complication in the absence of severe acute morbidity.

72. A method of screening for a therapeutic agent useful for treating or preventing a COPD-related disease complication, comprising:
1) providing, by the method of claim 68, a test animal and a substantially identical control animal;
2) maintaining the test animal and the control animal under conditions appropriate for development of at least one COPD-related disease complication in the control animal;
3) assessing the at least one COPD-related disease complication in the test animal and the control animal; and,
4) comparing the severity and/or onset of the COPD-related disease complication in the test animal with that of the control animal,
wherein reduced severity and/or delay in the onset of the COPD-related disease complication in the test animal indicates that the candidate agent is the therapeutic agent useful for treating or preventing the COPD-related disease complication.

73. The method of claim 68, wherein the method is neither a method for the treatment of the human or animal body by surgery or therapy nor a diagnostic method practiced on the human or animal body.

74. An animal model that has been sensitized with at least one chemical found in smoke and at least one toxin including LPS over a period of about 3 weeks, wherein the animal exhibits at least one symptom of a COPD-related disease in a shorter period of time than a conventional animal.

75. The animal model of claim 74, wherein the animal develops COPD-disease related symptoms within at least about 3 weeks.

76. The animal model of claim 75, wherein the animal develops COPD-disease related symptoms sufficient for evaluating the effectiveness of a therapeutic agent within at least about 3 weeks.

77. The animal model of claim 76, wherein the at least one symptom includes exhibiting at least one or more of at least one of the following biological or chemical processes:
i) decreased heat shock response and/or chaperone activity,
ii) altered immune and inflammatory response,
iii) increased cell proliferation,
iv) unchecked immune regulation of inflammatory response,
v) calcium homeostasis imbalance,
vi) cell death versus proliferation imbalance affecting several cell types,
vii) protease activity,
viii) decreased macrophage function, and
ix) imbalance of oxidant to antioxidant potential.

78. The animal model of claim 77, wherein the animal's lungs are infiltrated with at least one of the LPS toxin and at least one of the smoke chemicals, whereby an inflammatory response occurs.

79. The animal model of claim 77, wherein the at least one symptom including expressing, in a measurable amount, at least one or more measurable markers, or a functional equivalent thereto, that has been increased or decreased in the animal model.

80. The animal model as of claim 77, wherein the marker comprises one or more of:
i) one or more of the BAL cytokines, or fragments or functional equivalents thereof, as shown in TABLE 8;
ii) one or more genes, or fragments or functional equivalents thereof, as shown in at least one of TABLES 14, 15 and 16;
iii) one or more genes, or fragments or functional equivalents thereof, encoding for a protein, or fragments or functional equivalents thereof, or antibody that binds to, as shown in at least one of TABLES 19, 20 and 21; and,
iv) one or more genes, or fragments or functional equivalents thereof, encoding for a protein, or fragments or functional equivalents thereof, or antibody that binds thereto, that produces one or more of the biological or chemical processes as shown in at least one of TABLES 17, 18, 22 and 23.

81. A method of producing the animal model of claim 77, comprising: maintaining the animal in a specific chemical-free environment and sensitizing the animal with the at least one chemical found in smoke and the at least one toxin including LPS.

82. A method of producing the animal of claim 81, wherein at least a part of the animal's respiratory system is sensitized.

83. A method of producing the animal model of claim 82, comprising: maintaining the animal in a specific chemical-free environment and sensitizing the animal with the at least one chemical found in smoke and the at least one toxin including LPS by multiple sequential exposures.

84. A method of producing the animal of claim 83, wherein at least a part of the animal's respiratory system is sensitized.

85. A method of screening for an agent for effectiveness against at least one COPD-related disease, comprising:
administering at least one agent to the animal of claim 74,
determining whether the agent reduces or aggravates one or more symptoms of the COPD-related disease;
correlating a reduction in one or more symptoms with effectiveness of the agent against the COPD-related disease; or
correlating a lack of reduction in one or more symptoms with ineffectiveness of the agent.

86. The method of claim 85, including determining macrophage infiltration by measuring expression of one or more markers wherein the marker comprises one or more of:
i) one or more of the BAL cytokines, or fragments or functional equivalents thereof, as shown in TABLE 8;
ii) one or more genes, or fragments or functional equivalents thereof, as shown in at least one of TABLES 14, 15 and 16;
iii) one or more genes, or fragments or functional equivalents thereof, encoding for a protein, or fragments or functional equivalents thereof, as shown in at least one of TABLES 19, 20 and 21; and,
iv) one or more genes, or fragments or functional equivalents thereof, encoding for a protein, or fragments or functional equivalents thereof, that produces one or more of the biological or chemical processes as shown in at least one of TABLES 17, 18, 22 and 23.

87. The method of claim 86, wherein all method steps are performed in vitro.

88. The animal model of claim 15, wherein the chemical and LPS exposure causes at least a differential inflammatory response in the animal without causing acute morbidity.

89. The animal model of claim 15, wherein at least one marker is differentially expressed at a 2-fold change or greater.

90. The animal model of claim 15, wherein at least one marker is differentially expressed at a 10-fold change or greater.

91. The animal model of claim 15, wherein the animal model is a nonhuman vertebrate.

92. The animal model of claim 15, wherein the animal model is a mouse, rat, rabbit, or primate.

93. The method of claim 1, wherein the mouse is an AKR/J strain mouse.

94. The method of claim 1, wherein the test animal and the control animal are littermates.

95. A cell line obtainable from the animal model of claim 15.

96. A cell isolated from an animal model of claim 15.
